(19) United States
(12) Patent Application Publication
(10) Pub. No.: US 2012/0058484 A1
(43) Pub. Date: Mar. 8, 2012

(54) METHODS AND MATERIALS FOR ASSESSING A MAMMAL'S SUSCEPTIBILITY FOR VENOUS THROMBOEMBOLISM

(76) Inventor: John A. Heit, Rochester, MN (US)

(21) Appl. No.: 13/223,993

(22) Filed: Sep. 1, 2011

Related U.S. Application Data
(60) Provisional application No. 61/379,630, filed on Sep. 2, 2010.

(51) Int. Cl. C12Q 1/68; G01N 33/566; H01J 49/26; G01N 33/50

(52) U.S. Cl. 435/6.12; 436/94; 436/501; 250/282

(57) ABSTRACT

This document provides methods and materials involved in assessing a mammal (e.g., a human) for a susceptibility to develop venous thromboembolism. For example, methods and materials for using a genetic variation in an ABO nucleic acid (e.g., rs2519093) to assess an individual's risk for developing venous thromboembolism are provided.

### CROSS-REFERENCE TO RELATED APPLICATIONS

This application claims the benefit of U.S. Provisional Application Ser. No. 61/379,630, filed Sep. 2, 2010. The disclosure of the prior application is considered part of (and is incorporated by reference in) the disclosure of this application.

### STATEMENT AS TO FEDERALLY SPONSORED RESEARCH

Funding for the work described herein was provided by the federal government under grant number HL083141 awarded by the National Heart, Lung, and Blood Institute. The federal government has certain rights in the invention.

### BACKGROUND

1. Technical Field

This document relates to methods and materials involved in assessing a mammal (e.g., a human) for a susceptibility to develop venous thromboembolism. For example, this document provides methods and materials for using a genetic variation in an ABO nucleic acid (e.g., rs2519093) to assess an individual's risk for developing venous thromboembolism.

2. Background Information

Venous thromboembolism can present as a deep vein thrombosis (e.g., a blood clot occluding the lumen of large veins) or a pulmonary embolism (e.g., a dislodged deep vein thrombosis that embolized and obstructed one or more arteries to the lung). Venous thromboembolism is a frequent disease with an annual incidence of about 1 per 1000 in the general population (Heit, Arteriosclerosis, Thrombosis and Vascular Biology, 28(3):370-2 (2008)). Venous thromboembolism is responsible for a substantial public health burden, with an estimated one-week mortality rate resulting from disease of about 4% in patients with deep vein thrombosis and 30% in patients with pulmonary embolism (Heit et al., Arch. Intern. Med., 159:445-53 (1999)).

### SUMMARY

This document provides methods and materials involved in assessing a mammal (e.g., a human) for a susceptibility to develop venous thromboembolism. For example, this document provides methods and materials for using a genetic variation in an ABO nucleic acid (e.g., rs2519093) to assess an individual's risk for developing venous thromboembolism. Having the ability to identify a mammal (e.g., a human) as being likely to develop a venous thromboembolism can allow clinicians to alert patients to their risk and direct those patients to perform certain activities to reduce the likelihood of developing venous thromboembolism. For example, a person identified as having a susceptibility to develop venous thromboembolism based on a genetic analysis provided herein (e.g., the presence of a genetic variation in an ABO nucleic acid such as rs2519093) can be instructed to (a) perform active movements (e.g., move their legs often or take a walk during long plane trips, car trips, and other situations in which they are sitting or lying down for long periods of time), (b) undergo heparin prophylaxis (e.g., low doses of heparin injected under the skin), and/or (c) wear clothes items that provide intermittent pneumatic compression (e.g., soft boots that automatically and gently squeeze the calves periodically).

In general, one aspect of this document features a method for identifying a mammal having increased susceptibility to develop venous thromboembolism. The method comprises, or consists essentially of, (a) determining whether or not a mammal contains a T allele of rs2519093, and (b) classifying the mammal as having an increased susceptibility to develop venous thromboembolism if the mammal contains the T allele. The mammal can be a human. The method can comprise determining whether or not the mammal is heterozygous for the T allele, wherein the mammal has an increased susceptibility to develop venous thromboembolism if the mammal is heterozygous for the T allele. The method can comprise determining whether or not the mammal is homozygous for the T allele, wherein the mammal has an increased susceptibility to develop venous thromboembolism if the mammal is homozygous for the T allele. The method can comprise determining whether or not the mammal contains a G allele of rs6025, a G allele of rs1799963, or a mutant allele of rs8176719.

In another aspect, this document features a method for identifying a mammal having increased susceptibility to develop venous thromboembolism. The method comprises, or consists essentially of, (a) detecting the presence of a mutant T allele of rs2519093 in a mammal, and (b) classifying the mammal as having an increased susceptibility to develop venous thromboembolism based at least in part on the presence of the mutant T allele. The mammal can be a human. The method can comprise determining that the mammal is heterozygous for the mutant T allele. The method can comprise determining that the mammal is homozygous for the mutant T allele. The method can comprise detecting the presence of a G allele of rs6025, a G allele of rs1799963, or a mutant allele of rs8176719.

In another aspect, this document features a method for identifying a mammal having increased susceptibility to develop venous thromboembolism. The method comprises, or consists essentially of, (a) detecting the presence of a mutant T allele of rs2519093 in a mammal, (b) detecting the presence of a G allele of rs6025, a G allele of rs1799963, or a mutant allele of rs8176719, and (c) classifying the mammal as having an increased susceptibility to develop venous thromboembolism based at least in part on the presence of the mutant T allele and the presence of the G allele of rs6025, the G allele of rs1799963, or the mutant allele of rs8176719. The mammal can be a human. The method can comprise determining that the mammal is heterozygous for the mutant T allele. The method can comprise determining that the mammal is homozygous for the mutant T allele.

In another aspect, this document features a method for identifying a mammal having increased susceptibility to develop venous thromboembolism. The method comprises, or consists essentially of, (a) determining whether or not a mammal contains as allele of rs3087505, rs4253399, rs3917862, rs5759224, rs1073897, rs9328375, rs7538157, rs16861990, rs2038024, rs495828, or rs8176719 that is associated with a susceptibility to develop venous thromboembolism, and (b) classifying the mammal as having an increased susceptibility to develop venous thromboembolism if the mammal contains the allele.

In another aspect, this document features a method for identifying a mammal having increased susceptibility to develop venous thromboembolism. The method comprises, or consists essentially of, (a) detecting the presence of, in a mammal, an allele of rs3087505, rs4253399, rs3917862, rs5759224, rs1073897, rs9328375, rs7538157, rs16861990, rs2038024, rs495828, or rs8176719 that is associated with a susceptibility to develop venous thromboembolism, and (b) classifying the mammal as having an increased susceptibility to develop venous thromboembolism based at least in part on the presence of the mutant T allele.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a table listing the demographic and clinical characteristics by case-control status for a study provided herein.

FIG. 2 is a table listing the number of haplotype bins and single nucleotide polymorphisms (SNPs) after Illumina Infinium Custom genotyping SNP selection, design, manufacture, and assay by pathway.

FIG. 3 contains a graph plotting Multidimensional Scaling using ancestry-informative markers by venous thromboembolism case-control status (top), and by race (bottom).

FIG. 4 is a volcano plot showing each candidate gene SNP by each SNP-specific Odds Ratio and −log10(P-value).

FIG. 5 is a Manhattan plot showing each candidate gene SNP by each SNP-specific chromosome location and −log10(P-value).

FIG. 6 is a haploview plot of the ABO gene. The blocks represent the significant associated SNPs. The first three SNPs within haplotype block 1 indicate ABO blood group SNPs (homozygous deletion of rs8176719 determines 0 blood type), while the second, third, fourth and seventh SNP within haplotype block 2 also are associated with VTE but do not determine ABO blood group.

FIG. 7 is a table of the individual and joint population-attributable risks (AR) for significant SNPs assuming a dominant genetic model for each SNP.

FIG. 8 is a table of a comparison between genotyped and serologic blood type.

FIG. 9 is a table of logistic regression results from genotyped and serologic blood type.

FIG. 10 contains multidimensional scaling plots using the 508 ancestry informative markers for cases and controls (A) and by race (B).

FIG. 11. Association results between VTE and the candidate SNPs using Manhattan plot. The x-axis displays the chromosomes, and y-axis displays the −log10 p-values. The significant results (p-value<10E-4) are labeled by gene name and SNP rs number.

FIG. 12. Haploview linkage disequilibrium plot from ABO gene using the candidate SNPs (n=17). Blocks represent SNPs in high linkage disequilibrium. Greater intensity corresponds to a higher level of linkage disequilibrium given by D′. The value inside each cell represents linkage disequilibrium given by r². Larger rectangle on the right outlines the ABO blood group SNPs (homozygous deletion on rs8176719 determines O blood type). Smaller rectangles outline the additional significant SNPs in the ABO gene. Display of the ABO SNPs from HapMap build 36.3 is above the Haploview plot.

FIG. 13 contains Q-Q plots under different models indicated by the plot titles.

FIG. 14 is a STRUCTURE triangle plot of 2962 study individuals and 209 unrelated individuals from HapMap Phase II populations (YRI, CEU, and CHB/JPT) using 494 ancestry informative markers.

FIG. 15A is a Manhattan plot of −log10 (P values) from the case-control association analysis on the merged/imputed VTE data assuming an additive genetic model. The top horizontal line represents the Bonferroni correction. The bottom horizontal line represents SNPs exceeding p=−E-05 in Table 11. FIG. 15B is a plot of the location and linkage disequilibrium for SNPs on chromosome 1q surrounding F5. The top triangle corresponds to F5 6025 (Factor V Leiden) the most significant SNP. FIG. 15C is a plot of the location and linkage disequilibrium for SNPs on chromosome 9q surrounding ABO. The top triangle corresponds to the most significant SNP, rs495828.

FIG. 16A provides the odds ratios and 95% confidence intervals around SNPs significantly associated with VTE for genes within chromosome 1.q. FIG. 16B provides the odds ratios and 95% confidence intervals around SNPs significantly associated with VTE for ABO in chromosome 9.

### DETAILED DESCRIPTION

This document provides methods and materials related to determining whether or not a mammal contains zero, one, or two copies of the T allele of rs2519093. For example, this document provides methods and materials for determining whether or not a mammal is homozygous or heterozygous for the T allele of rs2519093. As described herein, a mammal (e.g., a human) that contains one or two T alleles of rs2519093 can be identified as being likely to develop venous thromboembolism.

The SNP position of rs2519093 is located in the intron 1 region of the ABO blood group nucleic acid (e.g., GenBank® Accession No. NM_{—}020469.2; GI No.: 58331215).

The methods and materials provided herein can be used to determine whether or not nucleic acid of a mammal (e.g., human) contains the T allele of rs2519093. In some cases, the methods and materials provided herein can be used to determine whether both alleles of a mammal contain the T allele of rs2519093, or whether only a single allele of the mammal contains the T allele of rs2519093. The identification of the T allele of rs2519093 can be used to classify or diagnose the mammal as being likely to develop venous thromboembolism. In some cases, a human who has two T alleles at rs2519093 (i.e., a homozygous carrier) can be at higher risk for venous thromboembolism than a human who has one T allele at rs2519093 (i.e., a heterozygous carrier).

Any appropriate method can be used to detect the T allele of rs2519093. For example, mutations can be detected by sequencing cDNA, untranslated sequences, denaturing high performance liquid chromatography (DHPLC; Underhill et al., Genome Res., 7:996-1005 (1997)), allele-specific hybridization (Stoneking et al., Am. J. Hum. Genet., 48:370-382 (1991); and Prince et al., Genome Res., 11(1):152-162 (2001)), allele-specific restriction digests, mutation specific polymerase chain reactions, single-stranded conformational polymorphism detection (Schafer et al., Nat. Biotechnol., 15:33-39 (1998)), infrared matrix-assisted laser desorption/ionization mass spectrometry (WO 99/57318), and combinations of such methods.

In some cases, genomic DNA can be used to detect the T allele of rs2519093. Genomic DNA typically is extracted from a biological sample such as a peripheral blood sample, but can be extracted from other biological samples, including tissues (e.g., mucosal scrapings of the lining of the mouth or from renal or hepatic tissue). Any appropriate method can be used to extract genomic DNA from a blood or tissue sample, including, for example, phenol extraction. In some cases, genomic DNA can be extracted with kits such as the QIAamp® Tissue Kit (Qiagen, Chatsworth, Calif.), the Wizard® Genomic DNA purification kit (Promega, Madison, Wis.), the Puregene DNA Isolation System (Gentra Systems, Minneapolis, Minn.), or the A.S.A.P.3 Genomic DNA isolation kit (Boehringer Mannheim, Indianapolis, Ind.).

An amplification step can be performed before proceeding with the detection method. For example, ABO nucleic acid can be amplified and then directly sequenced. Dye primer sequencing can be used to increase the accuracy of detecting heterozygous samples.

As described herein, the presence of the T allele of rs2519093 in a mammal (e.g., human) can indicate that that mammal has an increased susceptibility of developing venous thromboembolism. In some cases, the presence of the T allele of rs2519093 in a human in combination with the presence of a G allele of rs6025, a G allele of rs1799963, and/or a mutant allele of rs8176719 can indicate that that human has an increased susceptibility of developing venous thromboembolism. In some cases, the presence of the T allele of rs2519093 in a human can indicate that that human has an increased susceptibility of developing venous thromboembolism, especially when that human also includes a family history of inherited thrombophilia and/or venous thromboembolism. For example, the presence of the T allele of rs2519093 in a human in combination with a family history of the Factor V Leiden (G allele of rs6025) and/or prothrombin G20210A (G allele of rs1799963) mutations can indicate that that human has an increased susceptibility of developing venous thromboembolism.

In some cases, any human containing one or two T alleles of rs2519093 can be classified as having an elevated risk of developing venous thromboembolism. For example, a human having one or two T alleles of rs2519093 can be classified as having an elevated risk of developing venous thromboembolism when the human is any age (e.g., less than 65, 60, 55, 50, 45, 40, or 35 years old), does or does not appear to have a family history of venous thromboembolism, or has or has not had a positive or negative diagnostic test for venous thromboembolism. In some cases, a human having one or two T alleles of rs2519093 can be classified as having an elevated risk of developing venous thromboembolism when the human also has a G allele of rs6025, a G allele of rs1799963, and/or a mutant allele of rs8176719.

This document also provides methods and materials related to preventing a human identified as being susceptible to develop venous thromboembolism as described herein from developing venous thromboembolism. For example, a human can be identified as being likely to develop venous thromboembolism if it is determined that the mammal has the T allele of rs2519093. After identifying a human as being likely to develop venous thromboembolism, a health-care professional can take one or more actions that can affect the mammal's care. For example, a health-care professional can record information regarding the presence of the T allele of rs2519093, the presence of one T allele of rs2519093, or the presence of two T alleles of rs2519093 in the human's medical record. In some cases, a health-care professional can record a diagnosis of being likely to develop venous thromboembolism, or otherwise transform the human's medical record, to reflect that the human is susceptible to developing venous thromboembolism. In some cases, a health-care professional can review and evaluate the human's medical record, and can assess multiple preventative treatment strategies for clinical intervention of the human's susceptibility to develop venous thromboembolism. In some cases, a health-care professional can instruct the human to (a) perform active movements (e.g., move their legs often or take a walk during long plane trips, car trips, and other situations in which they are sitting or lying down for long periods of time), (b) undergo heparin prophylaxis (e.g., low doses of heparin injected under the skin), and/or (c) wear clothes items that provide intermittent pneumatic compression (e.g., soft boots that automatically and gently squeeze the calves periodically).

In some cases, one or more of the genetic variations (e.g., SNPs or single nucleotide variants (SNVs)) provided herein (e.g., provided in Table 4 or Table 12) can be used in place of or in combination with rs2519093 to perform a method provided herein. For example, a human containing one or two T alleles of rs495828 can be classified as having an elevated risk of developing venous thromboembolism. For example, a human having one or two T alleles of rs495828 can be classified as having an elevated risk of developing venous thromboembolism when the human is any age (e.g., less than 65, 60, 55, 50, 45, 40, or 35 years old), does or does not appear to have a family history of venous thromboembolism, or has or has not had a positive or negative diagnostic test for venous thromboembolism. In some cases, as described herein, after identifying a human as being likely to develop venous thromboembolism, a health-care professional can take one or more actions that can affect the mammal's care. For example, a health-care professional can record information regarding the presence of the T allele of rs495828, the presence of one T allele of rs495828, or the presence of two T alleles of rs495828 in the human's medical record. In some cases, a health-care professional can record a diagnosis of being likely to develop venous thromboembolism, or otherwise transform the human's medical record, to reflect that the human is susceptible to developing venous thromboembolism. In some cases, a health-care professional can review and evaluate the human's medical record, and can assess multiple preventative treatment strategies for clinical intervention of the human's susceptibility to develop venous thromboembolism. In some cases, a health-care professional can instruct the human to (a) perform active movements (e.g., move their legs often or take a walk during long plane trips, car trips, and other situations in which they are sitting or lying down for long periods of time), (b) undergo heparin prophylaxis (e.g., low doses of heparin injected under the skin), and/or (c) wear clothes items that provide intermittent pneumatic compression (e.g., soft boots that automatically and gently squeeze the calves periodically).

The invention will be further described in the following examples, which do not limit the scope of the invention described in the claims.

### EXAMPLES

### Example 1

### The rs2519093 SNP Can Identify Patients with an Increased Susceptibility to Venous Thromboembolism

A clinic-based case-control study was performed to test genetic variation within genes from the anticoagulant, procoagulant, fibrinolytic, and innate immunity pathways for an association with venous thromboembolism (VTE).

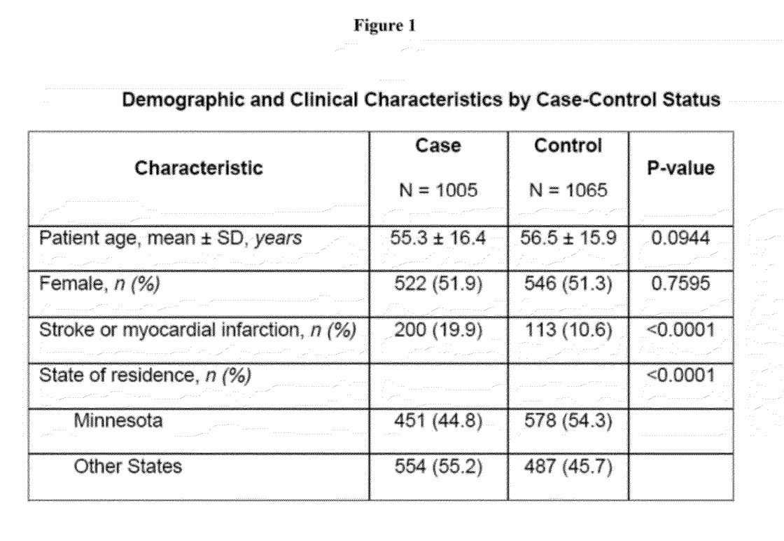

The cases were obtained from Mayo Clinic Caucasian outpatient adults with objectively-diagnosed deep vein thrombosis (DVT) or pulmonary embolism (PE) without active cancer or mechanical thrombosis. Controls were obtained from Mayo Clinic outpatients without VTE, and were frequency-matched on case age group, sex, and race. The primary outcome was VTE status (yes/no) with covariates age at blood draw, sex, stroke, and/or myocardial infarction status, and state of residence (FIG. 1).

To annotate the anticoagulant, procoagulant, fibrinolytic, and innate immunity pathways, 749 candidate genes were selected from three electronic databases: Kyoto Encyclopedia of Genes and Genomes (Complement & Coagulation Cascades); NHLBI Program for Genomic Applications (Fibrinolytic); Univ. of Washington FHCRC Variation Discovery Resource (Innate Immunity).

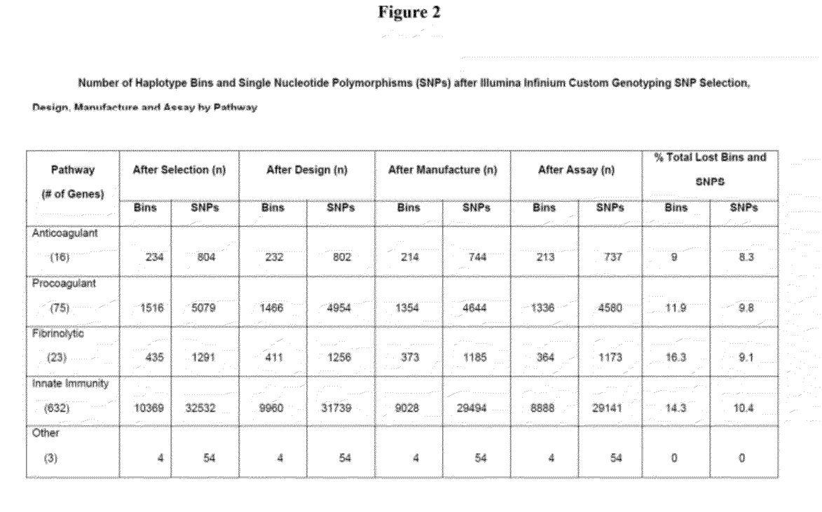

SNPs were selected 10 kb upstream and downstream of each gene using HapMap (http://www.hapmap.org), Perlegen (http://www/perlegen.com), Seattle SNPs (http://pga.mbt.washington.edu/), and NIEHS SNPs (http://egp.gs.washington.edu/) using r² of 0.8 and a minor allele frequency ≧0.005 (FIG. 2).

13,027 SNPs were genotyped using Illumina Infinium iSelect platform, and four SNPs (three ABO blood type and prothrombin G20210A) using TaqMan.

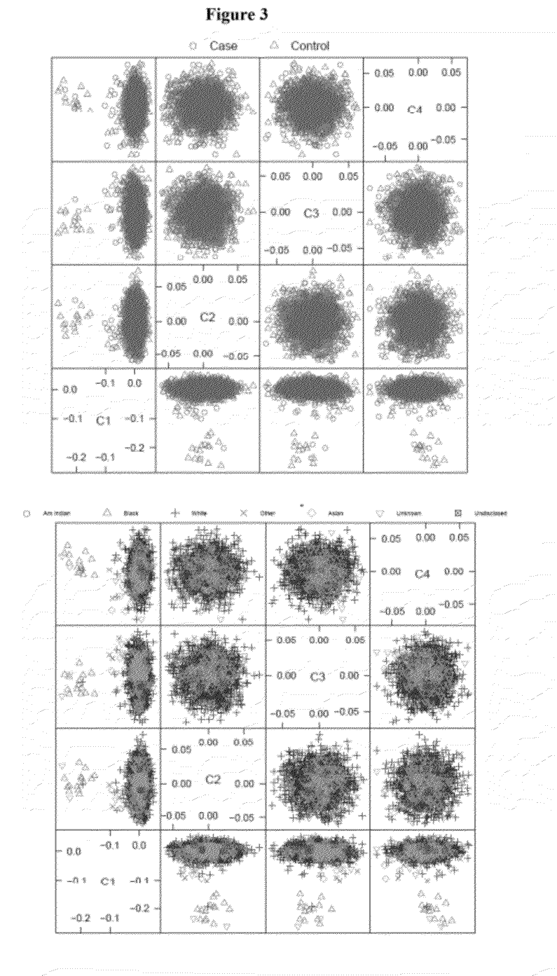

261 individuals were excluded due to inclusion criteria, genotype issues, call rate <0.95, being non-Caucasian, and relatedness. After cleaning, 12,313 SNPs were used. Multidimensional scaling was used for population stratification with 500 ancestry informative markers (FIG. 3).

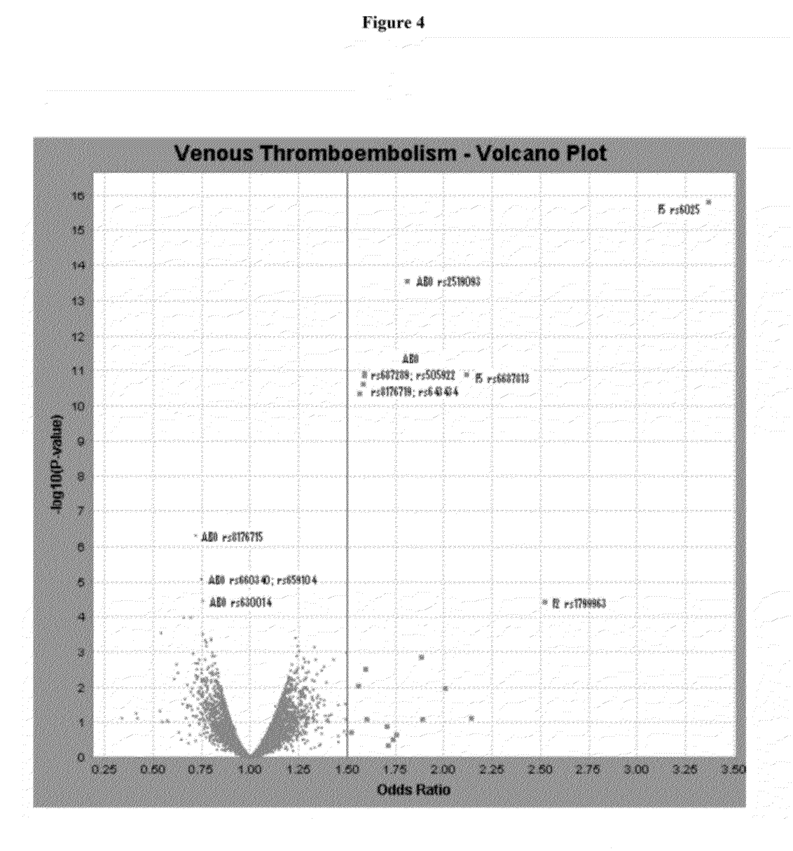

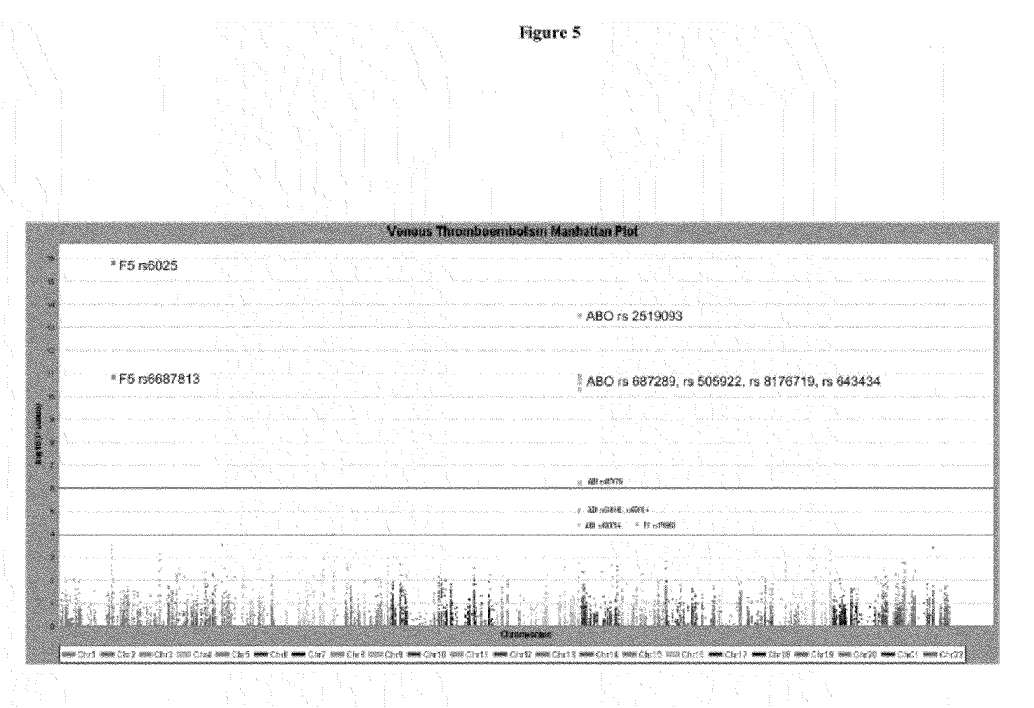

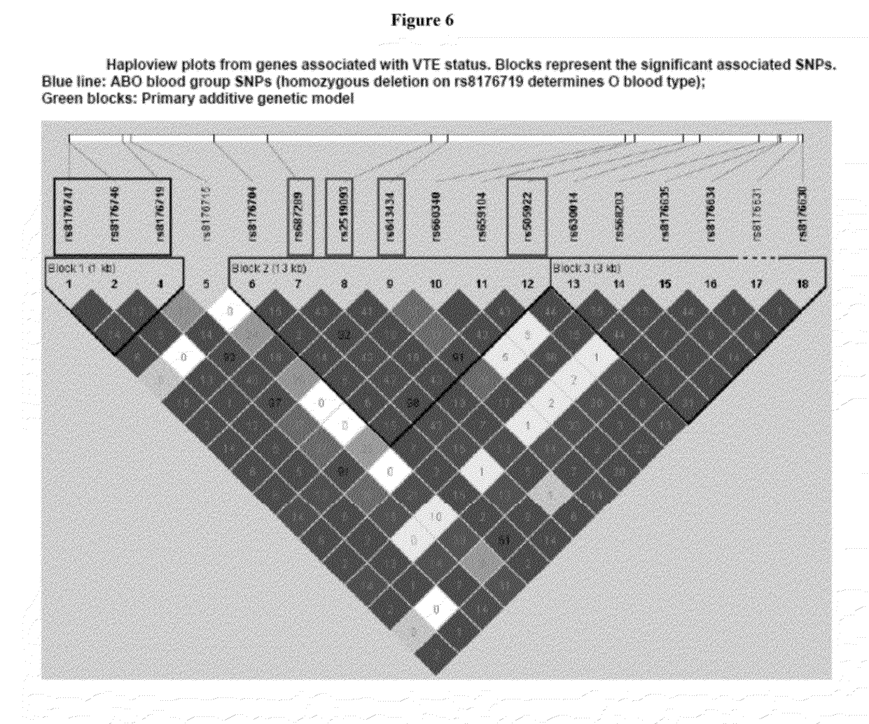

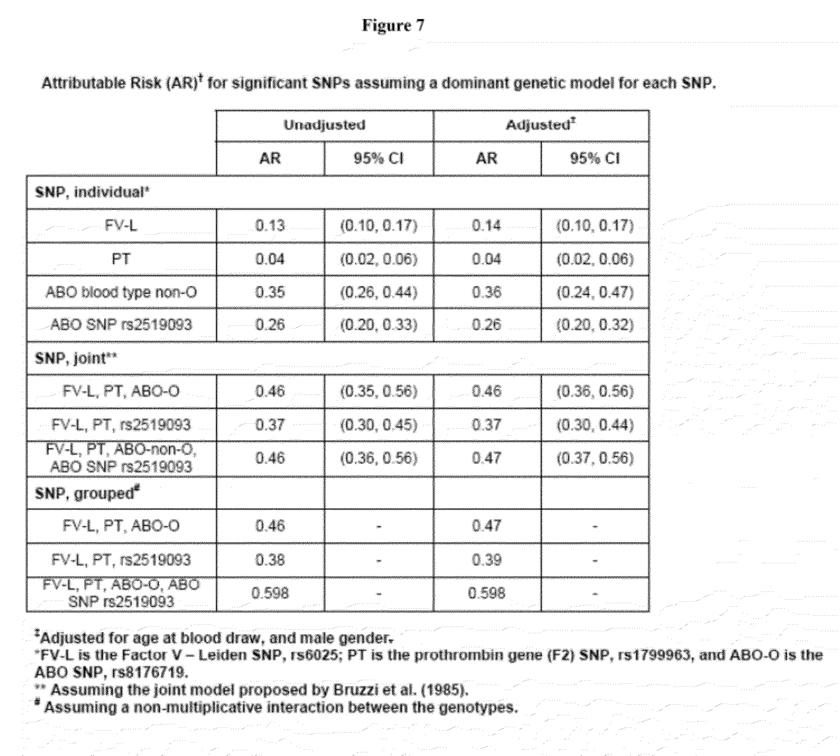

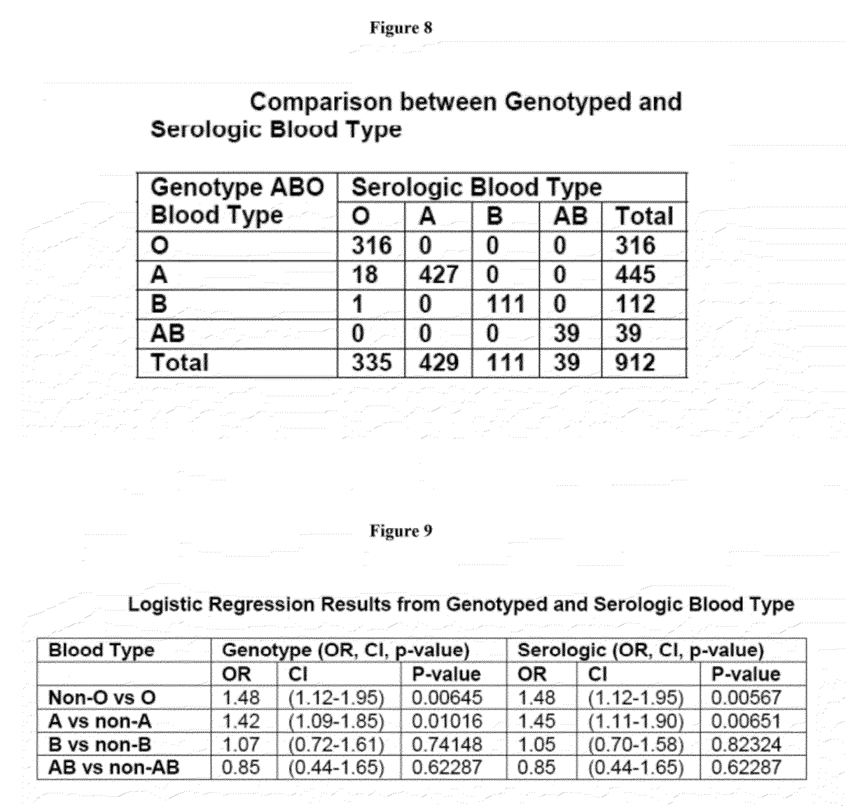

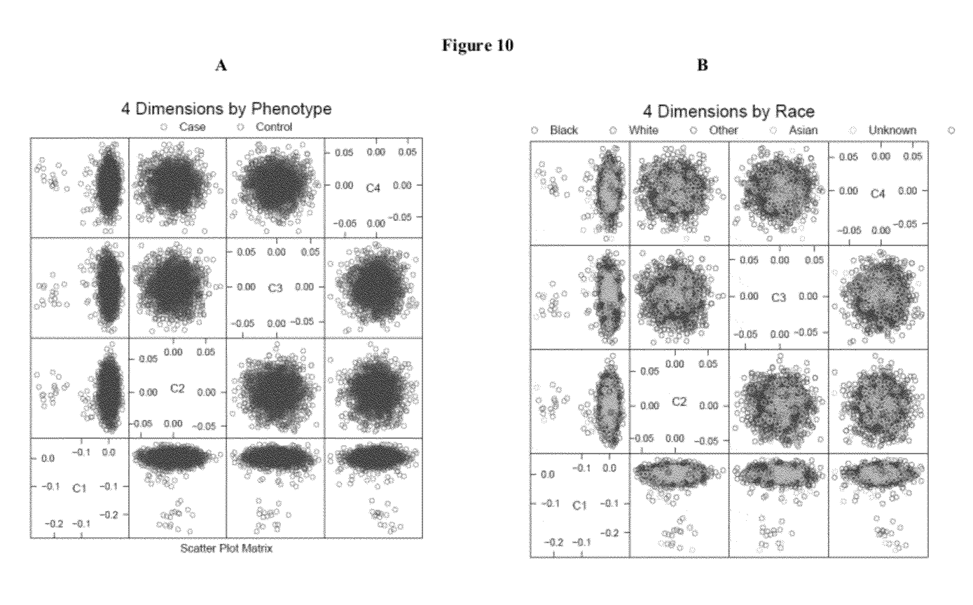

Unconditional logistic regression was performed to test for association between each SNP and VTE status adjusted for age, sex, stroke/MI status, and state of residence. Results using an additive genetic model are shown in FIGS. 4-9. Individual and joint population attributable risks for the risk genotypes for F5 mutation (rs6025), F2 G20210A mutation (rs1799963), the ABO blood type O SNP (rs8176719), and an ABO SNP (rs2519093) are presented in FIG. 7. All analyses were performed using PLINK v 1.05.

These results demonstrate that the presence of the rs2519093 ABO SNP, either alone or in combination with the rs6025 Factor V SNP, the rs1799963 prothrombin SNP, and/or the rs8176719 ABO blood type O SNP, can indicate that a particular person is susceptible to develop VTE. These results also demonstrate that the rs1799963 prothrombin SNP, the rs2519093 ABO SNP, and the rs8176719 ABO blood type O SNP can indicate susceptibility to develop VTE independent of Factor V Leiden status (e.g., rs6025).

### Example 2

### Genetic Variation Within the Anticoagulant, Procoagulant, Fibrinolytic, and Innate Immunity Pathways as Risk Factors for Venous Thromboembolism

The following was performed as part of the work described in Example 1.

### Study Setting and Population

Consecutive Mayo Clinic outpatients age 18 years or older with objectively-diagnosed deep vein thrombosis (DVT) or pulmonary embolism (PE) who resided in the upper Midwest United States and who were referred to the Mayo Clinic Special Coagulation Laboratory or Thrombophilia Center over the study period were approached for study participation. Patients with VTE related to active cancer, an indwelling central venous catheter, transvenous pacemaker or other mechanical cause for thrombosis, a lupus anticoagulant or other antiphospholipid antibodies, vasculitis or a vascular anomaly (e.g., Klippel-Trenaunay), other autoimmune disorders (including heparin-induced thrombocytopenia) or prior bone marrow or liver transplantation were excluded. A DVT or PE was categorized as objectively diagnosed when confirmed by venography, pulmonary angiography, compression venous duplex ultrasonography, ventilation/perfusion lung scan interpreted as high probability for PE, computed tomographic pulmonary angiography, magnetic resonance imaging or pathology examination of thrombus removed at surgery. Clinic-based controls were prospectively selected from persons undergoing outpatient general medical examinations within the Mayo Clinic Divisions of General Internal Medicine and Primary Care Internal Medicine, Department of Internal Medicine, general internal medicine practices that care for patients (>10,000 per year) from the upper Midwest United States. Additional controls were recruited from the Department of Family Medicine and the Mayo Clinic Sports Medicine Center. Controls were frequency matched on the age group (20-29, 30-39, 40-49, 50-59, 60-69, 70-79 years), sex, state of residence and myocardial infarction (MI)/stroke status distribution of the cases, and had no previous diagnosis of VTE or superficial vein thrombosis. Potential controls with active cancer, antiphospholipid antibody syndrome, rheumatologic or other autoimmune disorder, or prior bone marrow or liver transplant were excluded.

For consenting cases and controls, data were collected by in-person questionnaire and medical record review on prior history of VTE and date(s) of VTE (for cases), other thrombotic events and dates of thrombosis (e.g., stroke, MI, peripheral artery thrombosis), current medications and prior exposures (and dates of exposures) that are VTE risk factors, including surgery, hospitalization for acute medical illness, trauma/fracture and neurological disease with leg paresis, and for women, oral contraceptives, obstetric history, and hormone therapy. Cases and controls provided informed consent to use of a venous whole blood sample for leukocyte genomic DNA extraction, storage and use for research addressing the genetics of VTE. The study was approved by the Mayo Clinic Institutional Review Board.

### Candidate Gene Selection

Candidate genes were selected from three electronic databases (i.e., Kyoto Encyclopedia of Genes and Genomes [Complement & Coagulation Cascades]; NHLBI Program for Genomic Applications; Univ. of Washington FHCRC Variation Discovery Resource [Innate Immunity]) that annotate anticoagulant, procoagulant, fibrinolytic, and/or innate immunity pathways. In general, the focus was on platelet, monocyte, neutrophil and endothelial cell agonists, receptors, ligands, signal transduction and adhesion molecules, granule contents and effectors; plasma proteases (procoagulant, anticoagulant, fibrinolytic and complement [including cofactors and receptors]) and inhibitors (e.g., serine protease inhibitors); matrix metalloproteases; inflammatory cytokines and receptors (including leukotrienes and receptors); estrogen, progesterone and androgen receptors, co-regulators and enzymes related to estrogen metabolism; important enzymes for catechol, homocysteine, thromboxane A2 and prostacyclin biosynthesis and metabolism; and 3-Hydroxy-3-Methylglutaryl Coenzyme A (HMG-CoA) reductase.

### SNP Identification and Selection

To identify SNPs for the custom 16,720 bead Illumina Infinium (14,612 SNPs) genotyping panel, genotypes from the genome-wide genotyping projects HapMap (http://www.hapmap.org) and Perlegen (http://www.perlegen.com) were used. Additionally, genotypes from two gene resequencing programs were used: Seattle SNPs (http://pga.mbt.washington.edu/) and NIEHS SNPs (http://egp.gs.washington.edu/). To determine the HapMap and Perlegen SNPs for each of the 764 candidate genes, SNPs 10kb upstream and downstream of each gene were picked. The gene and SNP coordinates were based on NCBI build 35 and dbSNP build 125. If the gene had been resequenced in Seattle SNPs or NIEHS SNPs, genotypes from those sources were used as well. At the time the SNP selection, Seattle SNPs had resequenced 205 of the candidate genes and NIEHS SNPs had resequenced 20.

A hierarchical approach was used for SNP selection. To select Id tagSNPs, IdSelect (Carlson et al., American Journal of Human Genetics., 74:106-20 (2004)) was run on each candidate gene for the Caucasian samples within each public genotype source (HapMap, Perlegen, Seattle, NIEHS). An r² of 0.8 and a minor allele frequency (MAF) cutoff of 0.05 were used with the exception of one gene (GP9-Entrez gene id 2815) where a MAF cutoff of 0.01 was used. To determine the best source of genotypes for each gene where a gene had been resequenced, the source with the higher number of Id bins for the Caucasian samples after removing bins with no tag SNP meeting the minimum Illumina design score (design score=0.4) was used. If each source (e.g., HapMap, Seattle SNPs) had the same number of bins, HapMap was used as the best source because of its higher number of samples (60 unrelated Caucasian samples). HapMap was chosen as the best source for 626 genes, Seattle for 88 genes, Perlegen for 26 genes and NIEHS for 6 genes. Eighteen genes had no tagSNPs because no SNPs had a MAF≧0.05 or met the minimum acceptable Illumina design score or wasn't mapped to the genome reference assembly. If possible, additional tag SNPs were selected when the bin was large. If there were ≧30 or ≧10 SNPs in an Id bin, three and two tag SNPs per bin, respectively, were chose. After completing this process, 12,577 Id tagSNPs, representing 12073 Id bins, were selected for genotyping. 485 bins were dropped due to low design scores.

Nonsynonymous coding (nc)SNPs with a MAF ≧0.005, which met the minimum acceptable Illumina design score, were next selected. For Perlegen and HapMap ncSNPs, the MAF was used for Caucasian samples. For ncSNPs not in those sources, the MAF for Caucasian samples in the Illumina annotation were used. This added 675 ncSNPs to the panel. Next, eight SNPs were added that had been identified in the literature and with collaborators. To test for population stratification, 557 ancestry informative markers were included (Seldin et al., PLoS Genetics., 2:e143 (2006)). Finally, to fill out the panel, 795 additional SNPs were added from the genes and with MAF ≧5%, resulting in a total of 14,612 SNPs. A list of the candidate genes and number of selected SNPs for each gene is provided in Table 1.

TABLE 1

| | | | | | | | Number | Number of | |
|---|---|---|---|---|---|---|---|---|---|
| Gene | | | | | | | of Manufactured | SNPs passed | Number of |
| Name | Gene ID | Description | Chromosome | Gene Location | Gene Start | Gene Stop | SNPs | the Quality Control | Analyzed SNPs |
| A2M | 2 | alpha-2-macroglobulin | 12 | 12p13.31 | 9111571 | 9159825 | 11 | 10 | 10 |
| ABCF1 | 23 | ATP-binding cassette, sub-family F | 6 | 6p21.33 | 30647149 | 30667288 | 3 | 3 | 3 |
| | | (GCN20), member 1 | | | | | | | |
| ABO | 28 | ABO blood group (transferase A, alpha 1- | 9 | 9q34.2 | 135120384 | 135140451 | 20 | 17 | 17 |
| | | 3-N-acetylgalactosaminyltransferase; | | | | | | | |
| | | transferase B, alpha 1-3- | | | | | | | |
| | | galactosyltransferase) | | | | | | | |
| ACE | 1636 | angiotensin I converting enzyme | 17 | 17q23.3 | 58908166 | 58928711 | 7 | 7 | 6 |
| | | (peptidyl-dipeptidase A) 1 | | | | | | | |
| ACE2 | 59272 | angiotensin I converting enzyme | X | Xp22 | 15489077 | 15529058 | 6 | 5 | 5 |
| | | (peptidyl-dipeptidase A) 2 | | | | | | | |
| ACHE | 43 | acetylcholinesterase (Yt blood group) | 7 | 7q22 | 100325551 | 100331477 | 7 | 6 | 6 |
| ACVR1 | 90 | activin A receptor, type I | 2 | 2q23-q24 | 158301207 | 158403036 | 15 | 15 | 15 |
| ACVR2A | 92 | activin A receptor, type IIA | 2 | 2q22.3 | 148319040 | 148404863 | 8 | 8 | 8 |
| ACVR2B | 93 | activin A receptor, type IIB | 3 | 3p22 | 38470794 | 38509637 | 4 | 4 | 4 |
| ADAM17 | 6868 | ADAM metallopeptidase domain 17 | 2 | 2p25 | 9546843 | 9613368 | 9 | 9 | 9 |
| | | (tumor necrosis factor, alpha, converting | | | | | | | |
| | | enzyme) | | | | | | | |
| ADAMTS13 | 11093 | ADAM metallopeptidase with | 9 | 9q34 | 135276941 | 135314328 | 8 | 7 | 7 |
| | | thrombospondin type 1 motif, 13 | | | | | | | |
| ADORA1 | 134 | adenosine A1 receptor | 1 | 1q32.1 | 201363459 | 201403156 | 20 | 19 | 19 |
| ADORA2A | 135 | adenosine A2a receptor | 22 | 22q11.23 | 23153530 | 23168325 | 4 | 3 | 3 |
| ADRB2 | 154 | adrenergic, beta-2-, receptor, surface | 5 | 5q31-q32 | 148186349 | 148188381 | 12 | 11 | 11 |
| AGT | 183 | angiotensinogen (serpin peptidase | 1 | 1q42-q43 | 228904892 | 228916564 | 20 | 20 | 20 |
| | | inhibitor, clade A, member 8) | | | | | | | |
| AGTR1 | 185 | angiotensin II receptor, type 1 | 3 | 3q24 | 149898348 | 149943480 | 17 | 17 | 17 |
| AGTR2 | 186 | angiotensin II receptor, type 2 | X | Xq22-q23 | 115215986 | 115220253 | 5 | 5 | 5 |
| AHSG | 197 | alpha-2-HS-glycoprotein | 3 | 3q27 | 187813544 | 187821801 | 13 | 13 | 13 |
| AIF1 | 199 | allograft inflammatory factor 1 | 6 | 6p21.3 | 31691012 | 31692777 | 3 | 3 | 3 |
| AKT2 | 208 | v-akt murine thymoma viral oncogene | 19 | 19q13.1-q13.2 | 45428064 | 45483105 | 2 | 2 | 2 |
| | | homolog 2 | | | | | | | |
| ALOX12 | 239 | arachidonate 12-lipoxygenase | 17 | 17p13.1 | 6840108 | 6854779 | 14 | 13 | 13 |
| ALOX15 | 246 | arachidonate 15-lipoxygenase | 17 | 17p13.3 | 4480963 | 4491709 | 12 | 11 | 11 |
| ALOX5 | 240 | arachidonate 5-lipoxygenase | 10 | 10q11.2 | 45189635 | 45261571 | 16 | 16 | 16 |
| ALOX5AP | 241 | arachidonate 5-lipoxygenase-activating | 13 | 13q12 | 30207669 | 30236556 | 28 | 26 | 26 |
| | | protein | | | | | | | |
| AMH | 268 | anti-Mullerian hormone | 19 | 19p13.3 | 2200113 | 2203072 | 7 | 6 | 6 |
| AMHR2 | 269 | anti-Mullerian hormone receptor, type II | 12 | 12q13 | 52103908 | 52111579 | 3 | 3 | 3 |
| ANKRD30B | 374860 | ankyrin repeat domain 30B | 18 | 18p11.21 | 14738239 | 14844028 | 8 | 5 | 5 |
| ANXA1 | 301 | annexin A1 | 9 | 9q12-q21.2\| | 74956601 | 74975127 | 15 | 15 | 15 |
| | | | | 9q12-q21.2 | | | | | |
| ANXA2 | 302 | annexin A2 | 15 | 15q22.2 | 58426642 | 58477477 | 52 | 49 | 49 |
| ANXA3 | 306 | annexin A3 | 4 | 4q13-q22 | 79691766 | 79750629 | 13 | 12 | 12 |
| ANXA5 | 308 | annexin A5 | 4 | 4q26-q28\|4q27 | 122808598 | 122837626 | 11 | 11 | 11 |
| APCS | 325 | amyloid P component, serum | 1 | 1q21-q23 | 157824240 | 157825285 | 10 | 10 | 10 |
| APOA2 | 336 | apolipoprotein A-II | 1 | 1q21-q23 | 159458707 | 159460042 | 14 | 14 | 14 |
| APOH | 350 | apolipoprotein H (beta-2-glycoprotein I) | 17 | 17q23-qter | 61638609 | 61656018 | 20 | 19 | 19 |
| APOL2 | 23780 | apolipoprotein L, 2 | 22 | 22q12 | 34952201 | 34965946 | 19 | 19 | 19 |
| APOL3 | 80833 | apolipoprotein L, 3 | 22 | 22q13.1 | 34866323 | 34892171 | 25 | 23 | 23 |
| APP | 351 | amyloid beta (A4) precursor protein | 21 | 21q21.2\|21q21.3 | 26174732 | 26465003 | 52 | 51 | 51 |
| | | (peptidase nexin-II, Alzheimer disease) | | | | | | | |
| AR | 367 | androgen receptor (dihydrotestosterone | X | Xq12 | 66680599 | 66860844 | 6 | 6 | 6 |
| | | receptor; testicular feminization; spinal | | | | | | | |
| | | and bulbar muscular atrophy; Kennedy | | | | | | | |
| | | disease) | | | | | | | |
| ARHGEF1 | 9138 | Rho guanine nucleotide exchange factor | 19 | 19q13.13 | 47079107 | 47103444 | 2 | 2 | 2 |
| | | (GEF) 1 | | | | | | | |
| ARRB1 | 408 | arrestin, beta 1 | 11 | 11q13 | 74654130 | 74740521 | 15 | 15 | 15 |
| ART4 | 420 | ADP-ribosyltransferase 4 (Dombrock | 12 | 12p13-p12 | 14873512 | 14887680 | 14 | 13 | 13 |
| | | blood group) | | | | | | | |
| ASB1 | 51665 | ankyrin repeat and SOCS box-containing 1 | 2 | 2q37 | 239000365 | 239025630 | 6 | 6 | 6 |
| ATF2 | 1386 | activating transcription factor 2 | 2 | 2q32 | 175647252 | 175741143 | 12 | 11 | 11 |
| ATF4 | 468 | activating transcription factor 4 (tax- | 22 | 22q13.1 | 38246515 | 38248637 | 5 | 4 | 4 |
| | | responsive enhancer element B67) | | | | | | | |
| ATRN | 8455 | attractin | 20 | 20p13 | 3399676 | 3579765 | 42 | 42 | 42 |
| AZU1 | 566 | azurocidin 1 (cationic antimicrobial | 19 | 19p13.3 | 778831 | 783017 | 7 | 7 | 7 |
| | | protein 37) | | | | | | | |
| BAG4 | 9530 | BCL2-associated athanogene 4 | 8 | 8p11.23 | 38153263 | 38187694 | 4 | 4 | 4 |
| BATF | 10538 | basic leucine zipper transcription factor, | 14 | 14q24.3 | 75058537 | 75083086 | 10 | 9 | 9 |
| | | ATF-like | | | | | | | |
| BCAM | 4059 | basal cell adhesion molecule (Lutheran | 19 | 19q13.2 | 50004178 | 50016518 | 10 | 10 | 10 |
| | | blood group) | | | | | | | |
| BCL2 | 596 | B-cell CLL/lymphoma 2 | 18 | 18q21.33\|18q21.3 | 58941559 | 59137593 | 62 | 60 | 60 |
| BCL2A1 | 597 | BCL2-related protein A1 | 15 | 15q24.3 | 78040290 | 78050698 | 9 | 9 | 9 |
| BCL3 | 602 | B-cell CLL/lymphoma 3 | 19 | 19q13.1-q13.2 | 49943820 | 49955141 | 4 | 4 | 4 |
| BCL5 | 603 | B-cell CLL/lymphoma 5 | 17 | 17q23.2 | | | 0 | 0 | 0 |
| BCL6 | 604 | B-cell CLL/lymphoma 6 (zinc finger | 3 | 3q27 | 188921859 | 188946169 | 12 | 12 | 12 |
| | | protein 51) | | | | | | | |
| BCO2 | 83875 | beta-carotene oxygenase 2 | 11 | 11q22.3-q23.1 | 111551418 | 111594862 | 12 | 12 | 12 |
| BDKRB1 | 623 | bradykinin receptor B1 | 14 | 14q32.1-q32.2 | 95792312 | 95800853 | 12 | 12 | 12 |
| BDKRB2 | 624 | bradykinin receptor B2 | 14 | 14q32.1-q32.2 | 95740950 | 95780542 | 30 | 29 | 29 |
| BHMT | 635 | betaine-homocysteine methyltransferase | 5 | 5q13.1-q15 | 78443360 | 78463869 | 10 | 10 | 10 |
| BIRC3 | 330 | baculoviral IAP repeat-containing 3 | 11 | 11q22 | 101693404 | 101713675 | 2 | 2 | 2 |
| BLNK | 29760 | B-cell linker | 10 | 10q23.2-q23.33 | 97941445 | 98021316 | 34 | 33 | 33 |
| BMPR1A | 657 | bone morphogenetic protein receptor, | 10 | 10q22.3 | 88506376 | 88674925 | 16 | 15 | 15 |
| | | type IA | | | | | | | |
| BMPR1B | 658 | bone morphogenetic protein receptor, | 4 | 4q22-q24 | 95898151 | 96295099 | 40 | 39 | 39 |
| | | type IB | | | | | | | |
| BMPR2 | 659 | bone morphogenetic protein receptor, | 2 | 2q33-q34 | 202949916 | 203140719 | 12 | 10 | 10 |
| | | type II (serine/threonine kinase) | | | | | | | |
| BPI | 671 | bactericidal/permeability-increasing | 20 | 20q11.23 | 36365966 | 36399319 | 31 | 30 | 30 |
| | | protein | | | | | | | |
| C1QA | 712 | complement component 1, q | 1 | 1p36.12 | 22835705 | 22838762 | 9 | 7 | 7 |
| | | subcomponent, A chain | | | | | | | |
| C1QB | 713 | complement component 1, q | 1 | 1p36.12 | 22852269 | 22860616 | 15 | 14 | 14 |
| | | subcomponent, B chain | | | | | | | |
| C1QBP | 708 | complement component 1, q | 17 | 17p13.3 | 5276823 | 5283195 | 5 | 4 | 4 |
| | | subcomponent binding protein | | | | | | | |
| C1QC | 714 | complement component 1, q | 1 | 1p36.11 | 22842734 | 22847190 | 12 | 10 | 10 |
| | | subcomponent, C chain | | | | | | | |
| C1QL1 | 10882 | complement component 1, q | 17 | 17q21 | 40392587 | 40401170 | 11 | 11 | 11 |
| | | subcomponent-like 1 | | | | | | | |
| C1QL3 | 389941 | complement component 1, q | 10 | 10p13 | 16595748 | 16604010 | 9 | 9 | 9 |
| | | subcomponent-like 3 | | | | | | | |
| C1R | 715 | complement component 1, r | 12 | 12p13 | | | 11 | 11 | 11 |
| | | subcomponent | | | | | | | |
| C1S | 716 | complement component 1, s | 12 | 12p13 | 7038278 | 7048597 | 7 | 6 | 6 |
| | | subcomponent | | | | | | | |
| C2 | 717 | complement component 2 | 6 | 6p21.3 | 32003473 | 32021427 | 18 | 18 | 18 |
| C3 | 718 | complement component 3 | 19 | 19p13.3-p13.2 | 6628846 | 6671662 | 36 | 30 | 30 |
| C3AR1 | 719 | complement component 3a receptor 1 | 12 | 12p13.31 | 8102186 | 8110222 | 4 | 3 | 3 |
| C4A | 720 | complement component 4A (Rodgers | 6 | 6p21.3 | 32090549 | 32111173 | 4 | 1 | 1 |
| | | blood group) | | | | | | | |
| C4BPA | 722 | complement component 4 binding | 1 | 1q32 | 205344230 | 205384940 | 18 | 17 | 17 |
| | | protein, alpha | | | | | | | |
| C4BPB | 725 | complement component 4 binding | 1 | 1q32 | 205328835 | 205339961 | 8 | 8 | 8 |
| | | protein, beta | | | | | | | |
| C5 | 727 | complement component 5 | 9 | 9q33-q34 | 122754434 | 122852375 | 24 | 23 | 23 |
| C5AR1 | 728 | complement component 5a receptor 1 | 19 | 19q13.3-q13.4 | 52504944 | 52517167 | 1 | 0 | 0 |
| C6 | 729 | complement component 6 | 5 | 5p13 | 41178093 | 41249369 | 20 | 19 | 19 |
| C7 | 730 | complement component 7 | 5 | 5p13 | 40945356 | 41018798 | 27 | 25 | 25 |
| C8A | 731 | complement component 8, alpha | 1 | 1p32 | 57093065 | 57156482 | 17 | 16 | 16 |
| | | polypeptide | | | | | | | |
| C9 | 735 | complement component 9 | 5 | 5p14-p12 | 39320758 | 39400412 | 14 | 14 | 14 |
| CADM1 | 23705 | cell adhesion molecule 1 | 11 | 11q23.2 | 114549555 | 114880451 | 59 | 58 | 57 |
| CAMK1D | 57118 | calcium/calmodulin-dependent protein | 10 | 10p13 | 12431589 | 12911741 | 199 | 192 | 191 |
| | | kinase ID | | | | | | | |
| CAPRIN2 | 65981 | caprin family member 2 | 12 | 12p11 | 30753753 | 30798715 | 12 | 12 | 12 |
| CARM1 | 10498 | coactivator-associated arginine | 19 | 19p13.2 | 10843253 | 10894448 | 7 | 7 | 7 |
| | | methyltransferase 1 | | | | | | | |
| CASP1 | 834 | caspase 1, apoptosis-related cysteine | 11 | 11q23 | 104401445 | 104411067 | 9 | 8 | 8 |
| | | peptidase (interleukin 1, beta, convertase) | | | | | | | |
| CASP10 | 843 | caspase 10, apoptosis-related cysteine | 2 | 2q33-q34 | 201755866 | 201802355 | 6 | 6 | 6 |
| | | peptidase | | | | | | | |
| CASP4 | 837 | caspase 4, apoptosis-related cysteine | 11 | 11q22.2-q22.3 | 104318804 | 104344535 | 9 | 9 | 9 |
| | | peptidase | | | | | | | |
| CAV1 | 857 | caveolin 1, caveolae protein, 22 kDa | 7 | 7q31.1 | 115952075 | 115988466 | 14 | 14 | 14 |
| CBS | 875 | cystathionine-beta-synthase | 21 | 21q22.3 | 43346370 | 43369490 | 25 | 22 | 22 |
| CCL1 | 6346 | chemokine (C-C motif) ligand 1 | 17 | 17q12 | 29711512 | 29714365 | 5 | 5 | 5 |
| CCL11 | 6356 | chemokine (C-C motif) ligand 11 | 17 | 17q21.1-q21.2 | 29636800 | 29639312 | 11 | 11 | 11 |
| CCL13 | 6357 | chemokine (C-C motif) ligand 13 | 17 | 17q11.2 | 29707584 | 29709742 | 7 | 6 | 6 |
| CCL15 | 6359 | chemokine (C-C motif) ligand 15 | 17 | 17q11.2 | 31348777 | 31353125 | 10 | 9 | 9 |
| CCL16 | 6360 | chemokine (C-C motif) ligand 16 | 17 | 17q11.2 | 31327648 | 31332636 | 5 | 5 | 5 |
| CCL17 | 6361 | chemokine (C-C motif) ligand 17 | 16 | 16q13 | 55996180 | 56007475 | 8 | 8 | 8 |
| CCL18 | 6362 | chemokine (C-C motif) ligand 18 | 17 | 17q11.2 | 31415756 | 31422954 | 5 | 5 | 5 |
| | | (pulmonary and activation-regulated) | | | | | | | |
| CCL19 | 6363 | chemokine (C-C motif) ligand 19 | 9 | 9p13 | 34679567 | 34681274 | 5 | 5 | 5 |
| CCL2 | 6347 | chemokine (C-C motif) ligand 2 | 17 | 17q11.2-q12 | 29606409 | 29608335 | 7 | 7 | 7 |
| CCL20 | 6364 | chemokine (C-C motif) ligand 20 | 2 | 2q33-q37 | 228386814 | 228390494 | 8 | 7 | 7 |
| CCL21 | 6366 | chemokine (C-C motif) ligand 21 | 9 | 9p13 | 34699002 | 34700147 | 4 | 4 | 4 |
| CCL22 | 6367 | chemokine (C-C motif) ligand 22 | 16 | 16q13 | 55950219 | 55957602 | 9 | 9 | 9 |
| CCL23 | 6368 | chemokine (C-C motif) ligand 23 | 17 | 17q12 | 31364210 | 31369118 | 6 | 5 | 5 |
| CCL24 | 6369 | chemokine (C-C motif) ligand 24 | 7 | 7q11.23 | 75279050 | 75280969 | 11 | 11 | 11 |
| CCL25 | 6370 | chemokine (C-C motif) ligand 25 | 19 | 19p13.2 | 8023934 | 8033547 | 15 | 15 | 15 |
| CCL26 | 10344 | chemokine (C-C motif) ligand 26 | 7 | 7q11.23 | 75236778 | 75257000 | 3 | 2 | 2 |
| CCL3 | 6348 | chemokine (C-C motif) ligand 3 | 17 | 17q12 | 31439715 | 31441619 | 8 | 7 | 7 |
| CCL3L1 | 6349 | chemokine (C-C motif) ligand 3-like 1 | 17 | 17q21.1 | 31647955 | 31649843 | 0 | 0 | 0 |
| CCL4 | 6351 | chemokine (C-C motif) ligand 4 | 17 | 17q12 | 31455333 | 31457127 | 8 | 6 | 6 |
| CCL4L1 | 9560 | chemokine (C-C motif) ligand 4-like 1 | 17 | 17q12 | 31562581 | 31564388 | 0 | 0 | 0 |
| CCL5 | 6352 | chemokine (C-C motif) ligand 5 | 17 | 17q11.2-q12 | 31222608 | 31231490 | 3 | 3 | 3 |
| CCL7 | 6354 | chemokine (C-C motif) ligand 7 | 17 | 17q11.2-q12 | 29621353 | 29623369 | 11 | 10 | 10 |
| CCL8 | 6355 | chemokine (C-C motif) ligand 8 | 17 | 17q11.2 | 29670179 | 29672534 | 13 | 12 | 12 |
| CCR1 | 1230 | chemokine (C-C motif) receptor 1 | 3 | 3p21 | 46218204 | 46224836 | 6 | 6 | 6 |
| CCR10 | 2826 | chemokine (C-C motif) receptor 10 | 17 | 17q21.1-q21.3 | 38084946 | 38087371 | 3 | 3 | 3 |
| CCR2 | 1231 | chemokine (C-C motif) receptor 2 | 3\|NT_113884.1 | | 42313 | 49515 | 2 | 2 | 2 |
| CCR3 | 1232 | chemokine (C-C motif) receptor 3 | 3 | 3p21.3 | 46258692 | 46283166 | 14 | 14 | 14 |
| CCR4 | 1233 | chemokine (C-C motif) receptor 4 | 3 | 3p24 | 32968070 | 32971407 | 3 | 3 | 3 |
| CCR5 | 1234 | chemokine (C-C motif) receptor 5 | 3 | 3p21.31 | 46386637 | 46392701 | 2 | 2 | 2 |
| CCR6 | 1235 | chemokine (C-C motif) receptor 6 | 6 | 6q27 | 167445285 | 167472619 | 20 | 20 | 20 |
| CCR7 | 1236 | chemokine (C-C motif) receptor 7 | 17 | 17q12-q21.2 | 35963547 | 35975250 | 5 | 5 | 5 |
| CCR8 | 1237 | chemokine (C-C motif) receptor 8 | 3 | 3p22 | 39346219 | 39351077 | 4 | 4 | 4 |
| CCR9 | 10803 | chemokine (C-C motif) receptor 9 | 3 | 3p21.3 | 45903023 | 45919671 | 12 | 12 | 11 |
| CCRL1 | 51554 | chemokine (C-C motif) receptor-like 1 | 3 | 3q22 | 133798784 | 133804072 | 2 | 2 | 2 |
| CCRL2 | 9034 | chemokine (C-C motif) receptor-like 2 | 3 | 3p21 | 46423725 | 46426018 | 0 | 0 | 0 |
| CD14 | 929 | CD14 molecule | 5 | 5q22-q32\| | 139991501 | 139993439 | 5 | 4 | 4 |
| | | | | 5q31.1 | | | | | |
| CD180 | 4064 | CD180 molecule | 5 | 5q12 | 66513872 | 66528368 | 15 | 15 | 15 |
| CD1B | 910 | CD1b molecule | 1 | 1q22-q23 | 156564364 | 156567945 | 5 | 5 | 5 |
| CD1C | 911 | CD1c molecule | 1 | 1q22-q23 | 156526187 | 156531188 | 2 | 2 | 2 |
| CD1D | 912 | CD1d molecule | 1 | 1q22-q23 | 156416361 | 156422841 | 9 | 9 | 9 |
| CD22 | 933 | CD22 molecule | 19 | 19q13.1 | 40511919 | 40530104 | 17 | 17 | 17 |
| CD27 | 939 | CD27 molecule | 12 | 12p13 | 6424312 | 6431145 | 23 | 23 | 23 |
| CD276 | 80381 | CD276 molecule | 15 | 15q23-q24 | 71763675 | 71793912 | 8 | 8 | 8 |
| CD28 | 940 | CD28 molecule | 2 | 2q33 | 204279443 | 204310801 | 9 | 8 | 8 |
| CD36 | 948 | CD36 molecule (thrombospondin | 7 | 7q11.2 | 80069459 | 80144262 | 11 | 10 | 10 |
| | | receptor) | | | | | | | |
| CD4 | 920 | CD4 molecule | 12 | 12pter-p12 | 6768912 | 6800237 | 17 | 16 | 16 |
| CD40 | 958 | CD40 molecule, TNF receptor | 20 | 20q12-q13.2 | 44180313 | 44191791 | 12 | 12 | 12 |
| | | superfamily member 5 | | | | | | | |
| CD40LG | 959 | CD40 ligand (TNF superfamily, member | X | Xq26 | 135558002 | 135570215 | 4 | 4 | 4 |
| | | 5, hyper-IgM syndrome) | | | | | | | |
| CD44 | 960 | CD44 molecule (Indian blood group) | 11 | 11p13 | 35116993 | 35210525 | 53 | 52 | 52 |
| CD46 | 4179 | CD46 molecule, complement regulatory | 1 | 1q32 | 205992025 | 206035481 | 45 | 40 | |
| | | protein | | | | | | | |
| CD47 | 961 | CD47 molecule | 3 | 3q13.1-q13.2 | 109244631 | 109292625 | 6 | 6 | 6 |
| CD53 | 963 | CD53 molecule | 1 | 1p13 | 111215344 | 111244081 | 14 | 13 | 13 |
| CD55 | 1604 | CD55 molecule, decay accelerating factor | 1 | 1q32 | 205561488 | 205600470 | 49 | 46 | 46 |
| | | for complement (Cromer blood group) | | | | | | | |
| CD58 | 965 | CD58 molecule | 1 | 1p13 | 116858680 | 116915126 | 7 | 6 | 6 |
| CD59 | 966 | CD59 molecule, complement regulatory | 11 | 11p13 | 33681132 | 33714600 | 49 | 46 | 46 |
| | | protein | | | | | | | |
| CD74 | 972 | CD74 molecule, major histocompatibility | 5 | 5q32 | 149761393 | 149772525 | 12 | 12 | 12 |
| | | complex, class II invariant chain | | | | | | | |
| CD80 | 941 | CD80 molecule | 3 | 3q13.3-q21 | 120725830 | 120761171 | 26 | 26 | 26 |
| CD83 | 9308 | CD83 molecule | 6 | 6p23 | 14225844 | 14245128 | 18 | 18 | 18 |
| CD86 | 942 | CD86 molecule | 3 | 3q21 | 123256911 | 123322673 | 23 | 21 | 21 |
| CD9 | 928 | CD9 molecule | 12 | 12p13.3 | 6179816 | 6217688 | 9 | 9 | 9 |
| CD97 | 976 | CD97 molecule | 19 | 19p13 | 14353213 | 14380535 | 14 | 11 | 11 |
| CDH5 | 1003 | cadherin 5, type 2, VE-cadherin (vascular | 16 | 16q22.1 | 64958064 | 64996190 | 19 | 19 | 19 |
| | | epithelium) | | | | | | | |
| CEBPB | 1051 | CCAAT/enhancer binding protein | 20 | 20q13.1 | 48240783 | 48242619 | 3 | 3 | 3 |
| | | (C/EBP), beta | | | | | | | |
| CEBPD | 1052 | CCAAT/enhancer binding protein | 8 | 8p11.2-p11.1 | 48812029 | 48813279 | 0 | 0 | 0 |
| | | (C/EBP), delta | | | | | | | |
| CFB | 629 | complement factor B | 6 | 6p21.3 | 32021752 | 32027839 | 19 | 19 | 19 |
| CFD | 1675 | complement factor D (adipsin) | 19 | 19p13.3 | 810665 | 814624 | 7 | 7 | 7 |
| CFH | 3075 | complement factor H | 1 | 1q32 | 194887764 | 194983257 | 32 | 14 | 14 |
| CFHR1 | 3078 | complement factor H-related 1 | 1 | 1q32 | 195055484 | 195067942 | 5 | 1 | 1 |
| CFI | 3426 | complement factor I | 4 | 4q25 | 110881297 | 110942783 | 8 | 8 | 8 |
| CFLAR | 8837 | CASP8 and FADD-like apoptosis | 2 | 2q33-q34 | 201689135 | 201737260 | 2 | 2 | 2 |
| | | regulator | | | | | | | |
| CFP | 5199 | complement factor properdin | X | Xp11.4 | 47368569 | 47374648 | 6 | 5 | 5 |
| CHST1 | 8534 | carbohydrate (keratan sulfate Gal-6) | 11 | 11p11.2 | 45627003 | 45643748 | 7 | 7 | 7 |
| | | sulfotransferase 1 | | | | | | | |
| CHUK | 1147 | conserved helix-loop-helix ubiquitous | 10 | 10q24-q25 | 101938114 | 101979334 | 8 | 8 | 8 |
| | | kinase | | | | | | | |
| CISH | 1154 | cytokine inducible SH2-containing | 3 | 3p21.3 | 50618924 | 50624207 | 0 | 0 | 0 |
| | | protein | | | | | | | |
| CITED2 | 10370 | Cbp/p300-interacting transactivator, with | 6 | 6q23.3 | 139735089 | 139737478 | 3 | 3 | 3 |
| | | Glu/Asp-rich carboxy-terminal domain, 2 | | | | | | | |
| CKLF | 51192 | chemokine-like factor | 16 | 16q21 | 65143967 | 65157655 | 5 | 5 | 5 |
| CLC | 1178 | Charcot-Leyden crystal protein | 19 | 19q13.1 | 44913735 | 44920508 | 10 | 9 | 9 |
| CLEC4E | 26253 | C-type lectin domain family 4, member E | 12 | 12p13.31 | 8577168 | 8584825 | 9 | 9 | 9 |
| CNTFR | 1271 | ciliary neurotrophic factor receptor | 9 | 9p13 | 34541431 | 34579722 | 18 | 17 | 17 |
| COL18A1 | 80781 | collagen, type XVIII, alpha 1 | 21 | 21q22.3 | 45649525 | 45758062 | 31 | 28 | 28 |
| COL1A1 | 1277 | collagen, type I, alpha 1 | 17 | 17q21.33 | 45616456 | 45633999 | 11 | 10 | 10 |
| COL1A2 | 1278 | collagen, type I, alpha 2 | 7 | 7q22.1 | 93861809 | 93898480 | 32 | 29 | 29 |
| COL4A1 | 1282 | collagen, type IV, alpha 1 | 13 | 13q34 | 109599311 | 109757497 | 77 | 74 | 74 |
| COLEC10 | 10584 | collectin sub-family member 10 (C-type | 8 | 8q23-q24.1 | 120148627 | 120188388 | 15 | 15 | 15 |
| | | lectin) | | | | | | | |
| COLEC12 | 81035 | collectin sub-family member 12 | 18 | 18pter-p11.3 | 309356 | 490685 | 106 | 103 | 103 |
| CP1 | 50820 | Cleft palate, isolated | 2 | 2q33 | | | 0 | 0 | 0 |
| CPB2 | 1361 | carboxypeptidase B2 (plasma) | 13 | 13q14.11 | 45525323 | 45577212 | 108 | 92 | 92 |
| CR1 | 1378 | complement component (3b/4b) receptor | 1 | 1q32 | 205736096 | 205881733 | 28 | 26 | 26 |
| | | 1 (Knops blood group) | | | | | | | |
| CR2 | 1380 | complement component (3d/Epstein Barr | 1 | 1q32 | 205694293 | 205729863 | 26 | 25 | 25 |
| | | virus) receptor 2 | | | | | | | |
| CRADD | 8738 | CASP2 and RIPK1 domain containing | 12 | 12q21.33-q23.1 | 92595282 | 92768663 | 48 | 48 | 48 |
| | | adaptor with death domain | | | | | | | |
| CREB1 | 1385 | cAMP responsive element binding | 2 | 2q34 | 208102931 | 208171818 | 6 | 6 | 6 |
| | | protein 1 | | | | | | | |
| CREBBP | 1387 | CREB binding protein (Rubinstein-Taybi | 16 | 16p13.3 | 3715056 | 3870122 | 11 | 10 | 10 |
| | | syndrome) | | | | | | | |
| CREM | 1390 | cAMP responsive element modulator | 10 | 10p11.21 | 35455807 | 35541892 | 6 | 6 | 6 |
| CRLF1 | 9244 | cytokine receptor-like factor 1 | 19 | 19p12 | 18565047 | 18578660 | 6 | 5 | 5 |
| CRLF2 | 64109 | cytokine receptor-like factor 2 | X\|Y | Xp22.3; | | | 0 | 0 | 0 |
| | | | | Yp11.3 | | | | | |
| CRP | 1401 | C-reactive protein, pentraxin-related | 1 | 1q21-q23 | 157948703 | 157951003 | 17 | 17 | 17 |
| CSF1R | 1436 | colony stimulating factor 1 receptor, | 5 | 5q32 | 149413051 | 149473128 | 29 | 28 | 28 |
| | | formerly McDonough feline sarcoma | | | | | | | |
| | | viral (v-fms) oncogene homolog | | | | | | | |
| CSF2 | 1437 | colony stimulating factor 2 (granulocyte- | 5 | 5q31.1 | 131437384 | 131439758 | 10 | 9 | 9 |
| | | macrophage) | | | | | | | |
| CSF2RA | 1438 | colony stimulating factor 2 receptor, | X\|Y | Xp22.32 and | | | 0 | 0 | 0 |
| | | alpha, low-affinity (granulocyte- | | Yp11.3 | | | | | |
| | | macrophage) | | | | | | | |
| CSF2RB | 1439 | colony stimulating factor 2 receptor, beta, | 22 | 22q13.1 | 35648168 | 35664764 | 17 | 17 | 17 |
| | | low-affinity (granulocyte-macrophage) | | | | | | | |
| CSF3 | 1440 | colony stimulating factor 3 (granulocyte) | 17 | 17q11.2-q12 | 35425214 | 35427592 | 6 | 6 | 6 |
| CSF3R | 1441 | colony stimulating factor 3 receptor | 1 | 1p35-p34.3 | 36704231 | 36721096 | 12 | 11 | 11 |
| | | (granulocyte) | | | | | | | |
| CX3CL1 | 6376 | chemokine (C—X3—C motif) ligand 1 | 16 | 16q13 | 55963915 | 55976457 | 9 | 9 | 9 |
| CX3CR1 | 1524 | chemokine (C—X3—C motif) receptor 1 | 3 | 3p21\|3p21.3 | 39279989 | 39296531 | 17 | 16 | 16 |
| CXCL1 | 2919 | chemokine (C—X—C motif) ligand 1 | 4 | 4q21 | 74953973 | 74955817 | 7 | 7 | 7 |
| | | (melanoma growth stimulating activity, | | | | | | | |
| | | alpha) | | | | | | | |
| CXCL10 | 3627 | chemokine (C—X—C motif) ligand 10 | 4 | 4q21 | 77161295 | 77163674 | 11 | 9 | 9 |
| CXCL11 | 6373 | chemokine (C—X—C motif) ligand 11 | 4 | 4q21.2 | 77173859 | 77176257 | 7 | 6 | 6 |
| CXCL12 | 6387 | chemokine (C—X—C motif) ligand 12 | 10 | 10q11.1 | 44185611 | 44200548 | 19 | 19 | 19 |
| | | (stromal cell-derived factor 1) | | | | | | | |
| CXCL13 | 10563 | chemokine (C—X—C motif) ligand 13 (B- | 4 | 4q21 | 78651931 | 78752010 | 5 | 5 | 5 |
| | | cell chemoattractant) | | | | | | | |
| CXCL14 | 9547 | chemokine (C—X—C motif) ligand 14 | 5 | 5q31 | 134934274 | 134942868 | 11 | 11 | 11 |
| CXCL16 | 58191 | chemokine (C—X—C motif) ligand 16 | 17 | 17p13 | 4583577 | 4589972 | 12 | 12 | 12 |
| CXCL2 | 2920 | chemokine (C—X—C motif) ligand 2 | 4 | 4q21 | 75181616 | 75183861 | 1 | 1 | 1 |
| CXCL3 | 2921 | chemokine (C—X—C motif) ligand 3 | 4 | 4q21 | 75121170 | 75123354 | 2 | 2 | 2 |
| CXCL5 | 6374 | chemokine (C—X—C motif) ligand 5 | 4 | 4q13.3 | 75080223 | 75083280 | 7 | 4 | 4 |
| CXCL6 | 6372 | chemokine (C—X—C motif) ligand 6 | 4 | 4q21 | 74921277 | 74923341 | 4 | 4 | 4 |
| | | (granulocyte chemotactic protein 2) | | | | | | | |
| CXCL9 | 4283 | chemokine (C—X—C motif) ligand 9 | 4 | 4q21 | 77141647 | 77147665 | 8 | 7 | 7 |
| CXCR3 | 2833 | chemokine (C—X—C motif) receptor 3 | X | Xq13 | 70752491 | 70755092 | 2 | 2 | 2 |
| CXCR4 | 7852 | chemokine (C—X—C motif) receptor 4 | 2 | 2q21 | 136588389 | 136592195 | 6 | 6 | 6 |
| CXCR5 | 643 | chemokine (C—X—C motif) receptor 5 | 11 | 11q23.3 | 118259777 | 118272181 | 10 | 9 | 9 |
| CXCR6 | 10663 | chemokine (C—X—C motif) receptor 6 | 3 | 3p21 | 45959977 | 45964849 | 8 | 6 | 6 |
| CYBB | 1536 | cytochrome b-245, beta polypeptide | X | Xp21.1 | 37524264 | 37557658 | 5 | 5 | 5 |
| | | (chronic granulomatous disease) | | | | | | | |
| CYP4A11 | 1579 | cytochrome P450, family 4, subfamily A, | 1 | 1p33 | 47167433 | 47180004 | 6 | 5 | 5 |
| | | polypeptide 11 | | | | | | | |
| CYP4F2 | 8529 | cytochrome P450, family 4, subfamily F, | 19 | 19pter-p13.11 | 15849834 | 15869884 | 15 | 11 | 11 |
| | | polypeptide 2 | | | | | | | |
| CYP4F3 | 4051 | cytochrome P450, family 4, subfamily F, | 19 | 19p13.2 | 15612707 | 15632570 | 28 | 25 | 25 |
| | | polypeptide 3 | | | | | | | |
| DCN | 1634 | decorin | 12 | 12q21.33 | 90063166 | 90100937 | 6 | 3 | 3 |
| DDX58 | 23586 | DEAD (Asp-Glu-Ala-Asp) box | 9 | 9p12 | 32445300 | 32516322 | 21 | 18 | 18 |
| | | polypeptide 58 | | | | | | | |
| DEFB1 | 1672 | defensin, beta 1 | 8 | 8p23.1 | 6715507 | 6722939 | 27 | 23 | 23 |
| DEFB118 | 117285 | defensin, beta 118 | 20 | 20q11.21 | 29420089 | 29424825 | 7 | 7 | 7 |
| DEFB127 | 140850 | defensin, beta 127 | 20 | 20p13 | 86186 | 87804 | 8 | 7 | 7 |
| DMBT1 | 1755 | deleted in malignant brain tumors 1 | 10 | 10q26.13 | 124310171 | 124393242 | 12 | 12 | 12 |
| DOCK2 | 1794 | dedicator of cytokinesis 2 | 5 | 5q35.1 | 168996871 | 169442959 | 164 | 160 | 160 |
| DOCK3 | 1795 | dedicator of cytokinesis 3 | 3 | 3p21.2 | 50687676 | 51396669 | 14 | 14 | 14 |
| DUSP1 | 1843 | dual specificity phosphatase 1 | 5 | 5q34 | 172127707 | 172130809 | 7 | 6 | 6 |
| DUSP3 | 1845 | dual specificity phosphatase 3 | 17 | 17q21 | 39199015 | 39211872 | 4 | 4 | 4 |
| EBI3 | 10148 | Epstein-Barr virus induced gene 3 | 19 | 19p13.3 | 4180540 | 4188525 | 6 | 6 | 6 |
| EDN1 | 1906 | endothelin 1 | 6 | 6p24.1 | 12398515 | 12405413 | 11 | 10 | 10 |
| EDN2 | 1907 | endothelin 2 | 1 | 1p34 | 41717033 | 41722884 | 9 | 9 | 9 |
| EDNRA | 1909 | endothelin receptor type A | 4 | 4q31.22 | 148621575 | 148685555 | 16 | 14 | 14 |
| EIF2AK2 | 5610 | eukaryotic translation initiation factor 2- | 2 | 2p22-p21 | 37187203 | 37237572 | 10 | 10 | 10 |
| | | alpha kinase 2 | | | | | | | |
| ELK1 | 2002 | ELK1, member of ETS oncogene family | X | Xp11.2 | 47379864 | 47394964 | 4 | 3 | 3 |
| EMD | 2010 | emerin (Emery-Dreifuss muscular | X | Xq28 | 153260981 | 153263075 | 1 | 1 | 1 |
| | | dystrophy) | | | | | | | |
| EPHB6 | 2051 | EPH receptor B6 | 7 | 7q33-q35 | 142262914 | 142278969 | 10 | 8 | 8 |
| EPHX2 | 2053 | epoxide hydrolase 2, cytoplasmic | 8 | 8p21 | 27404562 | 27458403 | 15 | 15 | 15 |
| EPOR | 2057 | erythropoietin receptor | 19 | 19p13.3-p13.2 | 11349475 | 11356019 | 3 | 3 | 3 |
| ERAP1 | 51752 | endoplasmic reticulum aminopeptidase 1 | 5 | 5q15 | 96122270 | 96169648 | 38 | 38 | 38 |
| ESR1 | 2099 | estrogen receptor 1 | 6 | 6q25.1 | 152170379 | 152466099 | 54 | 53 | 53 |
| ESR2 | 2100 | estrogen receptor 2 (ER beta) | 14 | 14q23.2 | 63763504 | 63875021 | 17 | 17 | 17 |
| ESRRA | 2101 | estrogen-related receptor alpha | 11 | 11q13 | 63829620 | 63840786 | 5 | 4 | 4 |
| ESRRB | 2103 | estrogen-related receptor beta | 14 | 14q24.3 | 75907479 | 76036961 | 62 | 60 | 60 |
| ESRRG | 2104 | estrogen-related receptor gamma | 1 | 1q41 | 214743211 | 215329599 | 251 | 242 | 242 |
| F10 | 2159 | coagulation factor X | 13 | 13q34 | 112825114 | 112851844 | 36 | 31 | 31 |
| F11 | 2160 | coagulation factor XI (plasma | 4 | 4q35 | 187424112 | 187447829 | 21 | 21 | 21 |
| | | thromboplastin antecedent) | | | | | | | |
| F11R | 50848 | F11 receptor | 1 | 1q21.2-q21.3 | 159232608 | 159275358 | 13 | 13 | 13 |
| F12 | 2161 | coagulation factor XII (Hageman factor) | 5 | 5q33-qter | 176761745 | 176769183 | 3 | 3 | 3 |
| F13A1 | 2162 | coagulation factor XIII, A1 polypeptide | 6 | 6p25.3-p24.3 | 6089310 | 6265923 | 248 | 235 | 235 |
| F13B | 2165 | coagulation factor XIII, B polypeptide | 1 | 1q31-q32.1 | 195274944 | 195303020 | 10 | 10 | 10 |
| F2 | 2147 | coagulation factor II (thrombin) | 11 | 11p11 | 46697331 | 46717631 | 9 | 9 | 9 |
| F2R | 2149 | coagulation factor II (thrombin) receptor | 5 | 5q13 | 76047542 | 76067054 | 17 | 17 | 17 |
| F2RL1 | 2150 | coagulation factor II (thrombin) receptor- | 5 | 5q13 | 76150610 | 76166896 | 14 | 13 | 13 |
| | | like 1 | | | | | | | |
| F2RL2 | 2151 | coagulation factor II (thrombin) receptor- | 5 | 5q13 | 75947063 | 75954996 | 25 | 25 | 25 |
| | | like 2 | | | | | | | |
| F2RL3 | 9002 | coagulation factor II (thrombin) receptor- | 19 | 19p12 | 16860826 | 16863830 | 6 | 6 | 6 |
| | | like 3 | | | | | | | |
| F3 | 2152 | coagulation factor III (thromboplastin, | 1 | 1p22-p21 | 94767461 | 94779903 | 6 | 5 | 5 |
| | | tissue factor) | | | | | | | |
| F5 | 2153 | coagulation factor V (proaccelerin, labile | 1 | 1q23 | 167747816 | 167822393 | 51 | 42 | 42 |
| | | factor) | | | | | | | |
| F7 | 2155 | coagulation factor VII (serum | 13 | 13q34 | 112808106 | 112822996 | 25 | 20 | 20 |
| | | prothrombin conversion accelerator) | | | | | | | |
| F8 | 2157 | coagulation factor VIII, procoagulant | X | Xq28 | 153717260 | 153904192 | 10 | 9 | 9 |
| | | component (hemophilia A) | | | | | | | |
| F9 | 2158 | coagulation factor IX (plasma | X | Xq27.1-q27.2 | 138440561 | 138473283 | 12 | 10 | 10 |
| | | thromboplastic component, Christmas | | | | | | | |
| | | disease, hemophilia B) | | | | | | | |
| FADD | 8772 | Fas (TNFRSF6)-associated via death | 11 | 11q13.3 | 69726917 | 69731144 | 4 | 4 | 4 |
| | | domain | | | | | | | |
| FAM132A | 388581 | family with sequence similarity 132, | 1 | 1p36.33 | 1167696 | 1171965 | 1 | 1 | 1 |
| | | member A | | | | | | | |
| FAS | 355 | Fas (TNF receptor superfamily, member | 10 | 10q24.1 | 90740268 | 90765522 | 23 | 21 | 21 |
| | | 6) | | | | | | | |
| FASLG | 356 | Fas ligand (TNF superfamily, member 6) | 1 | 1q23 | 170894808 | 170902636 | 11 | 11 | 10 |
| FCER1G | 2207 | Fc fragment of IgE, high affinity I, | 1 | 1q23 | 159451711 | 159455662 | 15 | 15 | 15 |
| | | receptor for; gamma polypeptide | | | | | | | |
| FCN1 | 2219 | ficolin (collagen/fibrinogen domain | 9 | 9q34 | 136941253 | 136949630 | 7 | 7 | 7 |
| | | containing) 1 | | | | | | | |
| FCN2 | 2220 | ficolin (collagen/fibrinogen domain | 9 | 9q34.3 | 136912479 | 136919187 | 20 | 18 | 18 |
| | | containing lectin) 2 (hucolin) | | | | | | | |
| FCN3 | 8547 | ficolin (collagen/fibrinogen domain | 1 | 1p36.11 | 27568188 | 27573902 | 5 | 4 | 4 |
| | | containing) 3 (Hakata antigen) | | | | | | | |
| FES | 2242 | feline sarcoma oncogene | 15 | 15q26.1 | 89228713 | 89240010 | 6 | 5 | 5 |
| FGA | 2243 | fibrinogen alpha chain | 4 | 4q28 | 155723730 | 155731347 | 13 | 12 | 12 |
| FGB | 2244 | fibrinogen beta chain | 4 | 4q28 | 155703596 | 155711688 | 16 | 14 | 14 |
| FGF1 | 2246 | fibroblast growth factor 1 (acidic) | 5 | 5q31 | 141953306 | 142045812 | 36 | 33 | 33 |
| FGG | 2266 | fibrinogen gamma chain | 4 | 4q28 | 155744736 | 155753352 | 14 | 14 | 14 |
| FGL2 | 10875 | fibrinogen-like 2 | 7 | 7q11.23 | 76660624 | 76667086 | 7 | 7 | 7 |
| FIGF | 2277 | c-fos induced growth factor (vascular | Xp22.31 | 15273639 | 15312498 | 10 | 10 | 10 | |
| | | endothelial growth factor D) | | | | | | | |
| FLNA | 2316 | filamin A, alpha (actin binding protein | Xq28 | 153230091 | 153252845 | 0 | 0 | 0 | |
| | | 280) | | | | | | | |
| FLNB | 2317 | filamin B, beta (actin binding protein | 3 | 3p14.3 | 57969167 | 58133018 | 37 | 34 | 34 |
| | | 278) | | | | | | | |
| FLT1 | 2321 | fms-related tyrosine kinase 1 (vascular | 13 | 13q12 | 27774389 | 27967265 | 54 | 53 | 53 |
| | | endothelial growth factor/vascular | | | | | | | |
| | | permeability factor receptor) | | | | | | | |
| FN1 | 2335 | fibronectin 1 | 2 | 2q34 | 215933422 | 216009036 | 28 | 27 | 27 |
| FOS | 2353 | v-fos FBJ murine osteosarcoma viral | 14 | 14q24.3 | 74815284 | 74818666 | 9 | 8 | 7 |
| | | oncogene homolog | | | | | | | |
| FPR1 | 2357 | formyl peptide receptor 1 | 19 | 19q13.4 | 56940838 | 56946962 | 17 | 17 | 17 |
| FUK | 197258 | fucokinase | 16 | 16q22.1 | 69045999 | 69071678 | 10 | 7 | 7 |
| FUT3 | 2525 | fucosyltransferase 3 (galactoside 3(4)-L- | 19 | 19p13.3 | 5793899 | 5802485 | 10 | 7 | 7 |
| | | fucosyltransferase, Lewis blood group) | | | | | | | |
| GAB1 | 2549 | GRB2-associated binding protein 1 | 4 | 4q31.21 | 144477500 | 144610729 | 17 | 16 | 16 |
| GATA3 | 2625 | GATA binding protein 3 | 10 | 10p15 | 8136673 | 8157170 | 11 | 11 | 11 |
| GFRA1 | 2674 | GDNF family receptor alpha 1 | 10 | 10q26.11 | 117812943 | 118022966 | 60 | 59 | 59 |
| GFRA2 | 2675 | GDNF family receptor alpha 2 | 8 | 8p21.3 | | | 23 | 23 | 23 |
| GHR | 2690 | growth hormone receptor | 5 | 5p13-p12 | 42459783 | 42757683 | 17 | 15 | 15 |
| GLMN | 11146 | glomulin, FKBP associated protein | 1 | 1p22.1 | 92484543 | 92537154 | 5 | 5 | 5 |
| GNA12 | 2768 | guanine nucleotide binding protein (G | 7 | 7p22.2 | 2734265 | 2850485 | 20 | 18 | 18 |
| | | protein) alpha 12 | | | | | | | |
| GNAQ | 2776 | guanine nucleotide binding protein (G | 9 | 9q21 | 79525009 | 79836012 | 32 | 32 | 32 |
| | | protein), q polypeptide | | | | | | | |
| GP1BA | 2811 | glycoprotein Ib (platelet), alpha | 17 | 17pter-p12 | 4776372 | 4779067 | 6 | 6 | 6 |
| | | polypeptide | | | | | | | |
| GP1BB | 2812 | glycoprotein Ib (platelet), beta | 22 | 22g11.21-q11.23\| | 18091066 | 18092297 | 5 | 5 | 5 |
| | | polypeptide | | 22q11.21 | | | | | |
| GP5 | 2814 | glycoprotein V (platelet) | 3 | 3q29 | 195596839 | 195601284 | 4 | 4 | 4 |
| GP6 | 51206 | glycoprotein VI (platelet) | 19 | 19q13.4 | 60216885 | 60241444 | 16 | 16 | 16 |
| GP9 | 2815 | glycoprotein IX (platelet) | 3 | 3q21.3 | 130262335 | 130263939 | 2 | 1 | 1 |
| GPI | 2821 | glucose phosphate isomerase | 19 | 19q13.1 | 39547909 | 39583076 | 5 | 5 | 5 |
| GPR44 | 11251 | G protein-coupled receptor 44 | 11 | 11q12-q13.3 | 60374974 | 60380020 | 6 | 6 | 6 |
| GPR68 | 8111 | G protein-coupled receptor 68 | 14 | 14q31 | 90768629 | 90789977 | 11 | 9 | 9 |
| GRB2 | 2885 | growth factor receptor-bound protein 2 | 17 | 17q24-q25 | 70825752 | 70913385 | 10 | 10 | 10 |
| GYPC | 2995 | glycophorin C (Gerbich blood group) | 2 | 2q14-q21 | 127130154 | 127170716 | 32 | 30 | 30 |
| HABP2 | 3026 | hyaluronan binding protein 2 | 10 | 10q25.3 | 115302775 | 115339348 | 50 | 48 | 48 |
| HCK | 3055 | hemopoietic cell kinase | 20 | 20q11-q12 | 30103718 | 30153320 | 11 | 9 | 9 |
| HDAC4 | 9759 | histone deacetylase 4 | 2 | 2q37.3 | 239634801 | 239987580 | 105 | 102 | 101 |
| HDAC5 | 10014 | histone deacetylase 5 | 17 | 17q21 | 39509647 | 39556540 | 8 | 8 | 8 |
| HDAC7 | 51564 | histone deacetylase 7 | 12 | 12q13.1 | 46462772 | 46499924 | 13 | 13 | 13 |
| HDAC9 | 9734 | histone deacetylase 9 | 7 | 7p21.1 | 18501894 | 19003518 | 177 | 170 | 170 |
| HGF | 3082 | hepatocyte growth factor (hepapoietin A; | 7 | 7q21.1 | 81169380 | 81237388 | 18 | 17 | 17 |
| | | scatter factor) | | | | | | | |
| HLA-A | 3105 | major histocompatibility complex, class I, A | 6 | 6p21.3 | 30018310 | 30021633 | 27 | 9 | 9 |
| HLA-B | 3106 | major histocompatibility complex, class I, B | 6 | 6p21.3 | 31429628 | 31432914 | 29 | 20 | 20 |
| HLA-C | 3107 | major histocompatibility complex, class I, C | 6 | 6p21.3 | 31344508 | 31347834 | 47 | 33 | 33 |
| HLA- | 3108 | major histocompatibility complex, class | 6 | 6p21.3 | 33024373 | 33028831 | 14 | 13 | 13 |
| DMA | | II, DM alpha | | | | | | | |
| HLA- | 3109 | major histocompatibility complex, class | 6 | 6p21.3 | 33010393 | 33016795 | 22 | 22 | 22 |
| DMB | | II, DM beta | | | | | | | |
| HLA- | 3111 | major histocompatibility complex, class | 6 | 6p21.3 | 33079937 | 33085367 | 15 | 15 | 15 |
| DOA | | II, DO alpha | | | | | | | |
| HLA- | 3112 | major histocompatibility complex, class | 6 | 6p21.3 | 32888518 | 32892803 | 22 | 22 | 22 |
| DOB | | II, DO beta | | | | | | | |
| HLA- | 3113 | major histocompatibility complex, class | 6 | 6p21.3 | 33140772 | 33149356 | 23 | 17 | 17 |
| DPA1 | | II, DP alpha 1 | | | | | | | |
| HLA- | 3115 | major histocompatibility complex, class | 6 | 6p21.3 | 33151738 | 33162954 | 28 | 23 | 23 |
| DPB1 | | II, DP beta 1 | | | | | | | |
| HLA- | 3118 | major histocompatibility complex, class | 6 | 6p21.3 | 32817141 | 32823199 | 24 | 20 | 20 |
| DQA2 | | II, DQ alpha 2 | | | | | | | |
| HLA- | 3119 | major histocompatibility complex, class | 6 | 6p21.3 | 32735635 | 32742444 | 1 | 1 | 1 |
| DQB1 | | II, DQ beta 1 | | | | | | | |
| HLA- | 3120 | major histocompatibility complex, class | 6 | 6p21 | 32831853 | 32839308 | 27 | 24 | 24 |
| DQB2 | | II, DQ beta 2 | | | | | | | |
| HLA- | 3122 | major histocompatibility complex, class | 6 | 6p21.3 | 32515625 | 32520801 | 15 | 15 | 15 |
| DRA | | II, DR alpha | | | | | | | |
| HLA- | 3125 | major histocompatibility complex, class | 6 | 6p21.3 | | | 0 | 0 | 0 |
| DRB3 | | II, DR beta 3 | | | | | | | |
| HLA- | 3126 | major histocompatibility complex, class | 6 | 6p21.3 | | | 0 | 0 | 0 |
| DRB4 | | II, DR beta 4 | | | | | | | |
| HLA- | 3127 | major histocompatibility complex, class | 6 | 6p21.3 | 32593129 | 32605984 | 1 | 0 | 0 |
| DRB5 | | II, DR beta 5 | | | | | | | |
| HLA-E | 3133 | major histocompatibility complex, class I, E | 6 | 6p21.3 | 30565250 | 30569077 | 8 | 7 | 7 |
| HLA-F | 3134 | major histocompatibility complex, class I, F | 6 | 6p21.3 | 29799096 | 29803052 | 14 | 13 | 13 |
| HLA-G | 3135 | major histocompatibility complex, class I, G | 6 | 6p21.3 | 29903497 | 29906859 | 11 | 11 | 11 |
| HLA-H | 3136 | major histocompatibility complex, class I, | 6 | 6p21.3 | 29963508 | 29966245 | 7 | 3 | 3 |
| | | H (pseudogene) | | | | | | | |
| HMOX1 | 3162 | heme oxygenase (decycling) 1 | 22 | 22q12\|22q13.1 | 34107087 | 34120194 | 10 | 10 | 10 |
| HRG | 3273 | histidine-rich glycoprotein | 3 | 3q27 | 187866492 | 187878717 | 16 | 14 | 14 |
| HRH1 | 3269 | histamine receptor H1 | 3 | 3p25 | 11153779 | 11279939 | 5 | 5 | 5 |
| HSP90B1 | 7184 | heat shock protein 90 kDa beta (Grp94), | 12 | 12q24.2-q24.3 | 102848319 | 102865833 | 24 | 24 | 24 |
| | | member 1 | | | | | | | |
| HSPA14 | 51182 | heat shock 70 kDa protein 14 | 10 | 10p13 | 14920267 | 14953746 | 6 | 6 | 6 |
| HSPG2 | 3339 | heparan sulfate proteoglycan 2 | 1 | 1p36.1-p34 | 22021324 | 22136337 | 20 | 20 | 20 |
| HTR1A | 3350 | 5-hydroxytryptamine (serotonin) receptor | 5 | 5q11.2-q13 | 63292034 | 63293302 | 4 | 4 | 4 |
| | | 1A | | | | | | | |
| HTRA2 | 27429 | HtrA serine peptidase 2 | 2 | 2p12 | 74610040 | 74614191 | 3 | 3 | 3 |
| ICAM1 | 3383 | intercellular adhesion molecule 1 (CD54), | 19 | 19p13.3-p13.2 | 10242779 | 10258291 | 11 | 10 | 10 |
| | | human rhinovirus receptor | | | | | | | |
| ICAM2 | 3384 | intercellular adhesion molecule 2 | 17 | 17q23-q25 | 59433687 | 59451726 | 4 | 3 | 3 |
| ICEBERG | 59082 | ICEBERG caspase-1 inhibitor | 11 | 11q22.3 | 104513879 | 104515663 | 4 | 4 | 4 |
| IER3 | 8870 | immediate early response 3 | 6 | 6p21.3 | 30818955 | 30820306 | 3 | 3 | 3 |
| IFI16 | 3428 | interferon, gamma-inducible protein 16 | 1 | 1q22 | 157246306 | 157291569 | 13 | 12 | 12 |
| IFNA1 | 3439 | interferon, alpha 1 | 9 | 9p22 | 21430440 | 21431315 | 6 | 5 | 5 |
| IFNA2 | 3440 | interferon, alpha 2 | 9 | 9p22 | 21374254 | 21375396 | 5 | 5 | 5 |
| IFNAR1 | 3454 | interferon (alpha, beta and omega) | 21 | 21q22.1\|21q22.11 | 33619084 | 33653999 | 9 | 9 | 9 |
| | | receptor 1 | | | | | | | |
| IFNAR2 | 3455 | interferon (alpha, beta and omega) | 21 | 21q22.1\|21q22.11 | 33524101 | 33558697 | 17 | 14 | 14 |
| | | receptor 2 | | | | | | | |
| IFNB1 | 3456 | interferon, beta 1, fibroblast | 9 | 9p21 | 21067104 | 21067943 | 6 | 6 | 6 |
| IFNG | 3458 | interferon, gamma | 12 | 12q14 | 66834817 | 66839788 | 6 | 6 | 6 |
| IFNGR1 | 3459 | interferon gamma receptor 1 | 6 | 6q23.3 | 137560315 | 137582200 | 11 | 9 | 9 |
| IFNGR2 | 3460 | interferon gamma receptor 2 (interferon | 21 | 21q22.11 | 33697072 | 33731696 | 11 | 9 | 9 |
| | | gamma transducer 1) | | | | | | | |
| IFT57 | 55081 | intraflagellar transport 57 homolog | 3 | 3q13.13 | 109362349 | 109423938 | 12 | 12 | 12 |
| | | (Chlamydomonas) | | | | | | | |
| IGF1 | 3479 | insulin-like growth factor 1 | 12 | 12q23.2 | 101313806 | 101398454 | 17 | 15 | 15 |
| | | (somatomedin C) | | | | | | | |
| IKBKAP | 8518 | inhibitor of kappa light polypeptide gene | 9 | 9q31 | 110669621 | 110736217 | 42 | 42 | 42 |
| | | enhancer in B-cells, kinase complex- | | | | | | | |
| | | associated protein | | | | | | | |
| IKBKB | 3551 | inhibitor of kappa light polypeptide gene | 8 | 8p11.2 | 42247986 | 42309122 | 10 | 8 | 8 |
| | | enhancer in B-cells, kinase beta | | | | | | | |
| IKBKE | 9641 | inhibitor of kappa light polypeptide gene | 1 | 1q32.1 | 204710419 | 204736845 | 25 | 22 | 22 |
| | | enhancer in B-cells, kinase epsilon | | | | | | | |
| IKBKG | 8517 | inhibitor of kappa light polypeptide gene | X | Xq28 | 153423653 | 153446455 | 2 | 2 | 2 |
| | | enhancer in B-cells, kinase gamma | | | | | | | |
| IL10 | 3586 | interleukin 10 | 1 | 1q31-q32 | 205007571 | 205012462 | 11 | 11 | 11 |
| IL10RA | 3587 | interleukin 10 receptor, alpha | 11 | 11q23 | 117362319 | 117377404 | 18 | 16 | 15 |
| IL10RB | 3588 | interleukin 10 receptor, beta | 21 | 21q22.1-q22.2\| | 33560542 | 33591390 | 19 | 18 | 18 |
| | | | | 21q22.11 | | | | | |
| IL11 | 3589 | interleukin 11 | 19 | 19q13.3-q13.4 | 60567569 | 60573626 | 9 | 8 | 8 |
| IL11RA | 3590 | interleukin 11 receptor, alpha | 9 | 9p13 | 34643932 | 34651884 | 5 | 4 | 4 |
| IL12A | 3592 | interleukin 12A (natural killer cell | 3 | 3q25.33-q26 | 161189323 | 161196500 | 12 | 12 | 12 |
| | | stimulatory factor 1, cytotoxic | | | | | | | |
| | | lymphocyte maturation factor 1, p35) | | | | | | | |
| IL12B | 3593 | interleukin 12B (natural killer cell | 5 | 5q31.1-q33.1 | 158674369 | 158690059 | 16 | 16 | 16 |
| | | stimulatory factor 2, cytotoxic | | | | | | | |
| | | lymphocyte maturation factor 2, p40) | | | | | | | |
| IL12RB1 | 3594 | interleukin 12 receptor, beta 1 | 19 | 19p13.1 | 18031371 | 18058697 | 15 | 12 | 12 |
| IL12RB2 | 3595 | interleukin 12 receptor, beta 2 | 1 | 1p31.3-p31.2 | 67545635 | 67635171 | 21 | 20 | 20 |
| IL13 | 3596 | interleukin 13 | 5 | 5q31 | 132021764 | 132024700 | 7 | 6 | 6 |
| IL13RA1 | 3597 | interleukin 13 receptor, alpha 1 | X | Xq24 | 117745587 | 117812524 | 5 | 5 | 5 |
| IL13RA2 | 3598 | interleukin 13 receptor, alpha 2 | X | Xq13.1-q28 | 114144794 | 114158463 | 3 | 2 | 2 |
| IL15RA | 3601 | interleukin 15 receptor, alpha | 10 | 10p15.1 | 6034340 | 6060148 | 32 | 29 | 29 |
| IL16 | 3603 | interleukin 16 (lymphocyte | 15 | 15q26.3 | 79276274 | 79392157 | 27 | 25 | 25 |
| | | chemoattractant factor) | | | | | | | |
| IL17A | 3605 | interleukin 17A | 6 | 6p12 | 52159144 | 52163395 | 13 | 13 | 13 |
| IL17B | 27190 | interleukin 17B | 5 | 5q33.1 | 148734023 | 148739031 | 6 | 5 | 5 |
| IL17C | 27189 | interleukin 17C | 16 | 16q24 | 87232502 | 87234383 | 7 | 7 | 7 |
| IL17D | 53342 | interleukin 17D | 13 | 13q12.11 | 20175482 | 20195237 | 7 | 6 | 6 |
| IL17F | 112744 | interleukin 17F | 6 | 6p12 | 52209443 | 52217257 | 14 | 14 | 14 |
| IL17RB | 55540 | interleukin 17 receptor B | 3 | 3p21.1 | 53855617 | 53874867 | 10 | 9 | 9 |
| IL18 | 3606 | interleukin 18 (interferon-gamma- | 11 | 11q22.2-q22.3 | 111519186 | 111540050 | 6 | 6 | 6 |
| | | inducing factor) | | | | | | | |
| IL18R1 | 8809 | interleukin 18 receptor 1 | 2 | 2q12 | 102345529 | 102381650 | 15 | 14 | 14 |
| IL18RAP | 8807 | interleukin 18 receptor accessory protein | 2 | 2q12 | 102401686 | 102435457 | 15 | 15 | 15 |
| IL19 | 29949 | interleukin 19 | 1 | 1q32.2 | 205038838 | 205082948 | 18 | 18 | 18 |
| IL1A | 3552 | interleukin 1, alpha | 2 | 2q14 | 113247963 | 113259442 | 9 | 9 | 9 |
| IL1B | 3553 | interleukin 1, beta | 2 | 2q14 | 113303808 | 113310827 | 8 | 8 | 8 |
| IL1F10 | 84639 | interleukin 1 family, member 10 (theta) | 2 | 2q13 | 113542018 | 113549898 | 25 | 24 | 24 |
| IL1F5 | 26525 | interleukin 1 family, member 5 (delta) | 2 | 2q14 | 113532686 | 113538792 | 20 | 20 | 20 |
| IL1F6 | 27179 | interleukin 1 family, member 6 (epsilon) | 2 | 2q12-q14.1 | 113479920 | 113482092 | 9 | 6 | 6 |
| IL1F7 | 27178 | interleukin 1 family, member 7 (zeta) | 2 | 2q12-q14.1 | 113387019 | 113392930 | 10 | 10 | 10 |
| IL1F8 | 27177 | interleukin 1 family, member 8 (eta) | 2 | 2q14 | 113496139 | 113526911 | 14 | 11 | 11 |
| IL1F9 | 56300 | interleukin 1 family, member 9 | 2 | 2q12-q21 | 113452077 | 113459698 | 7 | 7 | 7 |
| IL1R1 | 3554 | interleukin 1 receptor, type I | 2 | 2q12 | 102136834 | 102162766 | 25 | 20 | 20 |
| IL1R2 | 7850 | interleukin 1 receptor, type II | 2 | 2q12 | 101974738 | 102011317 | 17 | 15 | 15 |
| IL1RAP | 3556 | interleukin 1 receptor accessory protein | 3 | 3q28 | 191714585 | 191851995 | 59 | 54 | 54 |
| IL1RAPL2 | 26280 | interleukin 1 receptor accessory protein- | X | Xq22 | 103697652 | 104898478 | 39 | 37 | 37 |
| | | like 2 | | | | | | | |
| IL1RL1 | 9173 | interleukin 1 receptor-like 1 | 2 | 2q12 | 102294394 | 102334929 | 21 | 20 | 20 |
| IL1RL2 | 8808 | interleukin 1 receptor-like 2 | 2 | 2q12 | 102169865 | 102222243 | 23 | 22 | 22 |
| IL1RN | 3557 | interleukin 1 receptor antagonist | 2 | 2q14.2 | 113591941 | 113608064 | 16 | 16 | 16 |
| IL2 | 3558 | interleukin 2 | 4 | 4q26-q27 | 123592075 | 123597100 | 4 | 4 | 4 |
| IL20 | 50604 | interleukin 20 | 1 | 1q32 | 205105777 | 205109191 | 5 | 5 | 5 |
| IL20RA | 53832 | interleukin 20 receptor, alpha | 6 | 6q23.3 | 137362801 | 137407991 | 13 | 13 | 13 |
| IL21 | 59067 | interleukin 21 | 4 | 4q26-q27 | 123739272 | 123761662 | 6 | 6 | 6 |
| IL21R | 50615 | interleukin 21 receptor | 16 | 16p11 | 27321224 | 27369616 | 25 | 24 | 24 |
| IL22 | 50616 | interleukin 22 | 12 | 12q15 | 66928292 | 66933548 | 12 | 12 | 12 |
| IL22RA1 | 58985 | interleukin 22 receptor, alpha 1 | 1 | 1p36.11 | 24318848 | 24342198 | 21 | 20 | 20 |
| IL22RA2 | 116379 | interleukin 22 receptor, alpha 2 | 6 | 6q25.1 | 137506650 | 137536478 | 13 | 12 | 12 |
| IL23R | 149233 | interleukin 23 receptor | 1 | 1p31.3 | 67404757 | 67498250 | 30 | 29 | 29 |
| IL24 | 11009 | interleukin 24 | 1 | 1q32 | 205137412 | 205144107 | 2 | 2 | 2 |
| IL25 | 64806 | interleukin 25 | 14 | 14q11.2 | 22911858 | 22915452 | 9 | 9 | 9 |
| IL26 | 55801 | interleukin 26 | 12 | 12q15 | 66881396 | 66905838 | 14 | 11 | 11 |
| IL27 | 246778 | interleukin 27 | 16 | 16p11 | 28418184 | 28425656 | 1 | 1 | 1 |
| IL28RA | 163702 | interleukin 28 receptor, alpha (interferon, | 1 | 1p36.11 | 24353234 | 24386338 | 19 | 18 | 18 |
| | | lambda receptor) | | | | | | | |
| IL2RA | 3559 | interleukin 2 receptor, alpha | 10 | 10p15-p14 | 6093512 | 6144278 | 43 | 41 | 41 |
| IL2RB | 3560 | interleukin 2 receptor, beta | 22 | 22q13\|22q13.1 | 35851824 | 35875908 | 22 | 20 | 20 |
| IL2RG | 3561 | interleukin 2 receptor, gamma (severe | X | Xq13.1 | 70243984 | 70248128 | 5 | 5 | 5 |
| | | combined immunodeficiency) | | | | | | | |
| IL3 | 3562 | interleukin 3 (colony-stimulating factor, | 5 | 5q31.1 | 131424246 | 131426795 | 6 | 6 | 6 |
| | | multiple) | | | | | | | |
| IL31RA | 133396 | interleukin 31 receptor A | 5 | 5q11.2 | 55183091 | 55248738 | 18 | 18 | 18 |
| IL32 | 9235 | interleukin 32 | 16 | 16p13.3 | 3055314 | 3059669 | 8 | 7 | 7 |
| IL3RA | 3563 | interleukin 3 receptor, alpha (low affinity) | X\|Y | Xp22.3 or | | | 12 | 5 | 5 |
| | | | | Yp11.3 | | | | | |
| IL4 | 3565 | interleukin 4 | 5 | 5q31.1 | 132037272 | 132046267 | 8 | 7 | 7 |
| IL4R | 3566 | interleukin 4 receptor | 16 | 16p12.1-p11.2 | 27232752 | 27283600 | 24 | 24 | 24 |
| IL5 | 3567 | interleukin 5 (colony-stimulating factor, | 5 | 5q31.1 | 131905035 | 131907113 | 6 | 5 | 5 |
| | | eosinophil) | | | | | | | |
| IL5RA | 3568 | interleukin 5 receptor, alpha | 3 | 3p26-p24 | 3086421 | 3127031 | 44 | 44 | 44 |
| IL6 | 3569 | interleukin 6 (interferon, beta 2) | 7 | 7p21 | 22733343 | 22738145 | 11 | 11 | 11 |
| IL6ST | 3572 | interleukin 6 signal transducer (gp130, | 5 | 5q11 | 55272451 | 55326520 | 8 | 7 | 7 |
| | | oncostatin M receptor) | | | | | | | |
| IL7 | 3574 | interleukin 7 | 8 | 8q12-q13 | 79807560 | 79880313 | 12 | 12 | 12 |
| IL7R | 3575 | interleukin 7 receptor | 5 | 5p13 | 35892748 | 35912681 | 14 | 13 | 13 |
| IL8 | 3576 | interleukin 8 | 4 | 4q13-q21 | 74825139 | 74828297 | 3 | 2 | 2 |
| IL8RA | 3577 | interleukin 8 receptor, alpha | 2 | 2q35 | 218735813 | 218739961 | 7 | 6 | 6 |
| IL8RB | 3579 | interleukin 8 receptor, beta | 2 | 2q35 | 218698991 | 218710220 | 5 | 4 | 4 |
| IL9 | 3578 | interleukin 9 | 5 | 5q31.1 | 135255834 | 135259415 | 9 | 7 | 7 |
| IL9R | 3581 | interleukin 9 receptor | X\|Y | Xq28 and | | | 5 | 3 | 3 |
| | | | | Yq12 | | | | | |
| IMMT | 10989 | inner membrane protein, mitochondrial | 2 | 2p11.2\|2 | 86224566 | 86276404 | 12 | 12 | 11 |
| | | (mitofilin) | | | | | | | |
| INHA | 3623 | inhibin, alpha | 2 | 2q33-q36 | 220145198 | 220148671 | 6 | 5 | 5 |
| INHBA | 3624 | inhibin, beta A | 7 | 7p15-p13 | 41695126 | 41709231 | 7 | 7 | 7 |
| INHBB | 3625 | inhibin, beta B | 2 | 2cen-q13 | 120820189 | 120825853 | 7 | 7 | 7 |
| INPP5D | 3635 | inositol polyphosphate-5-phosphatase, | 2 | 2q37.1 | | | 23 | 23 | 23 |
| | | 145 kDa | | | | | | | |
| INS | 3630 | insulin | 11 | 11p15.5 | 2137585 | 2139015 | 4 | 4 | 4 |
| IRAK1 | 3654 | interleukin-1 receptor-associated kinase 1 | X | Xq28 | 152929151 | 152938536 | 1 | 1 | 1 |
| IRAK2 | 3656 | interleukin-1 receptor-associated kinase 2 | 3 | 3p25.3 | 10181563 | 10260427 | 23 | 22 | 22 |
| IRAK3 | 11213 | interleukin-1 receptor-associated kinase 3 | 12 | 12q14.3 | 64869284 | 64928652 | 10 | 9 | 9 |
| IRAK4 | 51135 | interleukin-1 receptor-associated kinase 4 | 12 | 12q12 | 42439047 | 42468166 | 7 | 7 | 7 |
| IRF1 | 3659 | interferon regulatory factor 1 | 5 | 5q31.1 | 131846679 | 131854326 | 7 | 7 | 7 |
| IRF3 | 3661 | interferon regulatory factor 3 | 19 | 19q13.3-q13.4 | 54854641 | 54860936 | 2 | 2 | 2 |
| IRF4 | 3662 | interferon regulatory factor 4 | 6 | 6p25-p23 | 336760 | 356193 | 14 | 12 | 12 |
| IRF7 | 3665 | interferon regulatory factor 7 | 11 | 11p15.5 | 602555 | 605999 | 8 | 8 | 8 |
| IRF8 | 3394 | interferon regulatory factor 8 | 16 | 16q24.1 | 84490275 | 84513713 | 32 | 32 | 32 |
| IRF9 | 10379 | interferon regulatory factor 9 | 14 | 14q11.2 | 23700262 | 23705614 | 6 | 5 | 5 |
| IRS2 | 8660 | insulin receptor substrate 2 | 13 | 13q34 | 109204185 | 109236915 | 16 | 16 | 16 |
| ITCH | 83737 | itchy E3 ubiquitin protein ligase homolog | 20 | 20q11.22 | 32414745 | 32562858 | 8 | 6 | 6 |
| | | (mouse) | | | | | | | |
| ITGA1 | 3672 | integrin, alpha 1 | 5 | 5q11.2 | 52119893 | 52285242 | 77 | 75 | 75 |
| ITGA2 | 3673 | integrin, alpha 2 (CD49B, alpha 2 subunit | 5 | 5q23-q31 | 52320913 | 52426366 | 53 | 47 | 47 |
| | | of VLA-2 receptor) | | | | | | | |
| ITGA2B | 3674 | integrin, alpha 2b (platelet glycoprotein | 17 | 17q21.32 | 39805076 | 39822399 | 4 | 4 | 4 |
| | | IIb of IIb/IIIa complex, antigen CD41) | | | | | | | |
| ITGA4 | 3676 | integrin, alpha 4 (antigen CD49D, alpha 4 | 2 | 2q31.3 | 182029864 | 182110719 | 27 | 27 | 27 |
| | | subunit of VLA-4 receptor) | | | | | | | |
| ITGA5 | 3678 | integrin, alpha 5 (fibronectin receptor, | 12 | 12q11-q13 | 53075312 | 53099317 | 3 | 3 | 3 |
| | | alpha polypeptide) | | | | | | | |
| ITGA6 | 3655 | integrin, alpha 6 | 2 | 2q31.1 | 173000560 | 173079427 | 23 | 20 | 20 |
| ITGA8 | 8516 | integrin, alpha 8 | 10 | 10p13 | 15599094 | 15801776 | 41 | 39 | 39 |
| ITGAD | 3681 | integrin, alpha D | 16 | 16p11.2 | 31312134 | 31345327 | 6 | 6 | 6 |
| ITGAL | 3683 | integrin, alpha L (antigen CD11A (p180), | 16 | 16p11.2 | 30391572 | 30442007 | 17 | 17 | 17 |
| | | lymphocyte function-associated antigen | | | | | | | |
| | | 1; alpha polypeptide) | | | | | | | |
| ITGAM | 3684 | integrin, alpha M (complement | 16 | 16p11.2 | 31178789 | 31251714 | 9 | 9 | 9 |
| | | component 3 receptor 3 subunit) | | | | | | | |
| ITGAV | 3685 | integrin, alpha V (vitronectin receptor, | 2 | 2q31-q32 | 187163045 | 187253873 | 20 | 20 | 20 |
| | | alpha polypeptide, antigen CD51) | | | | | | | |
| ITGAX | 3687 | integrin, alpha X (complement | 16 | 16p11.2 | 31274010 | 31301819 | 11 | 11 | 11 |
| | | component 3 receptor 4 subunit) | | | | | | | |
| ITGB1 | 3688 | integrin, beta 1 (fibronectin receptor, beta | 10 | 10p11.2 | 33229326 | 33287204 | 20 | 19 | 19 |
| | | polypeptide, antigen CD29 includes | | | | | | | |
| | | MDF2, MSK12) | | | | | | | |
| ITGB1BP1 | 9270 | integrin beta 1 binding protein 1 | 2 | 2p25.2 | 9463264 | 9481094 | 5 | 5 | 5 |
| ITGB2 | 3689 | integrin, beta 2 (complement component | 21 | 21q22.3 | 45130296 | 45165303 | 26 | 24 | 24 |
| | | 3 receptor 3 and 4 subunit) | | | | | | | |
| ITGB3 | 3690 | integrin, beta 3 (platelet glycoprotein IIIa, | 17 | 17q21.32 | 42686207 | 42745076 | 23 | 21 | 21 |
| | | antigen CD61) | | | | | | | |
| ITGB3BP | 23421 | integrin beta 3 binding protein (beta3- | 1 | 1p31.3 | 63679050 | 63761423 | 13 | 12 | 12 |
| | | endonexin) | | | | | | | |
| ITGB7 | 3695 | integrin, beta 7 | 12 | 12q13.13 | 51871374 | 51887267 | 5 | 4 | 4 |
| ITIH4 | 3700 | inter-alpha (globulin) inhibitor H4 | 3 | 3p21.1 | 52822046 | 52839734 | 9 | 8 | 8 |
| | | (plasma Kallikrein-sensitive | | | | | | | |
| | | glycoprotein) | | | | | | | |
| ITPR1 | 3708 | inositol 1,4,5-triphosphate receptor, type 1 | 3 | 3p26.1 | 4510034 | 4864286 | 178 | 174 | 173 |
| JAK2 | 3717 | Janus kinase 2 (a protein tyrosine kinase) | 9 | 9p24 | 4975245 | 5117995 | 18 | 15 | 15 |
| JAK3 | 3718 | Janus kinase 3 (a protein tyrosine kinase, | 19 | 19p13.1 | 17797961 | 17819800 | 17 | 16 | 16 |
| | | leukocyte) | | | | | | | |
| JUN | 3725 | jun oncogene | 1 | 1p32-p31 | 59019051 | 59022373 | 4 | 4 | 4 |
| KEL | 3792 | Kell blood group, metallo-endopeptidase | 7 | 7q33 | 142348323 | 142369625 | 6 | 6 | 6 |
| KLHDC2 | 23588 | kelch domain containing 2 | 14 | 14q21.3 | 49304537 | 49319606 | 1 | 0 | 0 |
| KLK1 | 3816 | kallikrein 1 | 19 | 19q13.3 | 56014216 | 56018855 | 14 | 13 | 13 |
| KLK3 | 354 | kallikrein-related peptidase 3 | 19 | 19q13.41 | 56049983 | 56055832 | 16 | 14 | 14 |
| KLKB1 | 3818 | kallikrein B, plasma (Fletcher factor) 1 | 4 | 4q35 | 187385666 | 187416619 | 20 | 19 | 19 |
| KNG1 | 3827 | kininogen 1 | 3 | 3q27 | 187917814 | 187944437 | 25 | 24 | 24 |
| L1RE1 | 4029 | LINE1 retrotransposable element 1 | 22 | 22q11.1-q11.2 | | | 0 | 0 | 0 |
| L1RE2 | 4030 | LINE1 retrotransposable element 2 | 1 | 1q | | | 0 | 0 | 0 |
| LBP | 3929 | lipopolysaccharide binding protein | 20 | 20q11.23 | 36408299 | 36439067 | 20 | 19 | 19 |
| LEP | 3952 | leptin | 7 | 7q31.3 | 127668567 | 127684917 | 6 | 6 | 6 |
| LEPR | 3953 | leptin receptor | 1 | 1p31 | 65658906 | 65875410 | 37 | 37 | 37 |
| LIFR | 3977 | leukemia inhibitory factor receptor alpha | 5 | 5p13-p12 | 38510822 | 38631253 | 12 | 12 | 12 |
| LRP1 | 4035 | low density lipoprotein-related protein 1 | 12 | 12q13-q14 | 55808549 | 55893409 | 14 | 13 | 13 |
| | | (alpha-2-macroglobulin receptor) | | | | | | | |
| LTA | 4049 | lymphotoxin alpha (TNF superfamily, | 6 | 6p21.3 | 31648072 | 31650077 | 16 | 13 | 13 |
| | | member 1) | | | | | | | |
| LTA4H | 4048 | leukotriene A4 hydrolase | 12 | 12q22 | 94918742 | 94953496 | 22 | 22 | 22 |
| LTB | 4050 | lymphotoxin beta (TNF superfamily, | 6 | 6p21.3 | 31656314 | 31658181 | 17 | 13 | 13 |
| | | member 3) | | | | | | | |
| LTB4R | 1241 | leukotriene B4 receptor | 14 | 14q11.2-q12 | 23852357 | 23855992 | 6 | 6 | 6 |
| LTBR | 4055 | lymphotoxin beta receptor (TNFR | 12 | 12p13 | 6363618 | 6370993 | 5 | 4 | 4 |
| | | superfamily, member 3) | | | | | | | |
| LTF | 4057 | lactotransferrin | 3 | 3p21.31 | 46452500 | 46481399 | 8 | 8 | 8 |
| LY75 | 4065 | lymphocyte antigen 75 | 2 | 2q24 | 160368112 | 160469508 | 27 | 27 | 27 |
| LY86 | 9450 | lymphocyte antigen 86 | 6 | 6p25.1 | 6533933 | 6600215 | 46 | 44 | 44 |
| LY9 | 4063 | lymphocyte antigen 9 | 1 | 1q23.3 | 159032552 | 159064669 | 8 | 8 | 8 |
| LY96 | 23643 | lymphocyte antigen 96 | 8 | 8q21.11 | 75066144 | 75103859 | 16 | 15 | 15 |
| MADD | 8567 | MAP-kinase activating death domain | 11 | 11p11.2 | 47247535 | 47308158 | 8 | 8 | 8 |
| MAP2K3 | 5606 | mitogen-activated protein kinase kinase 3 | 17 | 17q11.2 | 21128561 | 21159145 | 7 | 5 | 5 |
| MAP2K4 | 6416 | mitogen-activated protein kinase kinase 4 | 17 | 17p11.2 | 11864860 | 11987776 | 19 | 18 | 18 |
| MAP3K1 | 4214 | mitogen-activated protein kinase kinase | 5 | 5q11.2 | 56146657 | 56227736 | 17 | 17 | 17 |
| | | kinase 1 | | | | | | | |
| MAP3K14 | 9020 | mitogen-activated protein kinase kinase | 17 | 17q21 | 40696271 | 40750197 | 8 | 8 | 8 |
| | | kinase 14 | | | | | | | |
| MAP3K3 | 4215 | mitogen-activated protein kinase kinase | 17 | 17q23.3 | 59053533 | 59127402 | 7 | 7 | 7 |
| | | kinase 3 | | | | | | | |
| MAP3K7 | 6885 | mitogen-activated protein kinase kinase | 6 | 6q15 | 91282074 | 91353628 | 12 | 12 | 12 |
| | | kinase 7 | | | | | | | |
| MAP3K7IP1 | 10454 | mitogen-activated protein kinase kinase | 22 | 22q13.1 | 38125705 | 38163078 | 4 | 4 | 4 |
| | | kinase 7 interacting protein 1 | | | | | | | |
| MAP3K8 | 1326 | mitogen-activated protein kinase kinase | 10 | 10p11.23 | 30762872 | 30790767 | 9 | 8 | 8 |
| | | kinase 8 | | | | | | | |
| MAP4K4 | 9448 | mitogen-activated protein kinase kinase | 2 | 2q11.2-q12 | 101680920 | 101877584 | 20 | 20 | 20 |
| | | kinase kinase 4 | | | | | | | |
| MAPK1 | 5594 | mitogen-activated protein kinase 1 | 22 | 22q11.2\|22q11.21 | 20443946 | 20551970 | 8 | 7 | 6 |
| MAPK11 | 5600 | mitogen-activated protein kinase 11 | 22 | 22q13.33 | 49044269 | 49050906 | 9 | 7 | 7 |
| MAPK14 | 1432 | mitogen-activated protein kinase 14 | 6 | 6p21.3-p21.2 | 36103551 | 36186513 | 15 | 13 | 13 |
| MAPK3 | 5595 | mitogen-activated protein kinase 3 | 16 | 16p11.2 | 30032927 | 30042131 | 4 | 4 | 4 |
| MAPK6 | 5597 | mitogen-activated protein kinase 6 | 15 | 15q21 | 50098739 | 50145754 | 7 | 7 | 7 |
| MAPK8 | 5599 | mitogen-activated protein kinase 8 | 10 | 10q11.22 | 49279693 | 49313189 | 10 | 10 | 10 |
| MAPK9 | 5601 | mitogen-activated protein kinase 9 | 5 | 5q35 | 179595390 | 179640216 | 21 | 20 | 20 |
| MAPKAPK2 | 9261 | mitogen-activated protein kinase- | 1 | 1q32 | 204924912 | 204974249 | 13 | 11 | 11 |
| | | activated protein kinase 2 | | | | | | | |
| MASP1 | 5648 | mannan-binding lectin serine peptidase 1 | 3 | 3q27-q28 | 188418632 | 188492446 | 51 | 49 | 49 |
| | | (C4/C2 activating component of Ra- | | | | | | | |
| | | reactive factor) | | | | | | | |
| MASP2 | 10747 | mannan-binding lectin serine peptidase 2 | 1 | 1p36.3-p36.2 | 11009167 | 11029872 | 3 | 3 | 3 |
| MBL2 | 4153 | mannose-binding lectin (protein C) 2, | 10 | 10q11.2 | 54195146 | 54201466 | 20 | 18 | 18 |
| | | soluble (opsonic defect) | | | | | | | |
| MBL3P | 50639 | mannose-binding lectin (protein A) 1, | 10 | 10q22.3 | | | 0 | 0 | 0 |
| | | pseudogenemannose-binding lectin | | | | | | | |
| | | family member 3, pseudogene | | | | | | | |
| MEFV | 4210 | Mediterranean fever | 16 | 16p13.3 | 3232029 | 3246628 | 8 | 8 | 8 |
| MEN1 | 4221 | multiple endocrine neoplasia I | 11 | 11q13 | 64327564 | 64335342 | 4 | 4 | 4 |
| MGLL | 11343 | monoglyceride lipase | 3 | 3q21.3 | 128890599 | 129024741 | 33 | 31 | 31 |
| MIF | 4282 | macrophage migration inhibitory factor | 22 | 22q11.23 | 22566565 | 22567409 | 10 | 10 | 10 |
| | | (glycosylation-inhibiting factor) | | | | | | | |
| MMP1 | 4312 | matrix metallopeptidase 1 (interstitial | 11 | 11q22.3 | 102165861 | 102174104 | 17 | 16 | 16 |
| | | collagenase) | | | | | | | |
| MMP2 | 4313 | matrix metallopeptidase 2 (gelatinase A, | 16 | 16q13-q21 | 54070589 | 54098104 | 17 | 16 | 16 |
| | | 72 kDa gelatinase, 72 kDa type IV | | | | | | | |
| | | collagenase) | | | | | | | |
| MMP25 | 64386 | matrix metallopeptidase 25 | 16 | 16p13.3 | 3036683 | 3050728 | 10 | 9 | 9 |
| MMP3 | 4314 | matrix metallopeptidase 3 (stromelysin 1, | 11 | 11q22.3 | 102211738 | 102219552 | 11 | 10 | 10 |
| | | progelatinase) | | | | | | | |
| MMP9 | 4318 | matrix metallopeptidase 9 (gelatinase B, | 20 | 20q11.2-q13.1 | 44070954 | 44078607 | 10 | 10 | 10 |
| | | 92 kDa gelatinase, 92 kDa type IV | | | | | | | |
| | | collagenase) | | | | | | | |
| MMRN1 | 22915 | multimerin 1 | 4 | 4q22 | 91035075 | 91094803 | 12 | 12 | 12 |
| MPL | 4352 | myeloproliferative leukemia virus | 1 | 1p34 | 43576062 | 43592722 | 4 | 4 | 4 |
| | | oncogene | | | | | | | |
| MTHFR | 4524 | 5,10-methylenetetrahydrofolate reductase | 1 | 1p36.3 | 11768374 | 11788702 | 19 | 17 | 17 |
| | | (NADPH) | | | | | | | |
| MTR | 4548 | 5-methyltetrahydrofolate-homocysteine | 1 | 1q43 | 235025341 | 235130585 | 13 | 11 | 11 |
| | | methyltransferase | | | | | | | |
| MYD88 | 4615 | myeloid differentiation primary response | 3 | 3p22 | 38155009 | 38159517 | 5 | 5 | 5 |
| | | gene (88) | | | | | | | |
| NAIP | 4671 | NLR family, apoptosis inhibitory protein | 5 | 5q13.1 | 70300066 | 70356697 | 0 | 0 | 0 |
| NCAM1 | 4684 | neural cell adhesion molecule 1 | 11 | 11q23.1 | 112337205 | 112654368 | 75 | 74 | 74 |
| NCOA4 | 8031 | nuclear receptor coactivator 4 | 10 | 10q11.2 | 51242373 | 51260738 | 7 | 7 | 7 |
| NCOR2 | 9612 | nuclear receptor co-repressor 2 | 12 | 12q24 | 123374914 | 123586102 | 51 | 50 | 50 |
| NCR3 | 259197 | natural cytotoxicity triggering receptor 3 | 6 | 6p21.3 | 31664651 | 31668741 | 6 | 5 | 5 |
| NEBL | 10529 | nebulette | 10 | 10p12 | 21110093 | 21503122 | 79 | 73 | 73 |
| NFAM1 | 150372 | NFAT activating protein with ITAM | 22 | 22q13.2 | 41106357 | 41158345 | 19 | 18 | 18 |
| | | motif 1 | | | | | | | |
| NFATC3 | 4775 | nuclear factor of activated T-cells, | 16 | 16q22.2 | 66676876 | 66818338 | 5 | 5 | 5 |
| | | cytoplasmic, calcineurin-dependent 3 | | | | | | | |
| NFATC4 | 4776 | nuclear factor of activated T-cells, | 14 | 14q11.2 | 23907094 | 23918650 | 9 | 9 | 9 |
| | | cytoplasmic, calcineurin-dependent 4 | | | | | | | |
| NFE2 | 4778 | nuclear factor (erythroid-derived 2), | 12 | 12q13 | 52972162 | 52975811 | 3 | 3 | 3 |
| | | 45 kDa | | | | | | | |
| NFE2L1 | 4779 | nuclear factor (erythroid-derived 2)-like 1 | 17 | 17q21.3 | 43480745 | 43493841 | 8 | 7 | 7 |
| NFIL3 | 4783 | nuclear factor, interleukin 3 regulated | 9 | 9q22 | 93211148 | 93225965 | 7 | 7 | 7 |
| NFKB1 | 4790 | nuclear factor of kappa light polypeptide | 4 | 4q24 | 103641518 | 103757507 | 18 | 16 | 16 |
| | | gene enhancer in B-cells 1 (p105) | | | | | | | |
| NFKB2 | 4791 | nuclear factor of kappa light polypeptide | 10 | 10q24 | 104144219 | 104152271 | 4 | 4 | 4 |
| | | gene enhancer in B-cells 2 (p49/p100) | | | | | | | |
| NFKBIA | 4792 | nuclear factor of kappa light polypeptide | 14 | 14q13 | 34940468 | 34943695 | 12 | 11 | 10 |
| | | gene enhancer in B-cells inhibitor, alpha | | | | | | | |
| NFKBIB | 4793 | nuclear factor of kappa light polypeptide | 19 | 19q13.1 | 44082455 | 44091374 | 9 | 8 | 8 |
| | | gene enhancer in B-cells inhibitor, beta | | | | | | | |
| NFKBIE | 4794 | nuclear factor of kappa light polypeptide | 6 | 6p21.1 | 44333881 | 44341503 | 6 | 5 | 5 |
| | | gene enhancer in B-cells inhibitor, | | | | | | | |
| | | epsilon | | | | | | | |
| NFKBIL1 | 4795 | nuclear factor of kappa light polypeptide | 6 | 6p21.3 | 31623351 | 31634585 | 13 | 12 | 12 |
| | | gene enhancer in B-cells inhibitor-like 1 | | | | | | | |
| NFRKB | 4798 | nuclear factor related to kappaB binding | 11 | 11q24-q25 | 129239568 | 129268114 | 11 | 10 | 10 |
| | | protein | | | | | | | |
| NFX1 | 4799 | nuclear transcription factor, X-box | 9 | 9p13.3 | 33280510 | 33361155 | 9 | 9 | 9 |
| | | binding 1 | | | | | | | |
| NLRP12 | 91662 | NLR family, pyrin domain containing 12 | 19 | 19q13.42 | 58988650 | 59019404 | 22 | 21 | 21 |
| NLRP3 | 114548 | NLR family, pyrin domain containing 3 | 1 | 1q44 | 245646098 | 245679033 | 32 | 30 | 30 |
| NMI | 9111 | N-myc (and STAT) interactor | 2 | 2q23 | 151835231 | 151854620 | 8 | 8 | 8 |
| NOS2A | 4843 | nitric oxide synthase 2A (inducible, | 17 | 17q11.2-q12 | 23107919 | 23151682 | 31 | 26 | 26 |
| | | hepatocytes) | | | | | | | |
| NOS3 | 4846 | nitric oxide synthase 3 (endothelial cell) | 7 | 7q36 | 150319080 | 150342609 | 15 | 13 | 13 |
| NOX5 | 79400 | NADPH oxidase, EF-hand calcium | 15 | 15q23 | 67009918 | 67136127 | 12 | 11 | 11 |
| | | binding domain 5 | | | | | | | |
| NPPB | 4879 | natriuretic peptide precursor B | 1 | 1p36.2 | 11840108 | 11841579 | 9 | 9 | 9 |
| NPR1 | 4881 | natriuretic peptide receptor A/guanylate | 1 | 1q21-q22 | 151917737 | 151933088 | 3 | 3 | 3 |
| | | cyclase A (atrionatriuretic peptide | | | | | | | |
| | | receptor A) | | | | | | | |
| NR3C1 | 2908 | nuclear receptor subfamily 3, group C, | 5 | 5q31.3 | 142637689 | 142795270 | 14 | 13 | 13 |
| | | member 1 (glucocorticoid receptor) | | | | | | | |
| NR4A2 | 4929 | nuclear receptor subfamily 4, group A, | 2 | 2q22-q23 | 156889195 | 156897446 | 5 | 5 | 5 |
| | | member 2 | | | | | | | |
| OCLN | 4950 | occludin | 5 | 5q13.1 | 68823875 | 68885889 | 0 | 0 | 0 |
| OLR1 | 4973 | oxidized low density lipoprotein (lectin- | 12 | 12p13.2-p12.3 | 10202166 | 10216057 | 12 | 10 | 10 |
| | | like) receptor 1 | | | | | | | |
| OSMR | 9180 | oncostatin M receptor | 5 | 5p13.1 | 38881893 | 38970159 | 19 | 18 | 18 |
| P2RX1 | 5023 | purinergic receptor P2X, ligand-gated ion | 17 | 17p13.3 | 3746634 | 3766709 | 20 | 17 | 17 |
| | | channel, 1 | | | | | | | |
| P2RY1 | 5028 | purinergic receptor P2Y, G-protein | 3 | 3q25.2 | 154035426 | 154038535 | 2 | 2 | 1 |
| | | coupled, 1 | | | | | | | |
| P4HB | 5034 | procollagen-proline, 2-oxoglutarate 4- | 17 | 17q25 | 77394323 | 77411833 | 2 | 2 | 2 |
| | | dioxygenase (proline 4-hydroxylase), beta | | | | | | | |
| | | polypeptide | | | | | | | |
| PAFAH2 | 5051 | platelet-activating factor acetylhydrolase | 1 | 1p36 | 26158845 | 26197235 | 5 | 5 | 5 |
| | | 2, 40 kDa | | | | | | | |
| PARP4 | 143 | poly (ADP-ribose) polymerase family, | 13 | 13q11 | 23893069 | 23984948 | 33 | 26 | 26 |
| | | member 4 | | | | | | | |
| PAWR | 5074 | PRKC, apoptosis, WT1, regulator | 12 | 12q21 | 78509876 | 78608921 | 4 | 4 | 4 |
| PAX5 | 5079 | paired box 5 | 9 | 9p13 | 36828531 | 37024476 | 83 | 78 | 78 |
| PCSK9 | 255738 | proprotein convertase subtilisin/kexin | 1 | 1p32.3 | 55277808 | 55303111 | 32 | 31 | 31 |
| | | type 9 | | | | | | | |
| PDAP1 | 11333 | PDGFA associated protein 1 | 7 | 7q22.1 | 98830525 | 98844228 | 4 | 4 | 4 |
| PECAM1 | 5175 | platelet/endothelial cell adhesion | 17 | 17q23 | 59753595 | 59817743 | 11 | 10 | 10 |
| | | molecule (CD31 antigen) | | | | | | | |
| PELI1 | 57162 | pellino homolog 1 (Drosophila) | 2 | 2p13.3 | 64173499 | 64192987 | 11 | 10 | 10 |
| PELO | 53918 | pelota homolog (Drosophila) | 5 | 5q11.2 | 52119531 | 52134208 | 10 | 10 | 10 |
| PF4 | 5196 | platelet factor 4 (chemokine (C—X—C | 4 | 4q12-q21 | 75065660 | 75066541 | 8 | 7 | 7 |
| | | motif) ligand 4) | | | | | | | |
| PGLYRP1 | 8993 | peptidoglycan recognition protein 1 | 19 | 19q13.2-q13.3 | 51214281 | 51218163 | 6 | 6 | 6 |
| PGLYRP2 | 114770 | peptidoglycan recognition protein 2 | 19 | 19p13.12 | 15440456 | 15451315 | 7 | 5 | 5 |
| PGLYRP3 | 114771 | peptidoglycan recognition protein 3 | 1 | 1q21 | 151536962 | 151549818 | 5 | 5 | 5 |
| PGR | 5241 | progesterone receptor | 11 | 11q22-q23 | 100414313 | 100506465 | 21 | 20 | 20 |
| PIGR | 5284 | polymeric immunoglobulin receptor | 1 | 1q31-q41 | 205168495 | 205186430 | 6 | 6 | 6 |
| PLA2G2A | 5320 | phospholipase A2, group IIA (platelets, | 1 | 1p35 | 20174518 | 20179496 | 14 | 13 | 13 |
| | | synovial fluid) | | | | | | | |
| PLA2G2D | 26279 | phospholipase A2, group IID | 1 | 1p36.12 | 20311019 | 20318595 | 19 | 18 | 18 |
| PLA2G4C | 8605 | phospholipase A2, group IVC (cytosolic, | 19 | 19q13.3 | 53242917 | 53305826 | 40 | 35 | 35 |
| | | calcium-independent) | | | | | | | |
| PLA2G7 | 7941 | phospholipase A2, group VII (platelet- | 6 | 6p21.2-p12 | 46780237 | 46811061 | 14 | 14 | 14 |
| | | activating factor acetylhydrolase, plasma) | | | | | | | |
| PLAT | 5327 | plasminogen activator, tissue | 8 | 8p12 | 42151393 | 42184351 | 35 | 30 | 30 |
| PLAU | 5328 | plasminogen activator, urokinase | 10 | 10q24 | 75340896 | 75347261 | 6 | 6 | 5 |
| PLAUR | 5329 | plasminogen activator, urokinase receptor | 19 | 19q13 | 48842088 | 48866342 | 19 | 16 | 16 |
| PLCB1 | 23236 | phospholipase C, beta 1 | 20 | 20p12 | 8061296 | 8813547 | 240 | 233 | 233 |
| | | (phosphoinositide-specific) | | | | | | | |
| PLCG2 | 5336 | phospholipase C, gamma 2 | 16 | 16q24.1 | 80362768 | 80549400 | 123 | 119 | 119 |
| | | (phosphatidylinositol-specific) | | | | | | | |
| PLG | 5340 | plasminogen | 6 | 6q26 | 161043273 | 161094328 | 60 | 53 | 53 |
| PLSCR1 | 5359 | phospholipid scramblase 1 | 3 | 3q23 | 147715658 | 147745186 | 20 | 19 | 19 |
| PLSCR4 | 57088 | phospholipid scramblase 4 | 3 | 3q24 | 147392816 | 147451560 | 22 | 22 | 22 |
| PLTP | 5360 | phospholipid transfer protein | 20 | 20q13.12 | 43960804 | 43974193 | 11 | 8 | 8 |
| PLUNC | 51297 | palate, lung and nasal epithelium | 20 | 20q11.2 | 31287463 | 31294773 | 4 | 4 | 4 |
| | | associated | | | | | | | |
| PON1 | 5444 | paraoxonase 1 | 7 | 7q21.3 | 94764924 | 94791780 | 32 | 30 | 30 |
| PON2 | 5445 | paraoxonase 2 | 7 | 7q21.3 | 94872110 | 94902320 | 20 | 16 | 16 |
| PON3 | 5446 | paraoxonase 3 | 7 | 7q21.3 | 94827120 | 94863623 | 11 | 10 | 10 |
| POU2AF1 | 5450 | POU class 2 associating factor 1 | 11 | 11q23.1 | 110728190 | 110755626 | 13 | 13 | 13 |
| POU2F2 | 5452 | POU class 2 homeobox 2 | 19 | 19q13.2 | 47284490 | 47328470 | 6 | 6 | 6 |
| PPARA | 5465 | peroxisome proliferator-activated | 22 | 22q12-q13.1\| | 44925163 | 45018317 | 15 | 14 | 14 |
| | | receptor alpha | | 22q13.31 | | | | | |
| PPARG | 5468 | peroxisome proliferator-activated | 3 | 3p25 | 12304349 | 12450855 | 27 | 27 | 27 |
| | | receptor gamma | | | | | | | |
| PPBP | 5473 | pro-platelet basic protein (chemokine (C- | 4 | 4q12-q13 | 75071619 | 75072764 | 9 | 6 | 6 |
| | | X-C motif) ligand 7) | | | | | | | |
| PRDX5 | 25824 | peroxiredoxin 5 | 11 | 11q13 | 63842145 | 63845859 | 5 | 5 | 5 |
| PREX1 | 57580 | phosphatidylinositol 3,4,5-trisphosphate- | 20 | 20q13.13 | 46674200 | 46877827 | 55 | 54 | 53 |
| | | dependent RAC exchanger 1 | | | | | | | |
| PRG2 | 5553 | proteoglycan 2, bone marrow (natural | 11 | 11q12 | 56911410 | 56914706 | 6 | 6 | 6 |
| | | killer cell activator, eosinophil granule | | | | | | | |
| | | major basic protein) | | | | | | | |
| PRG3 | 10394 | proteoglycan 3 | 11 | 11q12 | 56900819 | 56905199 | 5 | 5 | 5 |
| PRKAR1A | 5573 | protein kinase, cAMP-dependent, | 17 | 17q23-q24 | 64019705 | 64040506 | 6 | 6 | 6 |
| | | regulatory, type I, alpha (tissue specific | | | | | | | |
| | | extinguisher 1) | | | | | | | |
| PRKCA | 5578 | protein kinase C, alpha | 17 | 17q22-q23.2 | 61729388 | 62237324 | 153 | 147 | 146 |
| PRKCB1 | 5579 | protein kinase C, beta 1 | 16 | 16p11.2 | 23754823 | 24139063 | 103 | 101 | 101 |
| PRLR | 5618 | prolactin receptor | 5 | 5p13.2 | 35099985 | 35266334 | 39 | 39 | 39 |
| PROC | 5624 | protein C (inactivator of coagulation | 2 | 2q13-q14 | 127892487 | 127903288 | 25 | 23 | 23 |
| | | factors Va and VIIIa) | | | | | | | |
| PROCR | 10544 | protein C receptor, endothelial (EPCR) | 20 | 20q11.2 | 33223435 | 33228826 | 6 | 5 | 5 |
| PROK2 | 60675 | prokineticin 2 | 3 | 3p13 | 71903497 | 71916902 | 10 | 10 | 10 |
| PROS1 | 5627 | protein S (alpha) | 3 | 3q11.2 | 95074647 | 95175395 | 17 | 17 | 17 |
| PROZ | 8858 | protein Z, vitamin K-dependent plasma | 13 | 13q34 | 112860969 | 112874695 | 22 | 20 | 20 |
| | | glycoprotein | | | | | | | |
| PTAFR | 5724 | platelet-activating factor receptor | 1 | 1p35-p34.3 | 28348425 | 28375778 | 2 | 2 | 2 |
| PTEN | 5728 | phosphatase and tensin homolog (mutated | 10 | 10q23.3 | 89613175 | 89718512 | 10 | 9 | 9 |
| | | in multiple advanced cancers 1) | | | | | | | |
| PTGDR | 5729 | prostaglandin D2 receptor (DP) | 14 | 14q22.1 | 51804181 | 51813192 | 13 | 13 | 13 |
| PTGER3 | 5733 | prostaglandin E receptor 3 (subtype EP3) | 1 | 1p31.2 | 71090624 | 71286079 | 47 | 47 | 47 |
| PTGER4 | 5734 | prostaglandin E receptor 4 (subtype EP4) | 5 | 5p13.1 | 40715789 | 40729594 | 7 | 6 | 6 |
| PTGES | 9536 | prostaglandin E synthase | 9 | 9q34.3 | 131540433 | 131555165 | 9 | 9 | 9 |
| PTGIS | 5740 | prostaglandin I2 (prostacyclin) synthase | 20 | 20q13.13 | 47553818 | 47618114 | 17 | 14 | 14 |
| PTGS2 | 5743 | prostaglandin-endoperoxide synthase 2 | 1 | 1q25.2-q25.3 | 184907592 | 184916179 | 8 | 7 | 6 |
| | | (prostaglandin G/H synthase and | | | | | | | |
| | | cyclooxygenase) | | | | | | | |
| PTPNS1L | 23755 | protein tyrosine phosphatase, non- | 22 | 22q12.2 | 29268342 | 29271028 | 4 | 4 | 4 |
| | | receptor type substrate 1-like | | | | | | | |
| PTPRA | 5786 | protein tyrosine phosphatase, receptor | 20 | 20p13 | 2792841 | 2967315 | 19 | 19 | 19 |
| | | type, A | | | | | | | |
| PTX3 | 5806 | pentraxin-related gene, rapidly induced | 3 | 3q25 | 158637301 | 158644071 | 9 | 9 | 9 |
| | | by IL-1 beta | | | | | | | |
| RAC1 | 5879 | ras-related C3 botulinum toxin substrate 1 | 7 | 7p22 | 6380651 | 6410123 | 20 | 18 | 18 |
| | | (rho family, small GTP binding protein | | | | | | | |
| | | Rac1) | | | | | | | |
| REG3A | 5068 | regenerating islet-derived 3 alpha | 2 | 2p12 | 79237641 | 79240387 | 3 | 2 | 2 |
| REL | 5966 | v-rel reticuloendotheliosis viral oncogene | 2 | 2p13-p12 | 60962256 | 61003682 | 7 | 6 | 6 |
| | | homolog (avian) | | | | | | | |
| RELA | 5970 | v-rel reticuloendotheliosis viral oncogene | 11 | 11q13 | 65178393 | 65186951 | 5 | 5 | 5 |
| | | homolog A, nuclear factor of kappa light | | | | | | | |
| | | polypeptide gene enhancer in B-cells 3, | | | | | | | |
| | | p65 (avian) | | | | | | | |
| RELB | 5971 | v-rel reticuloendotheliosis viral oncogene | 19 | 19q13.32 | 50196552 | 50233292 | 4 | 4 | 4 |
| | | homolog B, nuclear factor of kappa light | | | | | | | |
| | | polypeptide gene enhancer in B-cells 3 | | | | | | | |
| | | (avian) | | | | | | | |
| RFXANK | 8625 | regulatory factor X-associated ankyrin- | 19 | 19p12 | 19164008 | 19173678 | 5 | 4 | 4 |
| | | containing protein | | | | | | | |
| RHOB | 388 | ras homolog gene family, member B | 2 | 2p24 | 20510316 | 20512682 | 6 | 6 | 6 |
| RIPK1 | 8737 | receptor (TNFRSF)-interacting serine- | 6 | 6p25.2 | 3022057 | 3060420 | 10 | 9 | 9 |
| | | threonine kinase 1 | | | | | | | |
| RIPK2 | 8767 | receptor-interacting serine-threonine | 8 | 8q21 | 90839110 | 90872433 | 9 | 8 | 8 |
| | | kinase 2 | | | | | | | |
| RNASE7 | 84659 | ribonuclease, RNase A family, 7 | 14 | 14q11.2 | 20580225 | 20582232 | 17 | 17 | 17 |
| RPS6KA4 | 8986 | ribosomal protein S6 kinase, 90 kDa, | 11 | 11q11-q13 | 63883201 | 63896263 | 3 | 3 | 3 |
| | | polypeptide 4 | | | | | | | |
| S100A12 | 6283 | S100 calcium binding protein A12 | 1 | 1q21 | 151612808 | 151614699 | 5 | 5 | 5 |
| S100A8 | 6279 | S100 calcium binding protein A8 | 1 | 1q21 | 151629132 | 151630173 | 4 | 4 | 4 |
| S100B | 6285 | S100 calcium binding protein B | 21 | 21q22.3 | 46842959 | 46849463 | 12 | 11 | 11 |
| SAA2 | 6289 | serum amyloid A2 | 11 | 11p15.1-p14 | 18223365 | 18226744 | 13 | 12 | 12 |
| SAA3P | 6290 | serum amyloid A3 pseudogene | 11 | 11p15.1-p14 | 18090610 | 18094695 | 4 | 3 | 3 |
| SAA4 | 6291 | serum amyloid A4, constitutive | 11 | 11p15.1-p14 | 18209479 | 18214910 | 12 | 11 | 11 |
| SCUBE1 | 80274 | signal peptide, CUB domain, EGF-like 1 | 22 | 22q13 | 41929174 | 42069299 | 85 | 83 | 83 |
| SCYE1 | 9255 | small inducible cytokine subfamily E, | 4 | 4q24 | 107457124 | 107489021 | 5 | 4 | 4 |
| | | member 1 (endothelial monocyte- | | | | | | | |
| | | activating) | | | | | | | |
| SELE | 6401 | selectin E (endothelial adhesion molecule | 1 | 1q22-q25 | 167958406 | 167969803 | 18 | 18 | 18 |
| | | 1) | | | | | | | |
| SELL | 6402 | selectin L (lymphocyte adhesion | 1 | 1q23-q25 | 167926432 | 167947461 | 20 | 20 | 20 |
| | | molecule 1) | | | | | | | |
| SELP | 6403 | selectin P (granule membrane protein | 1 | 1q22-q25 | 167824711 | 167866001 | 79 | 72 | 72 |
| | | 140 kDa, antigen CD62) | | | | | | | |
| SELPLG | 6404 | selectin P ligand | 12 | 12q24 | 107539800 | 107551799 | 12 | 10 | 10 |
| SEMA7A | 8482 | semaphorin 7A, GPI membrane anchor | 15 | 15q22.3-q23 | 72489376 | 72513329 | 14 | 14 | 14 |
| | | (John Milton Hagen blood group) | | | | | | | |
| SERPINA1 | 5265 | serpin peptidase inhibitor, clade A (alpha- | 14 | 14q32.1 | 93914451 | 93926782 | 26 | 25 | 25 |
| | | 1 antiproteinase, antitrypsin), member 1 | | | | | | | |
| SERPINA10 | 51156 | serpin peptidase inhibitor, clade A (alpha- | 14 | 14q32.13 | 93819403 | 93829349 | 23 | 23 | 23 |
| | | 1 antiproteinase, antitrypsin), member 10 | | | | | | | |
| SERPINA3 | 12 | serpin peptidase inhibitor, clade A (alpha- | 14 | 14q32.1 | 94148467 | 94160143 | 14 | 14 | 14 |
| | | 1 antiproteinase, antitrypsin), member 3 | | | | | | | |
| SERPINA5 | 5104 | serpin peptidase inhibitor, clade A (alpha- | 14 | 14q32.1 | 94117564 | 94129205 | 26 | 24 | 24 |
| | | 1 antiproteinase, antitrypsin), member 5 | | | | | | | |
| SERPINC1 | 462 | serpin peptidase inhibitor, clade C | 1 | 1q23-q25.1 | 172139565 | 172153096 | 16 | 14 | 14 |
| | | (antithrombin), member 1 | | | | | | | |
| SERPIND1 | 3053 | serpin peptidase inhibitor, clade D | 22 | 22q11.2\|22q11.21 | 19458383 | 19472008 | 6 | 6 | 6 |
| | | (heparin cofactor), member 1 | | | | | | | |
| SERPINE1 | 5054 | serpin peptidase inhibitor, clade E (nexin, | 7 | 7q21.3-q22 | 100557172 | 100569026 | 19 | 19 | 19 |
| | | plasminogen activator inhibitor type 1), | | | | | | | |
| | | member 1 | | | | | | | |
| SERPINE2 | 5270 | serpin peptidase inhibitor, clade E (nexin, | 2 | 2q33-q35 | 224548118 | 224612237 | 20 | 20 | 20 |
| | | plasminogen activator inhibitor type 1), | | | | | | | |
| | | member 2 | | | | | | | |
| SERPINF2 | 5345 | serpin peptidase inhibitor, clade F (alpha- | 17 | 17p13 | 1592880 | 1605309 | 13 | 13 | 13 |
| | | 2 antiplasmin, pigment epithelium | | | | | | | |
| | | derived factor), member 2 | | | | | | | |
| SERPING1 | 710 | serpin peptidase inhibitor, clade G (C1 | 11 | 11q12-q13.1 | 57121603 | 57138902 | 8 | 8 | 8 |
| | | inhibitor), member 1, (angioedema, | | | | | | | |
| | | hereditary) | | | | | | | |
| SFTPA1B | 6435 | surfactant, pulmonary-associated protein | 10 | | 81360664 | 81363921 | 4 | 4 | 4 |
| | | A1B | | | | | | | |
| SFTPA2B | 6436 | surfactant, pulmonary-associated protein | 10 | | 81305573 | 81310114 | 1 | 1 | 1 |
| | | A2B | | | | | | | |
| SFTPD | 6441 | surfactant, pulmonary-associated protein D | 10 | 10q22.2-q23.1 | 81687476 | 81698841 | 16 | 16 | 16 |
| SIGIRR | 59307 | single immunoglobulin and toll- | 11 | 11p15.5 | 395716 | 407397 | 3 | 2 | 2 |
| | | interleukin 1 receptor (TIR) domain | | | | | | | |
| SIGLEC1 | 6614 | sialic acid binding Ig-like lectin 1, | 20 | 20p13 | 3615617 | 3635775 | 20 | 20 | 20 |
| | | sialoadhesin | | | | | | | |
| SLA2 | 84174 | Src-like-adaptor 2 | 20 | 20q11.23 | 34674336 | 34707972 | 5 | 5 | 5 |
| SOCS1 | 8651 | suppressor of cytokine signaling 1 | 16 | 16p13.13 | 11255775 | 11257540 | 5 | 5 | 5 |
| SOCS2 | 8835 | suppressor of cytokine signaling 2 | 12 | 12q | 92487729 | 92494109 | 5 | 5 | 5 |
| SOCS3 | 9021 | suppressor of cytokine signaling 3 | 17 | 17q25.3 | 73864454 | 73867753 | 5 | 5 | 5 |
| SOCS4 | 122809 | suppressor of cytokine signaling 4 | 14 | 14q22.1 | 54563594 | 54585960 | 3 | 3 | 3 |
| SOCS5 | 9655 | suppressor of cytokine signaling 5 | 2 | 2p21 | 46779603 | 46843431 | 14 | 14 | 14 |
| SOCS6 | 9306 | suppressor of cytokine signaling 6 | 18 | 18q22.2 | 66107117 | 66148414 | 14 | 14 | 14 |
| SOD1 | 6647 | superoxide dismutase 1, soluble | 21 | 21q22.1\|21q22.11 | 31953806 | 31963115 | 5 | 5 | 5 |
| | | (amyotrophic lateral sclerosis 1 (adult)) | | | | | | | |
| SOD2 | 6648 | superoxide dismutase 2, mitochondrial | 6 | 6q25.3 | 160020138 | 160034343 | 7 | 7 | 7 |
| SPACA3 | 124912 | sperm acrosome associated 3 | 17 | 17q11.2 | 28342995 | 28349005 | 25 | 24 | 24 |
| SPARC | 6678 | secreted protein, acidic, cysteine-rich | 5 | 5q31.3-q32 | 151021201 | 151046710 | 17 | 17 | 17 |
| | | (osteonectin) | | | | | | | |
| SPINK4 | 27290 | serine peptidase inhibitor, Kazal type 4 | 9 | 9p13.3 | 33230196 | 33238565 | 6 | 6 | 6 |
| SPINK5 | 11005 | serine peptidase inhibitor, Kazal type 5 | 5 | 5q32 | 147423759 | 147497120 | 16 | 15 | 15 |
| SPN | 6693 | sialophorin (leukosialin, CD43) | 16 | 16p11.2 | 29581801 | 29589329 | 3 | 3 | 3 |
| SPP1 | 6696 | secreted phosphoprotein 1 (osteopontin, | 4 | 4q22.1 | 89115826 | 89123587 | 6 | 5 | 5 |
| | | bone sialoprotein I, early T-lymphocyte | | | | | | | |
| | | activation 1) | | | | | | | |
| SRC | 6714 | v-src sarcoma (Schmidt-Ruppin A-2) | 20 | 20q12-q13 | 35406502 | 35467235 | 15 | 15 | 15 |
| | | viral oncogene homolog (avian) | | | | | | | |
| SRF | 6722 | serum response factor (c-fos serum | 6 | 6p21.1 | 43246898 | 43257222 | 6 | 6 | 6 |
| | | response element-binding transcription | | | | | | | |
| | | factor) | | | | | | | |
| STAB1 | 23166 | stabilin 1 | 3 | 3p21.1 | 52504396 | 52533551 | 12 | 11 | 11 |
| STAT2 | 6773 | signal transducer and activator of | 12 | 12q13.3 | 55021648 | 55040176 | 3 | 3 | 3 |
| | | transcription 2, 113 kDa | | | | | | | |
| STAT3 | 6774 | signal transducer and activator of | 17 | 17q21.31 | 37718869 | 37794039 | 11 | 11 | 11 |
| | | transcription 3 (acute-phase response | | | | | | | |
| | | factor) | | | | | | | |
| STAT4 | 6775 | signal transducer and activator of | 2 | 2q32.2-q32.3 | 191602551 | 191724170 | 40 | 39 | 39 |
| | | transcription 4 | | | | | | | |
| STAT5A | 6776 | signal transducer and activator of | 17 | 17q11.2 | 37693091 | 37717484 | 3 | 3 | 3 |
| | | transcription 5A | | | | | | | |
| STAT5B | 6777 | signal transducer and activator of | 17 | 17q11.2 | 37604721 | 37681950 | 4 | 4 | 4 |
| | | transcription 5B | | | | | | | |
| STAT6 | 6778 | signal transducer and activator of | 12 | 12q13 | 55775458 | 55791428 | 10 | 8 | 8 |
| | | transcription 6, interleukin-4 induced | | | | | | | |
| STX4 | 6810 | syntaxin 4 | 16 | 16p11.2 | 30952404 | 30958986 | 1 | 1 | 1 |
| SYK | 6850 | spleen tyrosine kinase | 9 | 9q22 | 92603891 | 92698305 | 71 | 69 | 69 |
| TACR1 | 6869 | tachykinin receptor 1 | 2 | 2p12 | 75129738 | 75279781 | 50 | 49 | 49 |
| TANK | 10010 | TRAF family member-associated NFKB | 2 | 2q24-q31 | 161701712 | 161800928 | 10 | 9 | 9 |
| | | activator | | | | | | | |
| TBK1 | 29110 | TANK-binding kinase 1 | 12 | 12q14.1 | 63132204 | 63182158 | 5 | 5 | 5 |
| TBX21 | 30009 | T-box 21 | 17 | 17q21.32 | 43165609 | 43178484 | 7 | 6 | 6 |
| TBXA2R | 6915 | thromboxane A2 receptor | 19 | 19p13.3 | 3545504 | 3557658 | 13 | 10 | 10 |
| TBXAS1 | 6916 | thromboxane A synthase 1 (platelet, | 7 | 7q34-q35 | 139175421 | 139366471 | 59 | 59 | 59 |
| | | cytochrome P450, family 5, subfamily A) | | | | | | | |
| TEK | 7010 | TEK tyrosine kinase, endothelial (venous | 9 | 9p21 | 27099286 | 27220172 | 81 | 81 | 81 |
| | | malformations, multiple cutaneous and | | | | | | | |
| | | mucosal) | | | | | | | |
| TFPI | 7035 | tissue factor pathway inhibitor | 2 | 2q32 | 188037202 | 188127464 | 42 | 37 | 37 |
| | | (lipoprotein-associated coagulation | | | | | | | |
| | | inhibitor) | | | | | | | |
| TGFB1 | 7040 | transforming growth factor, beta 1 | 19 | 19q13.2\|19q13.1 | 46528491 | 46551656 | 10 | 10 | 10 |
| TGFB2 | 7042 | transforming growth factor, beta 2 | 1 | 1q41 | 216586491 | 216681596 | 26 | 25 | 25 |
| TGFB3 | 7043 | transforming growth factor, beta 3 | 14 | 14q24 | 75494195 | 75517242 | 8 | 8 | 8 |
| TGFBR1 | 7046 | transforming growth factor, beta receptor | 9 | 9q22 | 100907233 | 100956295 | 7 | 7 | 7 |
| | | I (activin A receptor type II-like kinase, | | | | | | | |
| | | 53 kDa) | | | | | | | |
| TGFBR2 | 7048 | transforming growth factor, beta receptor | 3 | 3p22 | 30622998 | 30710638 | 42 | 41 | 41 |
| | | II (70/80 kDa) | | | | | | | |
| THBD | 7056 | thrombomodulin | 20 | 20p11.2 | 22974270 | 22978301 | 16 | 14 | 14 |
| THBS1 | 7057 | thrombospondin 1 | 15 | 15q15 | 37660572 | 37676960 | 22 | 22 | 22 |
| THBS4 | 7060 | thrombospondin 4 | 5 | 5q13 | 79366747 | 79414866 | 16 | 15 | 15 |
| TICAM1 | 148022 | toll-like receptor adaptor molecule 1 | 19 | 19p13.3 | 4766992 | 4769451 | 4 | 3 | 3 |
| TIMP1 | 7076 | TIMP metallopeptidase inhibitor 1 | X | Xp11.3-p11.23 | 47326634 | 47331134 | 3 | 3 | 3 |
| TIRAP | 114609 | toll-interleukin 1 receptor (TIR) domain | 11 | 11q24.2 | 125658192 | 125670038 | 14 | 13 | 13 |
| | | containing adaptor protein | | | | | | | |
| TLR1 | 7096 | toll-like receptor 1 | 4 | 4p14 | 38474271 | 38482807 | 7 | 5 | 5 |
| TLR10 | 81793 | toll-like receptor 10 | 4 | 4p14 | 38450629 | 38460984 | 16 | 13 | 13 |
| TLR2 | 7097 | toll-like receptor 2 | 4 | 4q32 | 154824891 | 154846693 | 12 | 9 | 9 |
| TLR3 | 7098 | toll-like receptor 3 | 4 | 4q35 | 187227303 | 187243246 | 10 | 10 | 10 |
| TLR4 | 7099 | toll-like receptor 4 | 9 | 9q33.1 | 119506431 | 119519589 | 14 | 13 | 13 |
| TLR5 | 7100 | toll-like receptor 5 | 1 | 1q41-q42 | 221350207 | 221383247 | 10 | 9 | 9 |
| TLR6 | 10333 | toll-like receptor 6 | 4 | 4p14 | 38504803 | 38507555 | 6 | 5 | 5 |
| TLR7 | 51284 | toll-like receptor 7 | X | Xp22.3 | 12795123 | 12818401 | 18 | 16 | 16 |
| TLR8 | 51311 | toll-like receptor 8 | X | Xp22 | 12834679 | 12851209 | 15 | 13 | 13 |
| TLR9 | 54106 | toll-like receptor 9 | 3 | 3p21.3 | 52230138 | 52235219 | 6 | 4 | 4 |
| TMED7 | 51014 | transmembrane emp24 protein transport | 5 | 5q22.3 | 114977102 | 114989595 | 7 | 7 | 7 |
| | | domain containing 7 | | | | | | | |
| TNF | 7124 | tumor necrosis factor (TNF superfamily, | 6 | 6p21.3 | 31651329 | 31654091 | 19 | 15 | 15 |
| | | member 2) | | | | | | | |
| TNFAIP3 | 7128 | tumor necrosis factor, alpha-induced | 6 | 6q23 | 138230274 | 138246142 | 6 | 6 | 6 |
| | | protein 3 | | | | | | | |
| TNFAIP8 | 25816 | tumor necrosis factor, alpha-induced | 5 | 5q23.1 | 118632317 | 118758193 | 6 | 6 | 6 |
| | | protein 8 | | | | | | | |
| TNFRSF10A | 8797 | tumor necrosis factor receptor | 8 | 8p21 | 23104915 | 23138584 | 19 | 18 | 18 |
| | | superfamily, member 10a | | | | | | | |
| TNFRSF10C | 8794 | tumor necrosis factor receptor | 8 | 8p22-p21 | 23016379 | 23030895 | 11 | 9 | 9 |
| | | superfamily, member 10c, decoy without | | | | | | | |
| | | an intracellular domain | | | | | | | |
| TNFRSF1A | 7132 | tumor necrosis factor receptor | 12 | 12p13.2 | 6308184 | 6321522 | 10 | 9 | 9 |
| | | superfamily, member 1A | | | | | | | |
| TNFRSF1B | 7133 | tumor necrosis factor receptor | 1 | 1p36.22 | 12149647 | 12191864 | 26 | 24 | 24 |
| | | superfamily, member 1B | | | | | | | |
| TNFRSF21 | 27242 | tumor necrosis factor receptor | 6 | 6p21.1 | 47307227 | 47385639 | 28 | 26 | 26 |
| | | superfamily, member 21 | | | | | | | |
| TNFRSF25 | 8718 | tumor necrosis factor receptor | 1 | 1p36.2 | 6443798 | 6448842 | 6 | 5 | 5 |
| | | superfamily, member 25 | | | | | | | |
| TNFRSF8 | 943 | tumor necrosis factor receptor | 1 | 1p36 | 12046021 | 12126851 | 27 | 27 | 27 |
| | | superfamily, member 8 | | | | | | | |
| TNFRSF9 | 3604 | tumor necrosis factor receptor | 1 | 1p36 | 7902494 | 7923474 | 5 | 5 | 5 |
| | | superfamily, member 9 | | | | | | | |
| TNFSF15 | 9966 | tumor necrosis factor (ligand) | 9 | 9q32 | 116591421 | 116608229 | 8 | 8 | 8 |
| | | superfamily, member 15 | | | | | | | |
| TNIP1 | 10318 | TNFAIP3 interacting protein 1 | 5 | 5q32-q33.1 | 150389699 | 150441190 | 31 | 29 | 29 |
| TOLLIP | 54472 | toll interacting protein | 11 | 11p15.5 | 1252177 | 1287415 | 10 | 10 | 10 |
| TP53 | 7157 | tumor protein p53 | 17 | 17p13.1 | 7512445 | 7531642 | 5 | 5 | 5 |
| TPST1 | 8460 | tyrosylprotein sulfotransferase 1 | 7 | 7q11.21 | 65307750 | 65462865 | 8 | 8 | 8 |
| TRADD | 8717 | TNFRSF1A-associated via death domain | 16 | 16q22 | 65745589 | 65751313 | 3 | 1 | 1 |
| TRAF1 | 7185 | TNF receptor-associated factor 1 | 9 | 9q33-q34 | 122704493 | 122728994 | 6 | 6 | 6 |
| TRAF2 | 7186 | TNF receptor-associated factor 2 | 9 | 9q34 | 138900786 | 138940888 | 14 | 14 | 14 |
| TRAF3 | 7187 | TNF receptor-associated factor 3 | 14 | 14q32.32 | 102313569 | 102442381 | 16 | 16 | 16 |
| TRAF5 | 7188 | TNF receptor-associated factor 5 | 1 | 1q32 | 209566580 | 209614911 | 6 | 6 | 6 |
| TRAF6 | 7189 | TNF receptor-associated factor 6 | 11 | 11p12 | 36467302 | 36488398 | 11 | 11 | 11 |
| TREM1 | 54210 | triggering receptor expressed on myeloid | 6 | 6p21.1 | 41351690 | 41362435 | 11 | 10 | 10 |
| | | cells 1 | | | | | | | |
| TTN | 7273 | titin | 2 | 2q31 | 179098962 | 179380395 | 36 | 36 | 36 |
| TXN | 7295 | thioredoxin | 9 | 9q31 | 112046131 | 112058599 | 23 | 23 | 23 |
| TYK2 | 7297 | tyrosine kinase 2 | 19 | 19p13.2 | 10322209 | 10352211 | 11 | 9 | 9 |
| TYMP | 1890 | thymidine phosphorylase | 22 | 22q13\|22q13.33 | 49311047 | 49315321 | 8 | 8 | 8 |
| UBE2D3 | 7323 | ubiquitin-conjugating enzyme E2D 3 | 4 | 4q24 | 103936217 | 104009473 | 10 | 10 | 10 |
| | | (UBC4/5 homolog, yeast) | | | | | | | |
| UBTF | 7343 | upstream binding transcription factor, | 17 | 17q21.3 | 39637927 | 39653776 | 3 | 3 | 3 |
| | | RNA polymerase I | | | | | | | |
| USF1 | 7391 | upstream transcription factor 1 | 1 | 1q22-q23 | 159275665 | 159282381 | 9 | 9 | 9 |
| VASP | 7408 | vasodilator-stimulated phosphoprotein | 19 | 19q13.2-q13.3 | 50702528 | 50722081 | 11 | 11 | 11 |
| VCAM1 | 7412 | vascular cell adhesion molecule 1 | 1 | 1p32-p31 | 100957885 | 100977189 | 13 | 13 | 13 |
| VCL | 7414 | vinculin | 10 | 10q22.2 | 75427878 | 75549924 | 14 | 12 | 12 |
| VEGFA | 7422 | vascular endothelial growth factor A | 6 | 6p12 | 43845931 | 43862202 | 15 | 15 | 15 |
| VEGFB | 7423 | vascular endothelial growth factor B | 11 | 11q13 | 63758842 | 63762835 | 5 | 4 | 4 |
| VEGFC | 7424 | vascular endothelial growth factor C | 4 | 4q34.3 | 177841685 | 177950889 | 11 | 11 | 11 |
| VISA | 57506 | virus-induced signaling adapter | 20 | 20p13 | 3775484 | 3795973 | 9 | 9 | 9 |
| VKORC1 | 79001 | vitamin K epoxide reductase complex, | 16 | 16p11.2 | 31009676 | 31013777 | 2 | 2 | 2 |
| | | subunit 1 | | | | | | | |
| VPS45 | 11311 | vacuolar protein sorting 45 homolog (S. cerevisiae) | 1 | 1q21.2 | 148305966 | 148384129 | 4 | 4 | 4 |
| VTN | 7448 | vitronectin | 17 | 17q11 | 23718425 | 23721500 | 6 | 5 | 5 |
| VWF | 7450 | von Willebrand factor | 12 | 12p13.3 | 5928301 | 6104097 | 155 | 148 | 148 |
| XCL1 | 6375 | chemokine (C motif) ligand 1 | 1 | 1q23 | 166812480 | 166817939 | 8 | 8 | 8 |
| XCR1 | 2829 | chemokine (C motif) receptor 1 | 3 | 3p21.3-p21.1\| | 46037295 | 46043983 | 4 | 3 | 3 |
| | | | | 3p21.3 | | | | | |
| YARS | 8565 | tyrosyl-tRNA synthetase | 1 | 1p35.1 | 33013427 | 33056220 | 7 | 7 | 7 |
| YY1 | 7528 | YY1 transcription factor | 14 | 14q | 99774855 | 99814557 | 4 | 4 | 4 |
| | | | | | | | | 12283 | 12226 |

### Genotyping and Quality Control

Leukocyte genomic DNA was extracted, quantified, and diluted to the appropriate concentration for Illumina Infinium iSelect genotyping on all samples collected. Controls included 2% sample replicates and a CEPH trio for quality control. In addition, case and control DNA sample addresses were randomly assigned across both the 96-well plate as well as the 12-address iSelect BeadChip, insuring approximately equal numbers of case and control DNA samples by each strata to avoid potential plate and chip effects, respectively. Genotyping results from high-quality control DNA (SNP call rate 95%) was used to generate a cluster algorithm.

### Statistical Analyses

The primary outcome was VTE status, a binary measure. The covariates were age at interview or blood sample collection, sex, stroke and/or MI status, and state of residence (Table 2). To adjust for population stratification, the multidimensional scaling (MDS) analysis option in PLINK v 1.07 was performed to identify outliers in the population (Purcell et al., American Journal of Human Genetics., 81:559-75 (2007)) using the ancestry informative markers. Association between each SNP and VTE were tested for using unconditional logistic regression, adjusting for age, sex, stroke/MI status, and state of residence. The analyses were corrected for multiple comparisons using an extension of false discovery rates (Benjamini et al., Behavioural Brain Research., 125:279-84 (2001) and Storey et al., Proc. Nat'l. Acad. Sci. USA, 100:9440-5 (2003)). The false discovery rate was an analogue measure of the p-value that takes into account the number of statistical tests and estimates the expected proportion of false positive tests incurred when a particular SNP is significant. All analyses were performed using PLINK v 1.07 Purcell et al., American Journal of Human Genetics., 81:559-75 (2007)). Quantile-quantile (QQ) plots of observed −log₁₀ p-values for VTE association versus the expected −log₁₀ p-values under the null hypothesis of no association were generated to display the potential significant associations (Wakefield, International Journal of Epidemiology, 37:641-53 (2008), and to calculate the genomic inflation factor as a check for over dispersion of the test statistics (Clayton et al., Nature Genetics, 37:1243-6 (2005). Penalized logistic regression models were used to determine possible interaction between the statistically significant SNPs (Park and Hastie, Biostatistics, 9:30-50 (2008)).

TABLE 2
Demographic and Clinical Characteristics by Case-Control Status

| | Case | Control | |
|---|---|---|---|
| Characteristic | n = 1488 | n = 1439 | P-value |
| Patient age, mean ± SD, years | 54.7 ± 16.3 | 55.5 ± 15.7 | 0.1796 |
| Female, n (%) | 751 (50.5) | 754 (52.4) | 0.2970 |
| Stroke or myocardial | 283 (19.0) | 149 (10.4) | <0.0001 |
| infarction, n (%) | | | |
| State of residence, n (%) | | | <0.0001 |
| Minnesota | 619 (41.6) | 795 (55.2) | |
| Other States | 869 (58.4) | 644 (44.8) | |

Population attributable risk (PAR) was estimated for each genotype, which defines the percentage of the total risk for VTE due to genetic effect of that particular genotype (Cole and MacMahon, British Journal of Preventive & Social Medicine, 25:242-4 (1971):

$$PAR = \frac{p \times ( OR - 1 )}{p \times ( OR - 1 ) + 1}$$

where p is the prevalence of risk genotype associated with VTE among control subjects, and OR is the odds ratio associated with risk genotype. Odds ratios from the dominant genetic model adjusted for age at blood draw, gender, MI/stroke status, and state of residence were used. The group PAR was calculated as:

$$1 - {\prod }_{i = 1}^{n} ( 1 - {PAR}_{i} )$$

on the basis of the individual PARᵢ of each associated genotype assuming a dominant genetic model and no multiplicative interaction among the genotypes (i.e., assuming independence between SNPs). The joint PAR was calculated as:

$$1 - {\sum }_{j} \frac{{ρ}_{j}}{{OR}_{j}}$$

assuming multiple loci, ρ₁ as the fraction of cases for each associated genotype, and ORⱼ as the individual OR for each associated SNP or genotype calculated under the full logistic regression model (Bruzzi et al., American Journal of Epidemiology, 122:904-14 (1985)). The PAR was calculated, both unadjusted and adjusted, assuming a dominant genetic model for the genotypes. The AttribRisk Splus function (glm function, binomial error) was used to estimate this PAR, unadjusted and adjusted for covariates using jackknife estimates for the standard error (Kahn et al., Technical Report Series No 54 Department of Health Science Research, Mayo Clinic Rochester, Minn., 1994).

### Results

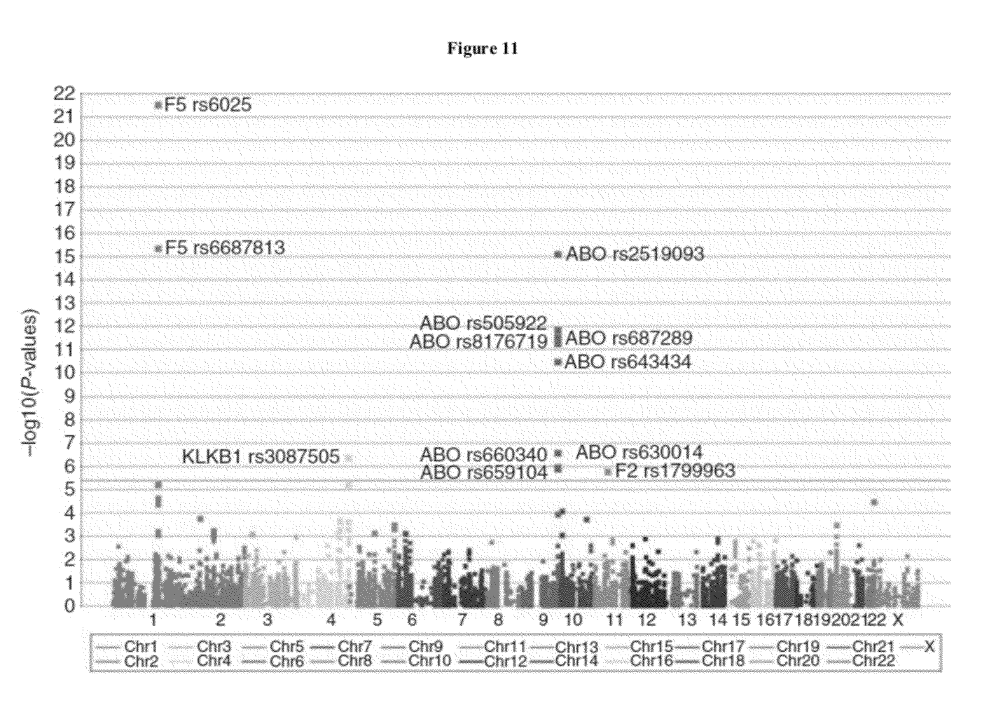

Of the 3131 unique subjects recruited, 204 were excluded due to the following overlapping reasons: study exclusion criteria (n=78), genotype issues (n=40; 22 failed genotyping and 18 had a genotype call rate <95%), mislabeled samples (n=32), and non-European (i.e., African- or Asian-American) race (n=20). Thirty-four subjects were removed due to relatedness using IBS clustering in PLINK. Multidimensional scaling plots showed two subjects with race discrepancy (FIG. 10). After removal of these subjects, no evidence of population stratification was found. After these exclusions, a total of 2,927 individuals (1,488 VTE subjects [51%], 1,439 controls; 51% women) were included in the analyses. The study population demographic and clinical characteristics by case status are presented in Table 2. Among the cases, the distribution of symptomatic VTE by event type was DVT only (n=744; 50%), PE only (n=390; 26.2%), and both DVT and PE (n=354; 23.8%).

Of the 14,612 SNPs submitted to Illumina from 764 genes within the anticoagulant, procoagulant, fibrinolytic and innate immunity pathways (Table 3), 1,585 SNPs (covering 1,100 LD bins) failed manufacture. Of the remaining 13,027 successfully manufactured SNPs (Illumina) and 4 SNPs on TaqMan, 735 SNPs were excluded due to poor performance (n=554), a MAF <0.005 (n=127) or a call rate <0.95 (n=54), leaving 12,296 SNPs (covering 10,456 bins) for the association analysis. Prior to exclusion, concordance between the genotype results for Factor V Leiden (F5 rs6025) and data obtained clinically for cases was 100% (218/218) between positive cases and 99% (446/450) between negative cases.

TABLE 3
Number of LD Bins after Illumina Infinium Custom Genotyping SNP
Selection, Design, Manufacture and Assay by Pathway

| Pathway | After | After | After | After | % Total |
|---|---|---|---|---|---|
| (# of Genes)* | Selection | Design | Manufacture | QC | Lost |
| Anticoagulant (16) | 234 | 232 | 214 | 210 | 10.3 |
| Procoagulant (75) | 1516 | 1466 | 1354 | 1300 | 14.2 |
| Fibrinolytic (23) | 435 | 411 | 373 | 356 | 18.2 |
| Innate | 10373 | 9964 | 9032 | 8590 | 17.2 |
| Immunity (635) | | | | | |

*15 genes out of 764 did not have SNPs after quality control (QC)

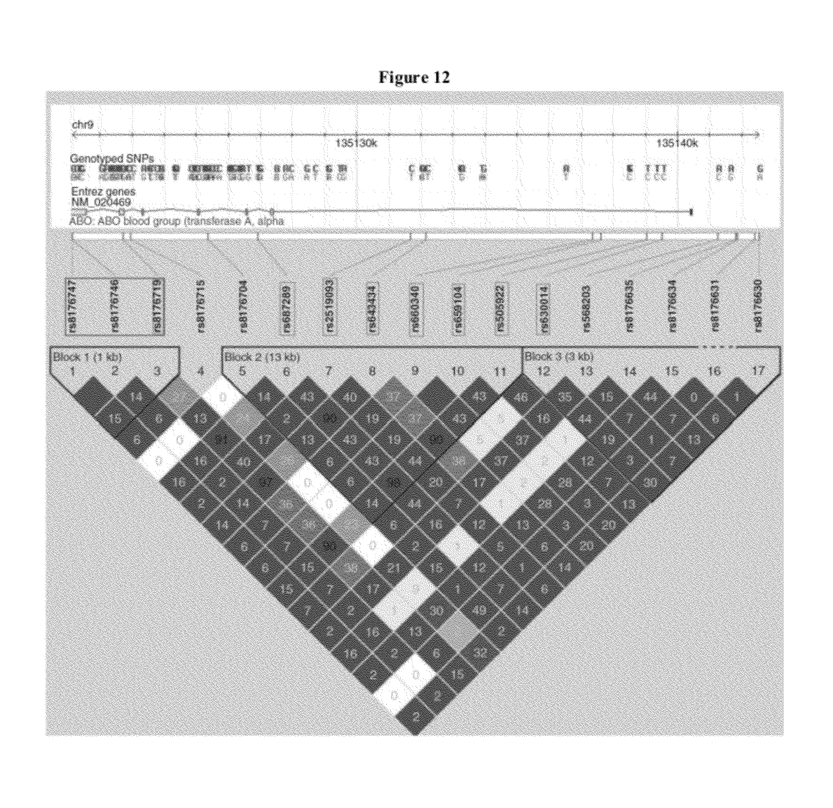

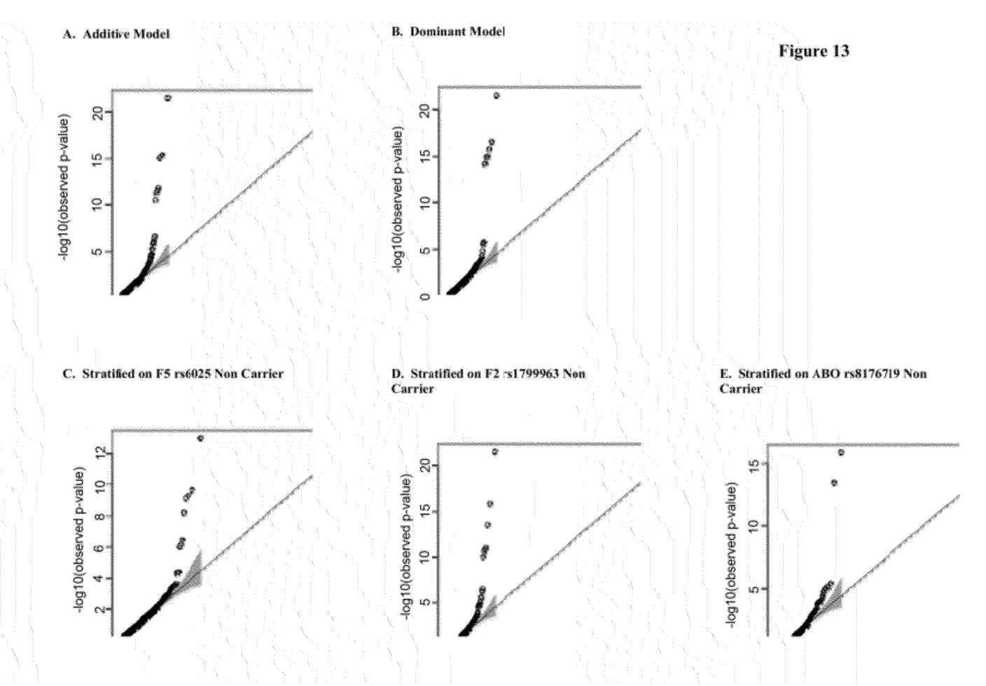

Using an additive genetic model and adjusting for age, gender, stroke/MI status and state of residence, and a false discovery rate (q-value)<0.05, one or more SNPs within ABO, F2, F5, F11, KLKB1, SELP and SCUBE1 were significantly associated with VTE (Table 4 and FIG. 11). The association between VTE and Factor V Leiden (F5 rs6025, OR=3.40, p-value=3.07×10⁻²²) was confirmed, but after controlling for F5 rs6025, F5 rs6687813 was not associated with VTE. The association between VTE and ABO non-O blood type (ABO rs8176719, OR=1.47, p-value=5.68×10⁻¹²) was also confirmed, and a novel association with ABO rs2519093 (OR=1.69, p-value=8.08×10⁻¹⁶) that remained significant after controlling for non-O blood type (OR=1.52, p-value=1.35×10⁻⁶; FIG. 12) was found. An association between VTE and prothrombin G20210A (F2 rs1799963, OR=2.46, p-value=1.69×10⁻⁶) was found. An association analysis using a dominant genetic model gave similar results.

TABLE 4
Association* of Candidate Gene SNPs with Venous Thromboembolism.

| | | | Minor | | Odds | | |
|---|---|---|---|---|---|---|---|
| SNP | Gene | Chromosome | Allele | N | Ratio | P-value | Q-value |
| rs6025 | F5 | 1 | A | 2927 | 3.40 | 3.07E−22 | 3.77E−18 |
| rs6687813 | F5 | 1 | A | 2926 | 2.13 | 4.66E−16 | 2.86E−12 |
| rs2519093 | ABO | 9 | A | 2907 | 1.68 | 8.08E−16 | 3.31E−12 |
| rs505922 | ABO | 9 | G | 2926 | 1.49 | 1.52E−12 | 4.68E−09 |
| rs687289 | ABO | 9 | A | 2924 | 1.48 | 3.03E−12 | 7.46E−09 |
| rs8176719 | ABO | 9 | G | 2900 | 1.47 | 5.68E−12 | 1.16E−08 |
| rs643434 | ABO | 9 | A | 2923 | 1.44 | 3.39E−11 | 5.96E−08 |
| rs630014 | ABO | 9 | A | 2927 | 0.75 | 2.67E−07 | 0.00041 |
| rs3087505^{†} | KLKB1 | 4 | A | 2927 | 0.63 | 4.34E−07 | 0.000593 |
| rs660340 | ABO | 9 | A | 2927 | 0.77 | 1.13E−06 | 0.001389 |
| rs659104 | ABO | 9 | A | 2927 | 0.77 | 1.28E−06 | 0.001425 |
| rs1799963 | F2 | 11 | A | 2891 | 2.46 | 1.69E−06 | 0.001732 |
| rs3917862 | SELP | 1 | G | 2924 | 1.60 | 6.13E−06 | 0.005562 |
| rs4253399^{‡} | F11 | 4 | C | 2927 | 1.28 | 6.33E−06 | 0.005562 |
| rs4525 | F5 | 1 | G | 2924 | 0.77 | 2.34E−05 | 0.01917 |
| rs4524 | F5 | 1 | G | 2927 | 0.77 | 2.51E−05 | 0.01932 |
| rs10158595 | F5 | 1 | A | 2927 | 0.76 | 3.03E−05 | 0.02191 |
| rs6032 | F5 | 1 | G | 2916 | 0.77 | 3.35E−05 | 0.02253 |
| rs5759224 | SCUBE1 | 22 | G | 2864 | 1.43 | 3.48E−05 | 0.02253 |
| rs2213867 | F5 | 1 | G | 2926 | 0.78 | 4.37E−05 | 0.02685 |

*Additive model; adjusted for age, gender, stroke/myocardial infarction and state of residence.
^{†}SNP within 10 kb of F11
^{‡}SNP within 10 kb of KLKB1

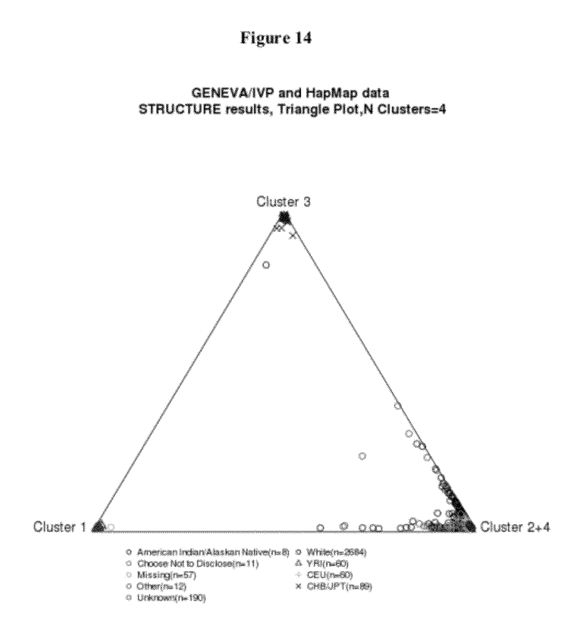

Four additional analyses stratifying by sex, Factor V Leiden (positive/negative for carrier of F5 rs6025 A allele), ABO blood type-O (yes/no based on homozygous deletion in ABO rs8176719 where the homozygous deletion results in ABO blood type-O) and prothrombin G20210A (positive/negative for carrier of F2 rs1799963 A allele) were performed. Similar F5 and ABO SNPs were associated with VTE for both females and males, while F2 rs1799963 was no longer significant for either gender; SNPs within an additional gene (LY86) was significantly associated with VTE among females (Table 5). The odds of VTE appeared to be higher for Factor V Leiden among males. Among Factor V Leiden non carriers, similar ABO and F2 SNPs, and an additional KLKB1 SNP were associated with VTE (Table 6). Among Factor V Leiden carriers, SNPs within PRKCB1 and CD44 were marginally associated with VTE, possibly due to small sample size. Among persons with ABO blood type non-O (ABO rs8176719 G), SNPs within F5, F11 and KLKB1, and an additional gene (GFRA1) were associated with VTE (Table 7). Of note, the ABO rs2519093 remained significantly associated with VTE in this patient subset. Only the Factor V Leiden mutation was significantly associated with VTE among those with the ABO blood type-O (ABO rs8176719 G homozygous deletion). Among prothrombin G20210A non-carriers, SNPs within ABO, F5, F11, KLKB1, SCUBE1 and SELP were significantly associated with VTE (Table 5). The sample size of prothrombin G20210A carriers was insufficient for meaningful analysis. Sex-chromosome analysis was also performed, and no significant results were identified. Q-Q plots of the −log₁₀ p-values for SNP associated with VTE under different analyses showed no evidence of over dispersion in the samples (λ=1.0; FIG. 13).

TABLE 5
Association* of Candidate Gene SNPs with
Venous Thromboembolism Stratified by Sex.

| | | | | | | | |
|---|---|---|---|---|---|---|---|
| | | | Minor | | Odds | | |
| SNP | Gene | Chromosome | Allele | N | Ratio | P-value | Q-value |

Females

| | | | | | | | |
|---|---|---|---|---|---|---|---|
| rs6025 | F5 | 1 | A | 1505 | 2.80 | 8.38E−10 | 1.03E−05 |
| rs6687813 | F5 | 1 | A | 1504 | 2.04 | 4.71E−08 | 0.000289 |
| rs8176719 | ABO | 9 | G | 1487 | 1.51 | 1.38E−07 | 0.000332 |
| rs643434 | ABO | 9 | A | 1503 | 1.51 | 1.45E−07 | 0.000332 |
| rs687289 | ABO | 9 | A | 1503 | 1.51 | 1.52E−07 | 0.000332 |
| rs505922 | ABO | 9 | G | 1505 | 1.51 | 1.62E−07 | 0.000332 |
| rs2519093 | ABO | 9 | A | 1496 | 1.58 | 2.21E−07 | 0.000388 |
| rs1073897 | LY86 | 6 | A | 1496 | 0.68 | 6.41E−06 | 0.009845 |
| rs630014 | ABO | 9 | A | 1505 | 0.71 | 1.26E−05 | 0.01728 |
| rs9328375 | LY86 | 6 | A | 1504 | 0.71 | 2.97E−05 | 0.03656 |

Males

| | | | | | | | |
|---|---|---|---|---|---|---|---|
| rs6025 | F5 | 1 | A | 1422 | 4.28 | 7.37E−14 | 9.06E−10 |
| rs2519093 | ABO | 9 | A | 1411 | 1.82 | 3.46E−10 | 2.13E−06 |
| rs6687813 | F5 | 1 | A | 1422 | 2.20 | 3.98E−09 | 1.63E−05 |
| rs505922 | ABO | 9 | G | 1421 | 1.48 | 1.26E−06 | 0.003881 |
| rs687289 | ABO | 9 | A | 1421 | 1.46 | 2.55E−06 | 0.006264 |
| rs8176719 | ABO | 9 | G | 1413 | 1.44 | 5.11E−06 | 0.01046 |
| rs643434 | ABO | 9 | G | 1421 | 1.39 | 2.7E−05 | 0.04738 |

*Additive model; adjusted for age, stroke/myocardial infarction and state of residence.

TABLE 6
Association* of Candidate Gene SNPs with Venous
Thromboembolism Stratified by Factor V Leiden.

| | | | | | | | |
|---|---|---|---|---|---|---|---|
| | | | Minor | | Odds | | |
| SNP | Gene | Chromosome | Allele | N | Ratio | P-value | Q-value |

Factor V Leiden non-carrier (F5 rs6025 G)

| | | | | | | | |
|---|---|---|---|---|---|---|---|
| rs2519093 | ABO | 9 | A | 2543 | 1.67 | 1.06E−13 | 1.3E−09 |
| rs505922 | ABO | 9 | G | 2557 | 1.46 | 2.33E−10 | 1.43E−06 |
| rs687289 | ABO | 9 | A | 2555 | 1.45 | 5.27E−10 | 2.16E−06 |
| rs8176719 | ABO | 9 | G | 2535 | 1.44 | 7.71E−10 | 2.37E−06 |
| rs643434 | ABO | 9 | A | 2555 | 1.41 | 6.61E−09 | 1.62E−05 |
| rs630014 | ABO | 9 | A | 2558 | 0.74 | 3.76E−07 | 0.00077 |
| rs3087505^{†} | KLKB1 | 4 | A | 2558 | 0.61 | 7.35E−07 | 0.00129 |
| rs1799963 | F2 | 11 | A | 2528 | 2.63 | 9.5E−07 | 0.00146 |

Factor V Leiden carrier (F5 rs6025 A)

| | | | | | | | |
|---|---|---|---|---|---|---|---|
| rs195999 | PRKCB1 | 16 | C | 368 | 0.49 | 2.87E−05 | 0.1778 |
| rs7404320 | PRKCB1 | 16 | G | 369 | 0.49 | 2.89E−05 | 0.1778 |
| rs11033021 | CD44 | 11 | A | 365 | 2.27 | 7.42E−05 | 0.2176 |
| rs196002 | PRKCB1 | 16 | A | 369 | 0.51 | 7.44E−05 | 0.2176 |
| rs8044722 | PRKCB1 | 16 | A | 369 | 0.50 | 8.85E−05 | 0.2176 |
| rs7945310 | CD44 | 11 | A | 369 | 0.46 | 0.000127 | 0.2287 |
| rs9924860 | PRKCB1 | 16 | C | 369 | 1.98 | 0.00013 | 0.2287 |
| rs2188355 | PRKCB1 | 16 | A | 368 | 0.52 | 0.000188 | 0.2887 |
| rs12931116 | PRKCB1 | 16 | G | 368 | 1.92 | 0.000246 | 0.3072 |
| rs11130068 | ITPR1 | 3 | G | 369 | 0.44 | 0.00025 | 0.3072 |

*Additive model; adjusted for age, gender, stroke/myocardial infarction and state of residence.
^{†}SNP is on KLKB1 gene but within 10 kb of F11

TABLE 7
Association* of Candidate Gene SNPs with Venous Thromboembolism
Stratified by ABO non-O Blood Type (ABO rs8170719 [G]).

| | | | | | | | |
|---|---|---|---|---|---|---|---|
| | | | Minor | | Odds | | |
| SNP | Gene | Chromosome | Allele | N | Ratio | P-value | Q-value |

ABO blood type non-O (ABO rs8170719 G)

| | | | | | | | |
|---|---|---|---|---|---|---|---|
| rs6025 | F5 | 1 | A | 1965 | 3.79 | 1.46E−16 | 1.79E−12 |
| rs6687813 | F5 | 1 | A | 1965 | 2.47 | 3.76E−14 | 2.31E−10 |
| rs2519093 | ABO | 9 | A | 1945 | 1.43 | 4.2E−06 | 0.01723 |
| rs3087505^{†} | KLKB1 | 4 | A | 1965 | 0.59 | 7.59E−06 | 0.01873 |
| rs1799963 | F2 | 11 | A | 1933 | 3.09 | 7.62E−06 | 0.01873 |
| rs10749203 | GFRA1 | 10 | A | 1959 | 0.72 | 1.08E−05 | 0.02101 |
| rs8176704 | ABO | 9 | A | 1965 | 0.62 | 1.2E−05 | 0.02101 |
| rs4253399^{‡} | F11 | 4 | C | 1965 | 1.34 | 1.83E−05 | 0.02816 |
| rs881726 | GFRA1 | 10 | G | 1965 | 0.72 | 3.04E−05 | 0.04157 |
| rs10787637 | GFRA1 | 10 | G | 1964 | 0.74 | 3.86E−05 | 0.04743 |

ABO blood type-O (ABO rs8170719 G homozygous deletion)

| | | | | | | | |
|---|---|---|---|---|---|---|---|
| rs6025 | F5 | 1 | A | 962 | 2.72 | 2.18E−06 | 0.02675 |

*Additive model; adjusted for age, gender, stroke/myocardial infarction and state of residence.
^{†}SNP is on KLKB1 gene but within 10 kb of F11
^{‡}SNP is on F11 gene but within 10 kb of KLKB1

TABLE 8
Association* of Candidate Gene SNPs with Venous Thromboembolism
among Prothrombin G20210A (F2 rs1799963 A) Non-Carriers.

| | | | Minor | | Odds | | |
|---|---|---|---|---|---|---|---|
| SNP | Gene | Chromosome | Allele | N | Ratio | P-value | Q-value |
| rs6025 | F5 | 1 | A | 2787 | 3.49 | 2.91E−22 | 3.58E−18 |
| rs6687813 | F5 | 1 | A | 2786 | 2.20 | 1.46E−16 | 8.98E−13 |
| rs2519093 | ABO | 9 | A | 2767 | 1.65 | 3.24E−14 | 1.33E−10 |
| rs505922 | ABO | 9 | G | 2786 | 1.48 | 9.06E−12 | 2.79E−08 |
| rs687289 | ABO | 9 | A | 2786 | 1.47 | 1.39E−11 | 3.42E−08 |
| rs8176719 | ABO | 9 | G | 2760 | 1.46 | 2.39E−11 | 4.9E−08 |
| rs643434 | ABO | 9 | A | 2784 | 1.44 | 9.85E−11 | 1.73E−07 |
| rs630014 | ABO | 9 | A | 2787 | 0.75 | 3.41E−07 | 0.000524 |
| rs3087505^{†} | KLKB1 | 4 | A | 2787 | 0.62 | 5.75E−07 | 0.000786 |
| rs660340 | ABO | 9 | A | 2787 | 0.77 | 2.68E−06 | 0.0033 |
| rs659104 | ABO | 9 | A | 2787 | 0.77 | 3.03E−06 | 0.003388 |
| rs4253399^{‡} | F11 | 4 | C | 2787 | 1.28 | 9.03E−06 | 0.009256 |
| rs3917862 | SELP | 1 | G | 2784 | 1.60 | 1.28E−05 | 0.01214 |
| rs5759224 | SCUBE1 | 22 | G | 2730 | 1.46 | 1.95E−05 | 0.01713 |
| rs4525 | F5 | 1 | G | 2784 | 0.76 | 2.13E−05 | 0.01743 |
| rs4524 | F5 | 1 | G | 2787 | 0.76 | 2.3E−05 | 0.01751 |
| rs10158595 | F5 | 1 | A | 2787 | 0.76 | 2.42E−05 | 0.01751 |
| rs6032 | F5 | 1 | G | 2777 | 0.76 | 2.86E−05 | 0.01951 |
| rs2213867 | F5 | 1 | G | 2784 | 0.77 | 4.02E−05 | 0.02604 |

*Additive model; adjusted for age, gender, stroke/myocardial infarction and state of residence.
^{†}SNP is on KLKB1 gene but within 10 kb of F11
^{‡}SNP is on F11 gene but within 10 kb of KLKB1

The individual, joint, and group population attributable risk (PAR) were calculated for the risk genotypes for Factor V Leiden (F5 rs6025), prothrombin G20210A (F2 rs1799963), ABO blood type non-O (ABO rs8176719), as well as the novel ABO rs2519093 (Table 9). The unadjusted and adjusted individual and joint PARs values were very similar. The highest PAR value was from the ABO blood type non-O, followed by ABO rs2519093, Factor V Leiden, and prothrombin G20210A. The PAR values were very similar between the joint and group estimation methods when either ABO rs8176719 or ABO rs2519093 was included. When both ABO rs8176719 and ABO rs2519093 were included in the group PAR calculation, the method yielded an inflated value of 0.59 compared to the joint method (joint PAR=0.45).

TABLE 9
Attributable Risk (AR) for Significant Single Nucleotide
Polymorphisms (SNPs) Assuming A Dominant Genetic
Model for Each SNP.

| Single Nucleotide | Unadjusted | | Adjusted^{†} | |
|---|---|---|---|---|
| Polymorphism | AR | 95% CI | AR | 95% CI |
| Individual | | | | |
| F5 rs6025 | 0.13 | (0.10, 0.16) | 0.14 | (0.11, 0.16) |
| (Factor V Leiden) | | | | |
| F2 rs1799963 (Prothrombin | 0.04 | (0.02, 0.06) | 0.04 | (0.02, 0.06) |
| G20210A) | | | | |
| ABO rs8176719 (ABO | 0.35 | (0.27, 0.42) | 0.35 | (0.24, 0.47) |
| blood type non-O) | | | | |
| ABO rs2519093 | 0.25 | (0.19, 0.33) | 0.25 | (0.17, 0.32) |
| Joint* | | | | |
| F5 rs6025, F2 rs1799963, | 0.45 | (0.36, 0.54) | 0.45 | (0.37, 0.54) |
| ABO rs8176719 | | | | |
| F5 rs6025, F2 rs1799963, | 0.36 | (0.30, 0.42) | 0.36 | (0.30, 0.42) |
| ABO rs2519093 | | | | |
| F5 rs6025, F2 rs1799963, | 0.45 | (0.36, 0.54) | 0.45 | (0.36, 0.53) |
| ABO rs8176719, | | | | |
| ABO rs2519093 | | | | |
| Group^{‡} | | | | |
| F5 rs6025, F2 rs1799963, | | — | 0.46 | — |
| ABO rs8176719 | | | | |
| F5 rs6025, F2 rs1799963, | | — | 0.39 | — |
| ABO rs2519093 | | | | |
| F5 rs6025, F2 rs1799963, | | — | 0.59 | — |
| ABO rs8176719, | | | | |
| ABO rs2519093 | | | | |

*Bruzzi et al., American Journal of Epidemiology, 122: 904-14 (1985).
^{†}Adjusted for age at blood sample collection, male gender, myocardial infarction/stroke status, and Minnesota residence.
^{‡}Assuming independence between SNPs, i.e., a non-multiplicative interaction between the genotypes.

The results provided herein demonstrate that an association exists between Factor V Leiden, prothrombin G20210A and VTE as well as between ABO blood type non-O and VTE. These results also demonstrate that an ABO intron 1 tag SNP is strongly and independently associated with VTE. Together, these SNPs account for 45% of VTE within this population.

In addition, the results provided herein identified additional SNPs within KLKB1, F11, SELP, SCUBE1, and LY86 that also appear to be strongly associated with VTE. These findings lend further support to the hypothesis that individual genetic variation in genes encoding for components of the procoagulant, anticoagulant, fibrinolytic, and innate immunity pathways predispose to incident VTE.

### Example 3

### A Genome-Wide Association Study of Venous Thromboembolism Identifies Risk Variants in Chromosomes 1q24.2 and 9q

### Genotyping

### Candidate Gene

Leukocyte genomic DNA was extracted, quantified, and diluted to the appropriate concentration for Illumina Infinium iSelect genotyping on all samples collected as described in Example 2. Controls included 2% sample replicates and a CEPH trio for quality control at the Mayo Clinic Technology Center. In addition, case and control DNA sample addresses were randomly assigned across both the 96-well plate as well as the 12-address iSelect BeadChip, insuring approximately equal numbers of case and control DNA samples by each strata to avoid potential plate and chip effects, respectively. Genotyping results from high-quality control DNA (SNP call rate 95%) was used to generate a cluster algorithm. Of the 14,612 SNPs submitted to Illumina from 764 genes within the anticoagulant, procoagulant, fibrinolytic, and innate immunity pathways, 1,585 SNPs (covering 1,100 LD bins) failed manufacture, leaving 13,027 SNPs for analysis.

### GWA

The Center for Inherited Disease Research (CIDR), one of the two genotyping centers supported by the GENEVA consortium (Cornelius et al., Genet. Epidemiol., published online, doi: 10.1002/gepi.20492 (20 Jan. 2010)), genotyped samples using the Illumina Human610-Quad v. 1_B BeadChip (Illumina). The DNA source for all samples came from whole blood. Case and control DNA sample addresses were randomly assigned across 96-well plates provided by CIDR while assuring roughly equal percentages of cases and controls within each plate. Genotype clusters for each SNP were determined using the IlluminaBeadStudio Module (version 3.3.7), and combined intensity data from 99.2% of samples were used to define clusters and call genotypes. Overall, 99.1% of samples attempted (7,114 of 7,178 total) passed quality-control standards. Genotypes were not called if the quality score from BeadStudio was <0.15. Both the mean SNP call rates and the mean sample call rates were 99.8%. Genotypes were released for 589,945 SNPs (99.56% of those attempted). Genotypes were not released for autosomal SNPs with call rates <85%, >1 HapMap replicate error, >1% difference in call rate between genders or >4% difference in heterozygote frequency. Duplicate samples from both HapMap and the study were included on each plate. Reproducibility rates in the raw data were 99.99% among 161 duplicated subjects. Samples included 169 blind duplicates, and 98.8% of these duplicates met quality-control criteria. CIDR's release criteria depended on sample source: samples with >96.5% called genotypes were released for DNA from whole blood.

### Quality-Control Steps

For both CG and GWA, four main categories of quality-control flags were set for autosomal SNPs: (i) unacceptably high rates of missing genotype calls, (ii) low MAF, (iii) unacceptably high rates of Mendelian errors, and (iv) deviation from Hardy-Weinberg equilibrium (HWE).

For the GWA study, 1,965 SNPs with >5% missing genotypes were flagged. A total of 1,683/1,115/1,135 SNPs were flagged overall/cases/controls for HWE deviation at P<10 ⁻⁵. For the CG study, 735 SNPs were excluded due to poor performance (n=554), a MAF<0.005 (n=127), or a call rate <0.95 (n=54), leaving 12,296 SNPs (covering 10,456 bins) for the association analysis.

### Population Stratification

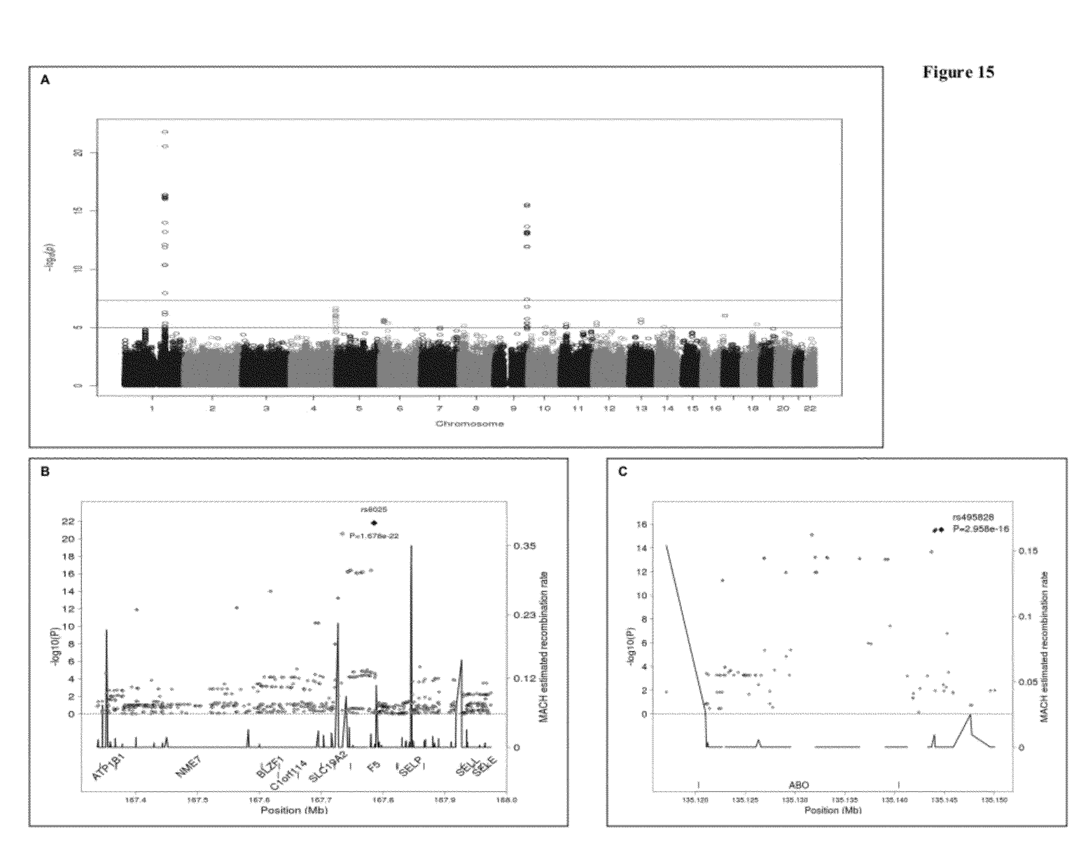

To test for “cryptic relatedness” in the population, a population structure analysis was conducted on all participating subjects to document genetic diversity among the population. Using 494 Ancestry Informative Markers (AIMS) available on both the candidate gene and the GWA studies (Seldin et al., PLoS Genetics, 2:e143 (2006)), STRUCTURE was run on 2962 participants (Pritchard et al., Genetics, 155:945-959 (2000)). The triangle plot provided a graphical representation of genetic structure of the participants plus 209 unrelated individuals from HapMap phase II populations (Yorubans [YRI]; European-Americans from the CEPH collection [CEU]; Chinese from Beijing [CHB]; and Japanese from Tokyo [JPT]), giving a clear sense of how the participants fall among the HapMap reference populations (FIG. 14).

### Imputation

MACH was used for imputation of genotypes to the HapMap phase II CEU reference set of approximately 2.5 million SNPs. All genetic coordinates in tables and figures for this example refer to HapMap release 22 build 36. In regions where no candidate gene genotypes were available, only subjects with GWA data were used for the imputation.

### Replication Samples

Seven SNPs chosen for replication were genotyped in a total of 2,883 individuals (VTE cases, n=1451; controls, n=1432). Replication leukocyte genomic DNA samples were largely from Olmsted County, Minn., USA residents with objectively-diagnosed incident VTE over the 45-year period, and previously-matched Olmsted County resident controls as described elsewhere (Heit et al., Arch. Intern. Med., 160:809-815 (2000); Heit et al., J. Thromb. Haemost., 3:710-17 (2005); and Heit et al., Arterioscler. Thromb. Vasc. Biol., 29:1399-1405 (2009)). The replication case and control sample size was augmented by leukocyte DNA from non-Olmsted County Mayo Clinic patients identified as described for the CG study population for Example 2.

### DNA Sequencing

Sixteen genes were selected for deep sequencing in 84 VTE cases and 12 controls, including 5 genes harboring SNPs significantly associated with VTE (F5, SLC19A2, ABO, NME7, and ATP1B1), 10 genes with SNPs marginally associated with VTE (Clorf114, KLKB1, SELP F11, SCUBE1, PRKCB1, CD44, ITPR1, GFRA1, and BLZF1), and CYP4V2 which reportedly confounds F11 and KLKB1. Agilent SureSelect probes were designed to capture and enrich the Mb genomic regions of these 16 genes. Samples were multiplexed (12-plex) and sequenced using Illumina HiSeq 2000. The sequence reads were aligned to the human genome build 36 using Burrows-Wheeler Aligner, and the single nucleotide variants (SNVs) and small INDELs were called using SNVMix and GATK, respectively.

Capture of the target genomic regions was performed using the Agilent custom eArray. The capturing probes (baits) of 120 bases in length were designed based on the paired-end sequencing protocol with a tiling frequency of 3×. The standard repeat masked regions were avoided based on the definition in the UCSC genome database. The repeat regions were mostly in intronic regions of the genes.

Sequencing was performed using Illumina's HiSeq 2000 sequencer. Twelve samples were multiplexed in each lane of the 8-lane flow cell, and a total of 96 samples (84 VTE cases and 12 controls) were sequenced. The read qualities were examined by FastQC (http://www.bioinformatics.bbsrc.ac.uk/projects/fastqc), which generates QC matrix from the FASTQ files including per-base sequence qualities, per-sequence quality scores, per-base nucleotide content, and sequence duplication levels. The FastQC tool also provided warnings for parameters failing to pass QC thresholds. The paired-end 100-base reads were aligned to human genome build 36 using BWA (Li et al., Annu. Rev. Genomics Hum. Genet., 10:387-406 (2009)), allowing 4% of mismatches with a seed length of 32. If the sequence duplication levels failed to pass the FastQC threshold, the duplicated reads were removed using the SAMtools's rmdup method (Li et al., Annu. Rev. Genomics Hum. Genet., 10:387-406 (2009)). The BWA alignment was then cleaned up and improved using the Genome Analysis ToolKit (GATK) local re-alignments. SNVs were called using SNVMix with a cut-off probability score of 0.8 based on preliminary testing using a HapMap CEPH subject sequenced by the 1000 genome project (data not shown), and INDELs were called by GATK with default parameters setting.

The read depths of each of the A, C, G, T bases at each variant position, as well as the average mapping quality score were provided by curating the BAM pile-up files using SAMtools (Li et al., Annu. Rev. Genomics Hum. Genet., 10:387-406 (2009)). If an identified SNV was a known variant from dbSNP or 1000 Genome Project, the allele frequencies of CEU, YRI, and CHB/JPT populations from HapMap and 1000 Genome Project were provided. Both SNVs and INDELs were annotated by batch submission to the SeattleSeq server, and for SNVs, additional annotations were acquired using a locally or cloud installed SIFT. The SNVs or INDELs within a user defined distance (default: 5 bases) to exon-intron boundaries were flagged as potential splice variants and the corresponding transcript IDs were provided.

Information was reported on genes hosting SNVs and INDELs, including (i) the KEGG pathway(s) (http://www.genome.jp/kegg) to which the gene belongs; and (ii) tissue expression specificity of the gene.

Table 17 provides the start and end base pair locations for the regions sequenced on chromosomes 1 and 9, respectively.

TABLE 17
Selection of genes for the sequencing analysis.

| | Chr | Start | End | Chr | Start | End | Size |
|---|---|---|---|---|---|---|---|
| ATP1B1 | 1 | 169,075,947 | 169,101,960 | 1 | 169,065,947 | 169,609,377 | 543,430 |
| NME7 | 1 | 169,101,771 | 169,337,186 | | | | |
| BLZF1 | 1 | 169,337,194 | 169,365,778 | | | | |
| C1orf114 | 1 | 169,364,114 | 169,396,670 | | | | |
| SLC19A2 | 1 | 169,433,151 | 169,455,208 | | | | |
| F5 | 1 | 169,481,192 | 169,555,769 | | | | |
| SELP | 1 | 169,558,090 | 169,599,377 | | | | |
| ABO | 9 | 136,130,563 | 136,150,630 | 9 | 136,120,563 | 136,160,630 | 40,067 |
| | | | | | | | 583,497 |

### Statistical Methods

Genome-wide scan (Illumina 660Q; 557,112 SNPs) and candidate gene (n=764 genes relevant to the anticoagulant, procoagulant, fibrinolytic, and innate immunity pathways; n=12,551 SNPs) genotypes from Caucasian VTE cases (objectively-diagnosed; no cancer, catheter or antiphospholipid antibodies; n=1270) and controls (frequency-matched on case age, gender, race, MI/stroke status) were merged and imputed to 2.5 million SNPs with MACH using HapMap Phase 2 (60 CEU; Li et al., Annu. Rev. Genomics Hum. Genet., 10:387-406 (2009)). The primary outcome was VTE status, a binary measure. The covariates were age at interview or blood sample collection, sex, state of residence, and stroke/MI status (Table 10). Association between each SNP and VTE was tested for using unconditional logistic regression, adjusting for age, sex, state of residence, and stroke/MI status using PLINK v 1.07 (Purcell et al., Am. J. Human Genet., 81:559-575 (2007)). Similar analysis was used for the replication analysis, and the covariates were age at interview or blood sample collection, sex and state of residence (Table 11). Novel ABO SNVs were tested for an association with VTE using age-, sex-adjusted logistic regression and Fisher's Exact Test.

TABLE 10
Demographic and clinical characteristics by case-control status
for samples in the discovery data set.

| | Case | Control | |
|---|---|---|---|
| Characteristic | n = 1503 | n = 1459 | P-value |
| Patient age, mean ± SD, years | 54.9 ± 16.2 | 55.6 ± 15.7 | 0.225 |
| Male, n (%) | 745 (49.6) | 690 (47.3) | 0.216 |
| Stroke or myocardial infarction, | 285 (19.0) | 150 (10.3) | <0.0001 |
| n (%) | | | |
| U.S. state of residence, n (%) | | | <0.0001 |
| Minnesota | 630 (41.9) | 811 (55.6) | |
| Other states | 873 (58.1) | 648 (44.4) | |

TABLE 11
Significant SNPs with P-values < 10E−05 - adjusted for age, sex, stroke/MI, and U.S. state of residence.

| | | | | | | OR | | | | | Used for |
|---|---|---|---|---|---|---|---|---|---|---|---|
| SNP | CHR | Base Pair | MINOR | GENE | MAF | (95% CI) | P | RSQ | IVP | GWAS | imputation |
| rs6025 | 1 | 167785673 | T | F5 | 0.063 | 3.565 | 1.68E−22 | 0.97 | TRUE | FALSE | TRUE |
| | | | | | | (2.762, 4.602) | | | | | |
| rs1894692 | 1 | 167734278 | G | SLC19A2 | 0.057 | 3.962 | 2.85E−21 | 0.873 | FALSE | FALSE | FALSE |
| | | | | | | (2.98, 5.268) | | | | | |
| rs1018827 | 1 | 167780630 | A | F5 | 0.106 | 2.189 | 4.24E−17 | 0.981 | FALSE | FALSE | FALSE |
| | | | | | | (1.824, 2.628) | | | | | |
| rs6427196 | 1 | 167747847 | C | F5 | 0.106 | 2.174 | 4.24E−17 | 0.998 | FALSE | FALSE | FALSE |
| | | | | | | (1.814, 2.606) | | | | | |
| rs6427195 | 1 | 167747800 | A | F5 | 0.106 | 2.174 | 4.28E−17 | 0.998 | FALSE | FALSE | FALSE |
| | | | | | | (1.814, 2.606) | | | | | |
| rs6427194 | 1 | 167747745 | T | F5 | 0.106 | 2.174 | 4.30E−17 | 0.998 | FALSE | FALSE | FALSE |
| | | | | | | (1.814, 2.606) | | | | | |
| rs970740 | 1 | 167746598 | C | F5 | 0.106 | 2.174 | 4.35E−17 | 0.997 | FALSE | FALSE | FALSE |
| | | | | | | (1.814, 2.606) | | | | | |
| rs6687813 | 1 | 167744198 | A | F5 | 0.104 | 2.172 | 6.24E−17 | 0.997 | TRUE | FALSE | TRUE |
| | | | | | | (1.811, 2.606) | | | | | |
| rs10737547 | 1 | 167742676 | A | F5 | 0.104 | 2.173 | 6.25E−17 | 0.996 | FALSE | FALSE | FALSE |
| | | | | | | (1.812, 2.606) | | | | | |
| rs6427197 | 1 | 167767214 | C | F5 | 0.106 | 2.162 | 6.92E−17 | 0.996 | FALSE | FALSE | FALSE |
| | | | | | | (1.804, 2.591) | | | | | |
| rs6009 | 1 | 167765458 | T | F5 | 0.106 | 2.16 | 7.26E−17 | 0.997 | FALSE | FALSE | FALSE |
| | | | | | | (1.802, 2.588) | | | | | |
| rs2420372 | 1 | 167764680 | A | F5 | 0.106 | 2.159 | 7.47E−17 | 0.997 | FALSE | FALSE | FALSE |
| | | | | | | (1.802, 2.587) | | | | | |
| rs2420370 | 1 | 167757016 | G | F5 | 0.106 | 2.156 | 8.47E−17 | 0.998 | TRUE | FALSE | FALSE |
| | | | | | | (1.799, 2.583) | | | | | |
| rs2420371 | 1 | 167758179 | G | F5 | 0.106 | 2.151 | 9.61E−17 | 0.999 | FALSE | TRUE | TRUE |
| | | | | | | (1.796, 2.578) | | | | | |
| rs649129 | 9 | 135144125 | T | ABO | 0.269 | 1.66 | 2.94E−16 | 0.984 | FALSE | FALSE | FALSE |
| | | | | | | (1.47, 1.874) | | | | | |
| rs495828 | 9 | 135144688 | T | ABO | 0.272 | 1.649 | 2.96E−16 | 0.988 | FALSE | TRUE | TRUE |
| | | | | | | (1.462, 1.859) | | | | | |
| rs579459 | 9 | 135143989 | C | ABO | 0.267 | 1.662 | 3.85E−16 | 0.99 | FALSE | FALSE | FALSE |
| | | | | | | (1.471, 1.878) | | | | | |
| rs2519093 | 9 | 135131691 | A | ABO | 0.243 | 1.685 | 8.08E−16 | NA | TRUE | FALSE | TRUE |
| | | | | | | (1.484, 1.913) | | | | | |
| rs7538157 | 1 | 167618168 | C | BLZF1 | 0.062 | 2.685 | 1.04E−14 | 0.901 | FALSE | FALSE | FALSE |
| | | | | | | (2.091, 3.449) | | | | | |
| rs651007 | 9 | 135143696 | T | ABO | 0.256 | 1.635 | 2.20E−14 | 0.954 | FALSE | FALSE | FALSE |
| | | | | | | (1.441, 1.855) | | | | | |
| rs514659 | 9 | 135132024 | C | ABO | 0.405 | 1.525 | 6.26E−14 | 0.999 | FALSE | FALSE | FALSE |
| | | | | | | (1.366, 1.702) | | | | | |
| rs6696217 | 1 | 167727350 | A | SLC19A2 | 0.133 | 1.965 | 6.33E−14 | 0.822 | FALSE | FALSE | FALSE |
| | | | | | | (1.647, 2.345) | | | | | |
| rs545971 | 9 | 135133193 | T | ABO | 0.404 | 1.525 | 6.38E−14 | 0.998 | FALSE | FALSE | FALSE |
| | | | | | | (1.366, 1.703) | | | | | |
| rs612169 | 9 | 135133263 | G | ABO | 0.404 | 1.524 | 7.21E−14 | 0.997 | FALSE | FALSE | FALSE |
| | | | | | | (1.365, 1.701) | | | | | |
| rs687289 | 9 | 135126927 | A | ABO | 0.404 | 1.524 | 7.42E−14 | 0.999 | TRUE | FALSE | TRUE |
| | | | | | | (1.365, 1.701) | | | | | |
| rs687621 | 9 | 135126886 | G | ABO | 0.404 | 1.523 | 7.58E−14 | 0.998 | FALSE | FALSE | FALSE |
| | | | | | | (1.364, 1.701) | | | | | |
| rs674302 | 9 | 135136485 | A | ABO | 0.404 | 1.522 | 8.14E−14 | 0.998 | FALSE | FALSE | FALSE |
| | | | | | | (1.363, 1.699) | | | | | |
| rs505922 | 9 | 135139050 | C | ABO | 0.403 | 1.52 | 9.39E−14 | 0.999 | TRUE | TRUE | TRUE |
| | | | | | | (1.361, 1.697) | | | | | |
| rs529565 | 9 | 135139321 | C | ABO | 0.403 | 1.52 | 9.45E−14 | 0.999 | FALSE | FALSE | FALSE |
| | | | | | | (1.361, 1.697) | | | | | |
| rs1208327 | 1 | 167563488 | C | NME7 | 0.099 | 2.04 | 7.96E−13 | 0.925 | FALSE | FALSE | FALSE |
| | | | | | | (1.679, 2.48) | | | | | |
| rs643434 | 9 | 135132176 | A | ABO | 0.423 | 1.484 | 1.16E−12 | 0.999 | TRUE | FALSE | TRUE |
| | | | | | | (1.331, 1.654) | | | | | |
| rs644234 | 9 | 135132038 | G | ABO | 0.423 | 1.484 | 1.16E−12 | 0.999 | FALSE | FALSE | FALSE |
| | | | | | | (1.331, 1.654) | | | | | |
| rs657152 | 9 | 135129086 | A | ABO | 0.423 | 1.483 | 1.21E−12 | 0.999 | FALSE | TRUE | TRUE |
| | | | | | | (1.33, 1.653) | | | | | |
| rs16861990 | 1 | 167401751 | C | NME7 | 0.099 | 2.019 | 1.34E−12 | 0.932 | FALSE | TRUE | TRUE |
| | | | | | | (1.662, 2.451) | | | | | |
| rs8176719 | 9 | 135122729 | G | ABO | 0.419 | 1.469 | 5.68E−12 | NA | TRUE | FALSE | TRUE |
| | | | | | | (1.317, 1.639) | | | | | |
| rs1208135 | 1 | 167690722 | A | SLC19A2 | 0.09 | 1.926 | 4.26E−11 | 0.93 | FALSE | FALSE | FALSE |
| | | | | | | (1.585, 2.34) | | | | | |
| rs1208134 | 1 | 167695568 | C | SLC19A2 | 0.09 | 1.924 | 4.46E−11 | 0.933 | FALSE | TRUE | TRUE |
| | | | | | | (1.584, 2.338) | | | | | |
| rs2038024 | 1 | 167722606 | C | SLC19A2 | 0.177 | 1.533 | 1.12E−08 | 0.893 | FALSE | TRUE | TRUE |
| | | | | | | (1.324, 1.775) | | | | | |
| rs630014 | 9 | 135139543 | A | ABO | 0.42 | 0.736 | 3.85E−08 | 0.999 | TRUE | TRUE | TRUE |
| | | | | | | (0.66, 0.821) | | | | | |
| rs633862 | 9 | 135145265 | C | ABO | 0.404 | 0.75 | 1.68E−07 | 0.982 | FALSE | FALSE | FALSE |
| | | | | | | (0.674, 0.836) | | | | | |
| rs3756008 | 4 | 187422379 | T | F11 | 0.418 | 1.337 | 2.16E−07 | 0.952 | FALSE | FALSE | FALSE |
| | | | | | | (1.198, 1.493) | | | | | |
| rs2102575 | 4 | 187368498 | G | CYP4V2 | 0.1 | 0.623 | 3.32E−07 | 0.98 | FALSE | TRUE | TRUE |
| | | | | | | (0.519, 0.747) | | | | | |
| rs3087505 | 4 | 187416480 | A | KLKB1 | 0.1 | 0.628 | 4.22E−07 | 0.998 | TRUE | FALSE | TRUE |
| | | | | | | (0.524, 0.752) | | | | | |
| rs12061601 | 1 | 167337074 | C | ATP1B1 | 0.146 | 1.508 | 5.03E−07 | 0.929 | FALSE | TRUE | TRUE |
| | | | | | | (1.285, 1.77) | | | | | |
| rs2901092 | 1 | 167723017 | G | SLC19A2 | 0.072 | 1.872 | 7.81E−07 | 0.674 | FALSE | FALSE | FALSE |
| | | | | | | (1.459, 2.4) | | | | | |
| rs925451 | 4 | 187424563 | A | F11 | 0.413 | 1.311 | 8.65E−07 | 0.994 | FALSE | TRUE | TRUE |
| | | | | | | (1.177, 1.461) | | | | | |
| rs3756009 | 4 | 187423105 | G | F11 | 0.413 | 1.312 | 8.78E−07 | 0.988 | FALSE | FALSE | FALSE |
| | | | | | | (1.177, 1.462) | | | | | |
| rs11655838 | 17 | 5663930 | A | NLRP1 | 0.481 | 1.328 | 9.68E−07 | 0.851 | FALSE | TRUE | TRUE |
| | | | | | | (1.185, 1.487) | | | | | |
| rs1877320 | 4 | 187368273 | G | CYP4V2 | 0.104 | 0.641 | 1.02E−06 | 0.974 | FALSE | FALSE | FALSE |
| | | | | | | (0.536, 0.766) | | | | | |
| rs9995366 | 4 | 187368378 | T | CYP4V2 | 0.104 | 0.642 | 1.12E−06 | 0.976 | FALSE | TRUE | TRUE |
| | | | | | | (0.537, 0.767) | | | | | |
| rs660340 | 9 | 135137374 | A | ABO | 0.408 | 0.766 | 1.13E−06 | NA | TRUE | FALSE | TRUE |
| | | | | | | (0.689, 0.853) | | | | | |
| rs6842047 | 4 | 187370570 | A | CYP4V2 | 0.104 | 0.642 | 1.16E−06 | 0.975 | FALSE | FALSE | FALSE |
| | | | | | | (0.537, 0.768) | | | | | |
| rs659104 | 9 | 135137644 | A | ABO | 0.408 | 0.767 | 1.28E−06 | NA | TRUE | FALSE | TRUE |
| | | | | | | (0.689, 0.854) | | | | | |
| rs1799963 | 11 | 46717631 | A | F2 | 0.025 | 2.455 | 1.69E−06 | NA | TRUE | FALSE | TRUE |
| | | | | | | (1.7, 3.546) | | | | | |
| rs4253399 | 4 | 187425088 | G | F11 | 0.411 | 1.301 | 1.69E−06 | 0.996 | TRUE | TRUE | TRUE |
| | | | | | | (1.168, 1.449) | | | | | |
| rs3758348 | 9 | 135229220 | C | SURF4 | 0.169 | 1.477 | 1.79E−06 | 0.765 | FALSE | FALSE | FALSE |
| | | | | | | (1.258, 1.733) | | | | | |
| rs1323922 | 13 | 66385414 | G | PCDH9 | 0.12 | 0.654 | 2.13E−06 | 0.842 | FALSE | TRUE | TRUE |
| | | | | | | (0.549, 0.78) | | | | | |
| rs12192563 | 6 | 16201441 | A | MYLIP | 0.494 | 0.763 | 2.28E−06 | 0.86 | FALSE | FALSE | FALSE |
| | | | | | | (0.682, 0.853) | | | | | |
| rs9396643 | 6 | 16200972 | T | MYLIP | 0.494 | 0.763 | 2.28E−06 | 0.861 | FALSE | TRUE | TRUE |
| | | | | | | (0.682, 0.853) | | | | | |
| rs9358084 | 6 | 16200504 | G | MYLIP | 0.494 | 0.763 | 2.33E−06 | 0.86 | FALSE | FALSE | FALSE |
| | | | | | | (0.682, 0.854) | | | | | |
| rs9358083 | 6 | 16200051 | G | MYLIP | 0.494 | 0.763 | 2.34E−06 | 0.859 | FALSE | FALSE | FALSE |
| | | | | | | (0.682, 0.854) | | | | | |
| rs7768105 | 6 | 16199373 | T | MYLIP | 0.493 | 0.762 | 2.39E−06 | 0.853 | FALSE | FALSE | FALSE |
| | | | | | | (0.681, 0.853) | | | | | |
| rs4716045 | 6 | 16204575 | T | MYLIP | 0.489 | 1.312 | 2.49E−06 | 0.861 | FALSE | FALSE | FALSE |
| | | | | | | (1.172, 1.469) | | | | | |
| rs12201671 | 6 | 16205941 | C | MYLIP | 0.488 | 1.311 | 2.60E−06 | 0.862 | FALSE | FALSE | FALSE |
| | | | | | | (1.171, 1.468) | | | | | |
| rs6459448 | 6 | 16206269 | A | MYLIP | 0.488 | 1.311 | 2.66E−06 | 0.862 | FALSE | TRUE | TRUE |
| | | | | | | (1.171, 1.467) | | | | | |
| rs9383114 | 6 | 16207259 | A | MYLIP | 0.488 | 1.31 | 2.88E−06 | 0.861 | FALSE | FALSE | FALSE |
| | | | | | | (1.17, 1.467) | | | | | |
| rs2289252 | 4 | 187444375 | T | F11 | 0.415 | 1.316 | 2.89E−06 | 0.859 | FALSE | FALSE | FALSE |
| | | | | | | (1.173, 1.477) | | | | | |
| rs6928267 | 6 | 16208132 | G | MYLIP | 0.488 | 1.309 | 2.94E−06 | 0.861 | FALSE | FALSE | FALSE |
| | | | | | | (1.169, 1.466) | | | | | |
| rs10949343 | 6 | 16209236 | C | MYLIP | 0.494 | 1.31 | 2.94E−06 | 0.852 | FALSE | FALSE | FALSE |
| | | | | | | (1.17, 1.468) | | | | | |
| rs6912988 | 6 | 16208633 | C | MYLIP | 0.488 | 1.309 | 3.00E−06 | 0.861 | FALSE | FALSE | FALSE |
| | | | | | | (1.169, 1.466) | | | | | |
| rs12192575 | 6 | 16210281 | T | MYLIP | 0.487 | 1.307 | 3.34E−06 | 0.862 | FALSE | TRUE | TRUE |
| | | | | | | (1.168, 1.463) | | | | | |
| rs7745965 | 6 | 16210721 | A | MYLIP | 0.487 | 1.307 | 3.47E−06 | 0.86 | FALSE | FALSE | FALSE |
| | | | | | | (1.167, 1.463) | | | | | |
| rs7749877 | 6 | 16210781 | A | MYLIP | 0.487 | 1.307 | 3.70E−06 | 0.856 | FALSE | FALSE | FALSE |
| | | | | | | (1.167, 1.463) | | | | | |
| rs10492593 | 13 | 66392118 | G | PCDH9 | 0.116 | 0.654 | 3.82E−06 | 0.825 | FALSE | FALSE | FALSE |
| | | | | | | (0.546, 0.783) | | | | | |
| rs11832404 | 12 | 13897572 | C | GRIN2B | 0.039 | 0.478 | 3.94E−06 | 0.841 | FALSE | FALSE | FALSE |
| | | | | | | (0.349, 0.654) | | | | | |
| rs11833339 | 12 | 13899994 | G | GRIN2B | 0.039 | 0.478 | 3.95E−06 | 0.841 | FALSE | FALSE | FALSE |
| | | | | | | (0.349, 0.654) | | | | | |
| rs8176681 | 9 | 135129575 | C | ABO | 0.384 | 0.775 | 4.15E−06 | 0.996 | FALSE | FALSE | FALSE |
| | | | | | | (0.695, 0.864) | | | | | |
| rs2073827 | 9 | 135126954 | C | ABO | 0.384 | 0.775 | 4.34E−06 | 0.996 | FALSE | FALSE | FALSE |
| | | | | | | (0.695, 0.864) | | | | | |
| rs3917862 | 1 | 167859737 | G | SELP | 0.075 | 1.61 | 4.35E−06 | 0.999 | TRUE | TRUE | TRUE |
| | | | | | | (1.314, 1.972) | | | | | |
| rs3134929 | 6 | 32300085 | G | NOTCH4 | 0.188 | 0.711 | 4.43E−06 | 0.918 | FALSE | FALSE | FALSE |
| | | | | | | (0.614, 0.822) | | | | | |
| rs4253417 | 4 | 187435999 | C | F11 | 0.427 | 1.306 | 4.88E−06 | 0.86 | FALSE | FALSE | FALSE |
| | | | | | | (1.165, 1.465) | | | | | |
| rs4478991 | 11 | 22155841 | A | TMEM16E | 0.436 | 0.765 | 5.19E−06 | 0.828 | FALSE | FALSE | FALSE |
| | | | | | | (0.681, 0.858) | | | | | |
| rs937798 | 18 | 60582714 | T | SERPINB8 | 0.384 | 0.77 | 5.27E−06 | 0.873 | FALSE | FALSE | FALSE |
| | | | | | | (0.688, 0.862) | | | | | |
| rs11830268 | 12 | 13916583 | A | GRIN2B | 0.041 | 0.498 | 6.35E−06 | 0.826 | FALSE | FALSE | FALSE |
| | | | | | | (0.368, 0.674) | | | | | |
| rs11835020 | 12 | 13907877 | C | GRIN2B | 0.041 | 0.499 | 6.65E−06 | 0.829 | FALSE | TRUE | TRUE |
| | | | | | | (0.369, 0.675) | | | | | |
| rs12546956 | 8 | 18405665 | T | PSD3 | 0.115 | 0.656 | 7.18E−06 | 0.767 | FALSE | FALSE | FALSE |
| | | | | | | (0.545, 0.788) | | | | | |
| rs10919168 | 1 | 167660850 | A | C1orf114 | 0.31 | 1.317 | 7.66E−06 | 0.874 | FALSE | FALSE | FALSE |
| | | | | | | (1.168, 1.486) | | | | | |
| rs3124765 | 9 | 135318478 | T | C9orf7 | 0.144 | 1.658 | 8.44E−06 | 0.454 | FALSE | FALSE | FALSE |
| | | | | | | (1.327, 2.071) | | | | | |
| rs4244490 | 11 | 22135551 | A | TMEM16E | 0.427 | 0.771 | 8.70E−06 | 0.84 | FALSE | FALSE | FALSE |
| | | | | | | (0.687, 0.865) | | | | | |
| rs4275631 | 11 | 22138940 | T | TMEM16E | 0.427 | 0.771 | 8.70E−06 | 0.844 | FALSE | FALSE | FALSE |
| | | | | | | (0.688, 0.865) | | | | | |
| rs4962153 | 9 | 135313575 | A | ADAMTS13 | 0.165 | 1.52 | 9.10E−06 | 0.586 | FALSE | FALSE | FALSE |
| | | | | | | (1.264, 1.829) | | | | | |
| rs9323164 | 14 | 48222321 | T | RPS29 | 0.409 | 0.771 | 9.12E−06 | 0.853 | FALSE | FALSE | FALSE |
| | | | | | | (0.688, 0.865) | | | | | |
| rs739468 | 9 | 135316069 | T | C9orf7 | 0.165 | 1.523 | 9.28E−06 | 0.582 | FALSE | FALSE | FALSE |
| | | | | | | (1.264, 1.833) | | | | | |
| rs12029857 | 1 | 167417852 | C | NME7 | 0.114 | 1.617 | 9.39E−06 | 0.656 | FALSE | FALSE | FALSE |
| | | | | | | (1.307, 2) | | | | | |
| rs1557570 | 1 | 167774468 | T | F5 | 0.325 | 0.772 | 9.51E−06 | 0.939 | FALSE | FALSE | FALSE |
| | | | | | | (0.688, 0.866) | | | | | |
| rs17490626 | 10 | 70888652 | C | TSPAN15 | 0.119 | 0.673 | 9.56E−06 | 0.825 | FALSE | FALSE | FALSE |
| | | | | | | (0.565, 0.802) | | | | | |
| rs7481951 | 11 | 22228446 | A | TMEM16E | 0.42 | 0.773 | 9.59E−06 | 0.852 | FALSE | FALSE | FALSE |
| | | | | | | (0.689, 0.866) | | | | | |
| rs1563455 | 4 | 190530151 | T | FRG1 | 0.248 | 0.714 | 9.98E−06 | 0.671 | FALSE | FALSE | FALSE |
| | | | | | | (0.615, 0.829) | | | | | |

### URLs

PLINK, http://pngu.mgh.harvard.edu/˜purcell/plink/; public sequence primers,
http://genetics.uiowa.edu; Phred, Phrap, Consed,
http://www.phrap.org/phredphrapconsed.html; PolyPhred,
http://droog.gs.washington.edu/polyphred/; PolyPhen,
http://genetics.bwh.harvard.edu/pph/.

### Results

Results from a merged GWAS and candidate gene genotyping study with additional genotypes from imputation using a larger sample size of U.S. VTE cases of non-Hispanic European-ancestry, and U.S. controls frequency-matched on age and gender were obtained (Table 10). From population stratification analysis, 98.64% of samples were classified as European and 1.36% as “other” ancestry (includes individuals of mixed ancestry; FIG. 14).

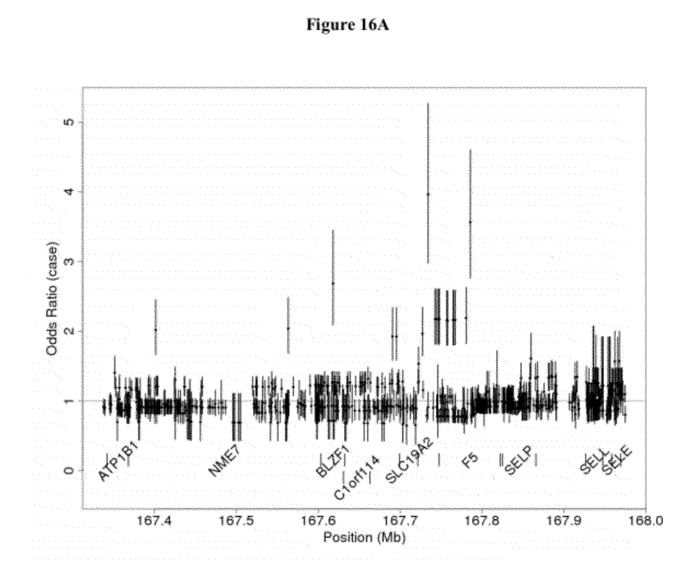

After adjusting for age, gender, U.S. state of residence, and stroke/MI status, 39 SNPs exceeded genome-wide significance of 5×10E-8 (FIG. 15 and Table 11); F2 rs1799963 (Prothrombin G20210A) was borderline significant (OR=2.46, p=1.7E-06). Table 12 shows the association results for significant SNPs not in linkage disequilibrium within a gene (F5 rs6025, BLZF1 rs7538157, NME7 rs16861990, SLC19A2 rs2038024, and ABO rs2519093, rs8176719 [blood group non-O] and rs495828). BLZF1 rs7538157 was in high linkage disequilibrium (LD) with F5 rs6025, SLC19A2 rs2038024, and ATP1B1 rs12061601. In a replication study (Table 13), adjusted for age, gender and U.S. state of residence, F5 rs6025 (p=1.4E-12), NME7 rs16861990 (p=4.9E-9), ATP1B1 rs12061601 (p=0.02), ABO rs2519093 (p=1.2E-17), rs495828 (p=2.4E-17), and rs8176719 (p=5.7E-16), and F2 rs1799963 were significantly associated with VTE, and SLC19A2 rs2038024 (p=0.09) was marginally associated (Table 14). All replicated SNP ORs were in the same direction and of similar magnitude as that of the GWAS.

TABLE 12
Odds ratio and 95% confidence intervals, minor allele, minor allele frequency (MAF)
and imputation quality for single nucleotide polymorphisms (SNPs) showing genome-
wide significance, adjusted for age, gender, stroke/MI and state of residence.

| | | | | | | | | |
|---|---|---|---|---|---|---|---|---|
| | | | Minor | | | | | Imputed (r²)/ |
| SNP | Chr | Position^{†} | allele | Gene | MAF | OR (95% CI) | P-value | Genotype |

SNPs in genes previously reported

| | | | | | | | | |
|---|---|---|---|---|---|---|---|---|
| rs6025 | 1 | 167785673 | T | F5 | 0.063 | 3.57 (2.76, 4.60) | 1.68E−22 | Yes (0.97)/Yes |
| rs8176719 | 9 | 135122729 | G | ABO | 0.419 | 1.47 (1.32, 1.64) | 5.68E−12 | No/Yes^{‡} |
| rs2519093 | 9 | 135131691 | A | ABO | 0.243 | 1.69 (1.48, 1.91) | 8.08E−16 | No/Yes^{‡} |

SNPs in genes not previously reported

| | | | | | | | | |
|---|---|---|---|---|---|---|---|---|
| rs7538157 | 1 | 167618168 | C | BLZF1 | 0.062 | 2.69 (2.09, 3.45) | 1.04E−14 | Yes (0.901)/No |
| rs16861990 | 1 | 167401751 | C | NME7 | 0.099 | 2.02 (1.66, 2.45) | 1.69E−12 | Yes (0.932)/Yes^{§} |
| rs2038024 | 1 | 167722606 | C | SLC19A2 | 0.177 | 1.53 (1.32, 1.78) | 1.12E−08 | Yes (0.893)/Yes^{§} |
| rs495828 | 9 | 135144688 | T | ABO | 0.272 | 1.65 (1.46, 1.86) | 2.96E−16 | Yes (0.988)/Yes^{§} |

^{†}NCBI build 36 of the human genome
^{‡}Genotype available in all subjects through the candidate gene data set.
^{§}Genotype available in only 2570 subjects (87% of our combined samples) through the GWA data set

TABLE 13
Demographic characteristics by case-control status for samples
in the replication data set.

| | Cases | Controls | |
|---|---|---|---|
| Characteristics | n = 1407 | n = 1418 | Total |
| Patient age, mean ± SD, years | 60.8 ± 16.7 | 64.9 ± 12.8 | 62.9 ± 15.0 |
| Male, n (%) | 47.1 | 48.0 | 47.4 |
| Olmsted County, MN | 52.4 | 51.7 | 52.0 |
| resident, n (%) | | | |
| Minnesota residents, n (%) | 67.2 | 70.7 | 69.0 |

TABLE 14
Replication odds ratio and 95% confidence intervals, minor allele, minor allele
frequency (MAF; overall and by case/control status) using 2,825 subjects (1407
cases and 1418 controls), adjusted for age, gender and U.S. state of residence.

| SNP | Gene | N | MAF | MAF - cases | MAF - controls | OR (95% CI) | P value |
|---|---|---|---|---|---|---|---|
| rs6025 | F5 | 2543 | 0.059 | 0.085 | 0.034 | 2.6 (2.0, 3.3) | 1.40E−12 |
| rs16861990 | NME7 | 2559 | 0.095 | 0.122 | 0.069 | 1.8 (1.5, 2.2) | 4.94E−09 |
| rs12061601 | ATP1B1 | 2521 | 0.140 | 0.154 | 0.127 | 1.2 (1.0, 1.4) | 0.02 |
| rs2038024 | SLC19A2 | 2603 | 0.171 | 0.182 | 0.159 | 1.1 (1.0, 1.3) | 0.09 |
| rs2519093 | ABO | 2599 | 0.234 | 0.286 | 0.183 | 1.8 (1.6, 2.1) | 1.17E−17 |
| rs495828 | ABO | 2627 | 0.260 | 0.314 | 0.207 | 1.7 (1.5, 2.0) | 2.41E−17 |
| rs8176719 | ABO | 2532 | 0.415 | 0.472 | 0.357 | 1.6 (1.5, 1.8) | 5.68E−16 |
| rs1799963 | F2 | 2620 | 0.020 | 0.024 | 0.015 | 1.6 (1.1, 2.4) | 0.03 |

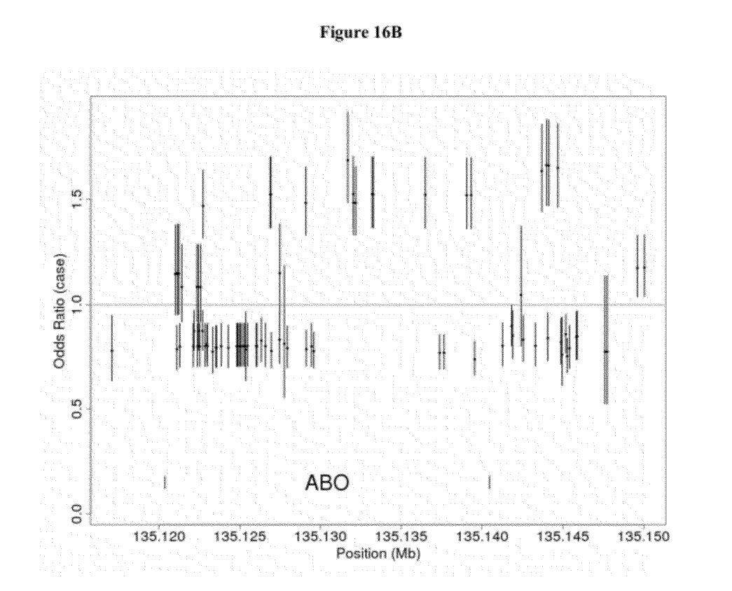

These SNPs cluster in two genomic regions located on chromosome 1q (FIG. 16A) and on chromosome 9q (FIG. 16B). Since NME7, ATP1B1, SLC19A2, BLZF1, SELL, and SELP are in close proximity to F5, the association analysis including F5 rs6025 (Factor V Leiden) as a covariate was repeated, and only the ABO SNPs remained significantly associated with VTE at the genome-wide level. Similarly, in the replication study, only the ABO SNPs remained statistically significant after adjusting for F5 rs6025 (Table 15). Receiver Operating Characteristic (ROC) areas under the curve (AUC), calculated individually and jointly using all significant SNPs, were not appreciably different for covariates plus F5 rs6025 (AUC=0.653) vs. covariates plus F5 rs6025, NME7 rs16861990, BLZF1 rs7538157 vs. ATP1B1 rs12061601 and SLC19A2 rs2038024 (AUC=0.654).

TABLE 15
Replication odds ratio and 95% confidence intervals using
2,825 subjects (1407 cases and 1418 controls), adjusted for
age, gender, U.S. state of residence and F5 rs6025 (Factor V Leiden).

| SNP | Gene | N | OR (95% CI) | P value |
|---|---|---|---|---|
| rs16861990 | NME7 | 2631 | 1.2 (0.9, 1.6) | 0.13 |
| rs12061601 | ATP1B1 | 2605 | 0.9 (0.8, 1.1) | 0.52 |
| rs2038024 | SLC19A2 | 2601 | 0.8 (0.7, 1.0) | 0.01 |
| rs2519093 | ABO | 2601 | 1.8 (1.6, 2.1) | 3.2E−17 |
| rs495828 | ABO | 2629 | 1.7 (1.5, 2.0) | 8.91E−17 |
| rs8176719 | ABO | 2603 | 1.6 (1.4, 1.8) | 1.97E−13 |
| rs1799963 | F2 | 2609 | 1.7 (1.1, 2.5) | 0.02 |

To further evaluate ABO sequence variation for an association with VTE, ABO deep sequencing including 10 Kb of the flanking regions using Illumina HiSeq 2000 was performed. Excluding the intronic repeat regions, 98% of the targeted area was sequenced with >20× coverage in 96 samples (82 VTE cases and 14 controls). On average, ˜600 SNVs and ˜50 INDELs were detected in each sample. Fifteen novel single nucleotide variations (SNVs) in ABO intron 6 and the ABO 3′ UTR were associated with VTE (p<E-06) and belonged to three distinctive LD blocks; none were in LD with ABO rs8176719 or rs2519093 (Table 16). SNVs inside the middle LD block at the 3′ of ABO were located within an enhancer and promoter histone marked with putative transcription factor binding sites. In addition, strong evidence from both ENCODE and dbEST supported the middle LD block as lying within a novel transcript, probably an extension of the 3′ of ABO. In addition, a novel, significant, protective, frame-shifting single base (G) deletion was discovered at ABO chr9:135120877.

TABLE 16
Summary of the significant SNVs.

| | | | | MAF, N | MAF, N | MAF, N | MAF, N | MAF, N | |
|---|---|---|---|---|---|---|---|---|---|
| | | Major | Minor | Controls | Controls | Cases | Cases | Cases | |
| Position | Genomic Location | Allele | Allele | 1/0 | 2/1 | 0/0 | 1/1 | 2/2 | P-value |
| 135111124^{†} | 10 kb 3′ of ABO | C | T | 0.30, 10 | 0.38, 4 | 0.04, 28 | 0.02, 27 | 0.02, 27 | 3.78E−07 |
| 135111447 | 10 kb 3′ of ABO | G | A | 0.30, 10 | 0.38, 4 | 0.02, 28 | 0.00, 27 | 0.02, 27 | 2.43E−08 |
| 135111473 | 10 kb 3′ of ABO | A | G | 0.35, 10 | 0.50, 4 | 0.05, 28 | 0.04, 27 | 0.02, 27 | 1.05E−08 |
| 135112074 | 9 kb 3′ of ABO | T | C | 0.30, 10 | 0.38, 4 | 0.04, 28 | 0.02, 27 | 0.02, 27 | 3.78E−07 |
| 135112527 | 9 kb 3′ of ABO | T | C | 0.35, 10 | 0.50, 4 | 0.05, 28 | 0.04, 27 | 0.02, 27 | 1.05E−08 |
| 135116452 | 4 kb 3′ of ABO | C | A | 0.15, 10 | 0.50, 4 | 0.02, 28 | 0.02, 27 | 0.00, 27 | 2.74E−06 |
| 135117089^{‡} | 4 kb 3′ of ABO | T | C | 0.30, 10 | 0.00, 4 | 0.00, 28 | 0.02, 27 | 0.02, 27 | 4.02E−05 |
| 135117302 | 3 kb 3′ of ABO | A | G | 0.30, 10 | 0.00, 4 | 0.00, 28 | 0.00, 27 | 0.02, 27 | 6.35E−06 |
| 135117426 | 3 kb 3′ of ABO | C | G | 0.28, 9 | 0.00, 4 | 0.00, 28 | 0.00, 27 | 0.00, 26 | 3.80E−06 |
| 135117777 | 3 kb 3′ of ABO | A | C | 0.30, 10 | 0.00, 4 | 0.00, 28 | 0.00, 27 | 0.02, 27 | 6.35E−06 |
| 135121716^{§} | ABO intron 6 | C | T | 0.11, 9 | 0.00, 4 | 0.02, 28 | 0.00, 27 | 0.00, 27 | 4.76E−05 |
| 135121989 | ABO intron 6 | T | C | 0.15, 10 | 0.50, 3 | 0.02, 28 | 0.00, 27 | 0.00, 27 | 6.00E−07 |
| 135122337 | ABO intron 6 | G | A | 0.11, 9 | 0.50, 4 | 0.02, 28 | 0.00, 27 | 0.00, 27 | 4.21E−06 |
| 135122346 | ABO intron 6 | T | C | 0.15, 10 | 0.50, 4 | 0.02, 28 | 0.00, 27 | 0.00, 27 | 6.00E−07 |
| 135122575 | ABO intron 6 | C | A | 0.15, | 0.50 | 0.02 | 0 | 0 | 6.00E−07 |

^{†}LD block (r² = 0.62-0.83)
^{‡}LD block (r² > 0.80)
^{§}LD block (r² > 0.85)
None of the significant SNVs are in high LD with the two SNPs used to select cases and controls (rs8176719 and rs2519093).

These results demonstrate that SNVs within three ABO LD blocks are significantly associated with VTE.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for identifying a mammal having increased susceptibility to develop venous thromboembolism, wherein said method comprises:
(a) determining whether or not a mammal contains a T allele of rs2519093, and
(b) classifying said mammal as having an increased susceptibility to develop venous thromboembolism if said mammal contains said T allele.

2. The method of claim 1, wherein said mammal is a human.

3. The method of claim 1, wherein said method comprises determining whether or not said mammal is heterozygous for said T allele, wherein said mammal has an increased susceptibility to develop venous thromboembolism if said mammal is heterozygous for said T allele.

4. The method of claim 1, wherein said method comprises determining whether or not said mammal is homozygous for said T allele, wherein said mammal has an increased susceptibility to develop venous thromboembolism if said mammal is homozygous for said T allele.

5. The method of claim 1, wherein said method comprises determining whether or not said mammal contains a G allele of rs6025, a G allele of rs1799963, or a mutant allele of rs8176719.

6. A method for identifying a mammal having increased susceptibility to develop venous thromboembolism, wherein said method comprises:
(a) detecting the presence of a mutant T allele of rs2519093 in a mammal, and
(b) classifying said mammal as having an increased susceptibility to develop venous thromboembolism based at least in part on the presence of said mutant T allele.

7. The method of claim 6, wherein said mammal is a human.

8. The method of claim 6, wherein said method comprises determining that said mammal is heterozygous for said mutant T allele.

9. The method of claim 6, wherein said method comprises determining that said mammal is homozygous for said mutant T allele.

10. The method of claim 6, wherein said method comprises detecting the presence of a G allele of rs6025, a G allele of rs1799963, or a mutant allele of rs8176719.

11. A method for identifying a mammal having increased susceptibility to develop venous thromboembolism, wherein said method comprises:
(a) detecting the presence of a mutant T allele of rs2519093 in a mammal,
(b) detecting the presence of a G allele of rs6025, a G allele of rs1799963, or a mutant allele of rs8176719, and
(c) classifying said mammal as having an increased susceptibility to develop venous thromboembolism based at least in part on the presence of said mutant T allele and the presence of said G allele of rs6025, said G allele of rs1799963, or said mutant allele of rs8176719.

12. The method of claim 11, wherein said mammal is a human.

13. The method of claim 11, wherein said method comprises determining that said mammal is heterozygous for said mutant T allele.

14. The method of claim 11, wherein said method comprises determining that said mammal is homozygous for said mutant T allele.

15. A method for identifying a mammal having increased susceptibility to develop venous thromboembolism, wherein said method comprises:
(a) determining whether or not a mammal contains as allele of rs3087505, rs4253399, rs3917862, rs5759224, rs1073897, rs9328375, rs7538157, rs16861990, rs2038024, rs495828, or rs8176719 that is associated with a susceptibility to develop venous thromboembolism, and
(b) classifying said mammal as having an increased susceptibility to develop venous thromboembolism if said mammal contains said allele.

16. A method for identifying a mammal having increased susceptibility to develop venous thromboembolism, wherein said method comprises:
(a) detecting the presence of, in a mammal, an allele of rs3087505, rs4253399, rs3917862, rs5759224, rs1073897, rs9328375, rs7538157, rs16861990, rs2038024, rs495828, or rs8176719 that is associated with a susceptibility to develop venous thromboembolism, and
(b) classifying said mammal as having an increased susceptibility to develop venous thromboembolism based at least in part on the presence of said mutant T allele.
